(19) United States
(12) Patent Application Publication
(10) Pub. No.: US 2009/0093506 A1
(43) Pub. Date: Apr. 9, 2009

(54) Copper antagonist compositions

(76) Inventor: Garth J.S. Cooper, Ponsonby (NZ)

Correspondence Address: DUANE MORRIS LLP - San Diego, 101 WEST BROADWAY, SUITE 900, SAN DIEGO, CA, 92101-8285, US

(21) Appl. No.: 11/973,270

(22) Filed: Oct. 4, 2007

(51) Int. Cl. A61K 31/505; A61K 31/351; A61K 31/40; A61K 31/225; A61P 19/00; A61P 3/00; A61K 31/366; A61K 31/403

(52) U.S. Cl. 514/275; 514/460; 514/419; 514/423; 514/529

(57) ABSTRACT

Pharmaceutical compositions having a pharmaceutically acceptable copper antagonist compound(s) or a pharmaceutically acceptable salt or prodrug thereof, including copper (II) antagonists, and a pharmaceutically acceptable 3-hydroxy-3-methylglutaryl coenzyme A reductase inhibitor or a pharmaceutically acceptable salt or prodrug thereof, articles and kits and delivery devices containing such compositions, tablets and capsules and formulations containing such compositions, and methods of use for treatment of subjects, including humans, who have or are at risk for various diseases, disorders, and conditions.

### FIELD

Compositions containing a pharmaceutically acceptable copper antagonist compound or a salt or prodrug thereof and a pharmaceutically acceptable 3-hydroxy-3-methylglutaryl coenzyme A reductase inhibitor or a salt or prodrug thereof, articles and kits and delivery devices containing such compositions, and tablets and capsules and formulations comprising such compositions. The invention also relates to methods of using such compositions to treat subjects suffering from or at risk for various diseases, disorders, and conditions, including impaired glucose tolerance; impaired fasting glucose; diabetes, including type 1 and type 2 diabetes and their complications; insulin resistance; Syndrome X; obesity and other weight related disorders; cardiomyopathy, including diabetic cardiomyopathy; atherosclerosis; coronary heart disease; hyperglycemia, hypercholesterolemia (e.g., elevated cholesterol in low-density lipoprotein (LDL-C)), hypertension, hyperinsulinemia, and/or hyperlipidemia; diseases and disorders characterized in part by any one or more of hyperlipidemia, hypercholesterolemia (e.g., elevated cholesterol in low-density lipoprotein (LDL-C)), hyperglycermia, hypertension, and/or hyperinsulinemia; diseases, disorders or conditions characterized in whole or in part by copper-related tissue damage and/or elevated LDL-C; and, diseases, disorders or conditions characterized in whole or in part by (a) hypercupremia and/or copper-related tissue damage and (b) hyperglycemia, insulin resistance, impaired glucose tolerance, and/or impaired fasting glucose, and/or elevated or undesired levels of LDL-C, or predisposition to, or risk for, (a) and (b).

### BACKGROUND

The following includes information that may be useful in understanding the present inventions. It is not an admission that any of the information provided herein is prior art, or relevant, to the presently described or claimed inventions, or that any publication or document that is specifically or implicitly referenced is prior art.

One of the key risk factors for the development of atherosclerosis is high cholesterol. Cholesterol is a fatty substance that is a normal component of the cells of the body. Cholesterol (measured in milligrams per deciliter of blood) is carried through the blood by particles known as lipoproteins, which are classified by their densities. The cholesterol in low-density lipoprotein (LDL-C) is called the “bad” cholesterol, and high levels of LDL-C increase the risk for coronary heart disease. Cholesterol in high-density lipoprotein (HDL-C) is called the “good” cholesterol, and high levels of HDL-C decrease the risk for heart attack. High levels of triglycerides, fatty acids that the body uses to store energy that has been made by the body or obtained from food, also may be associated with increased heart attack risk.

High-fat diets are associated with increased cholesterol and increased risk for heart disease. When diet and exercise alone are insufficient, drug therapy may help. The most effective and widely tested cholesterol drugs are called the statins, which block the formation of cholesterol in the liver and increase the production of the receptors on liver cells that clean the bad cholesterol from the blood. Five of these drugs are available in the United States: simvastatin, atorvastatin, lovastatin, pravastatin, fluvastatin, and rosuvastatin. Other statins include itavastatin, currently in phase 3 trials in Europe and Japan, and visastatin.

Statins can reduce LDL-C by about 20% to 45%, depending on the dosage and drug, and can decrease the risk for heart attack in men and women with both average and high levels of cholesterol, with low levels of the good cholesterol, and before and after a heart attack. In patients who have had a heart attack, the risk for dying and the risk for stroke are also decreased.

However, like many drugs, statins may be associated with potentially serious side effects. When a statin is given at a high dose, there is a risk of 1% to 2% per year for developing abnormalities in liver tests. A rarer side effect, occurring in somewhere between 1 in 1000 and 1 in 2000 patients, is inflammation of the muscles, called myopathy, in which an enzyme from the muscle leaks into the blood. The statin called cerivastatin was withdrawn from the U.S. market in August 2001 because an extreme form of this rare side effect, called rhabdomyolysis, was seen to occur more often in patients receiving cerivastatin. See Gotto, A M, “Statins: Powerful Drugs for Lowering Cholesterol,” Circulation 105:1514 (2002).

Diabetes mellitus is a group of metabolic disorders, associated with raised plasma glucose concentration and disturbance of glucose metabolism, which results in hyperglycemia. The World Health Organization (WHO) has set forth a classification scheme for diabetes mellitus that includes type 1 diabetes mellitus, type 2 diabetes mellitus, gestational diabetes, and other specific types of diabetes mellitus.

Type 1 diabetes, also known as insulin-dependent diabetes mellitus, usually develops in children or young adults. Type 1 diabetes occurs when the pancreas produces too little insulin to regulate blood sugar levels appropriately.

Although there is no set age, type 2 diabetes mellitus usually develops after 40 years of age and is much more common than type 1 diabetes, comprising approximately 90% of all individuals with diabetes. Type 2 diabetes mellitus is characterized by two different conditions: a decreased ability of insulin to act on peripheral tissues, usually referred to as “insulin resistance” and dysfunction of pancreatic B-cells, represented by the inability to produce sufficient amounts of insulin to overcome insulin resistance in the peripheral tissues. Eventually, insulin production becomes insufficient to compensate for the insulin resistance due to B-cell dysfunction. The result is a relative or absolute deficiency of insulin.

In 2001, diabetes was the sixth leading cause of death in the United States. It is estimated that about 18 million people in the United States have diabetes, and over 5 million of these people are unaware that they have the disease. The Center for Disease Control (CDC) predicts that one in three Americans born in 2000 will develop diabetes during their lifetime. The total annual economic cost of diabetes in 2002 was estimated to be $132 billion, or one out of every 10 health care dollars spent in the United States. Center for Disease Control, The Burden of Chronic Diseases and Their Risk Factors (2004). The number of people with diabetes worldwide continues to increase at alarming rates. In 1985, it was estimated that 30 million people had diabetes. In 2000 the number was increased to 171 million. By 2030 the number of people suffering from diabetes worldwide is expected to reach 366 million. Wild et al., Diabetes Care 27(5):1047-1053 (2004). Patients with diabetes have an increased incidence of long-term complications, which include atherosclerotic, cardiovascular, peripheral vascular, and cerebrovascular disease. See American Diabetes Association, Diabetes Care 16:72-78 (1993). Principal risk factors for vascular complications have been discussed in relation to the degree and duration of hyperglycemia. The Diabetes Control and Complications Trial Research Group, N Engl J Med 329:977-986 (1993). Vascular complications can be divided into two groups: microvascular and macrovascular, and include cardiovascular complications. In general, microvascular complications are said to affect the retina, kidney and nerves, while macrovascular complications are said to include diseases of the large vessels supplying the legs (lower extremity arterial disease), and predominantly the coronary, cerebrovascular and peripheral arterial circulation. Chronic hyperglycemia of diabetes is associated with long-term damage, dysfunction, and failure of various organs, especially the eyes, kidneys, nerves, heart, and blood vessels and long-term complications of diabetes include retinopathy with potential loss of vision; nephropathy leading to renal failure; peripheral neuropathy with risk of foot ulcers, amputation, and Charcot joints; and autonomic neuropathy causing gastrointestinal, genitourinary, and cardiovascular symptoms and sexual dysfunction.

Insulin resistance is a common factor in leading to hyperglycemia in type 2 diabetes. It has also been reported that impaired glucose tolerance carries an increased cardiovascular risk despite minimal hyperglycemia. Fuller J H, et al., Lancet 1:1373-1376 (1980). In the absence of diabetes, insulin resistance is reportedly a major risk factor for CAD. Lempiainen P, et al., Circulation 100:123-128 (1999).

Insulin resistance coupled with compensatory hyperinsulinemia leads to a number of proatherogenic abnormalities referred to as Insulin Resistance Syndrome. Insulin Resistance Syndrome (or Syndrome X) is a constellation of metabolic disturbances, which enhance cardiovascular risk. Syndrome characteristics include deposition of fat around the abdominal organs, called visceral or central adiposity; changes in the lipoprotein profile, such as decrease in HDL, a rise in triglycerides; and, increased LDL. An increase in blood pressure is seen in many, but not all, insulin resistant populations. Increased fibrinogen, a clotting and inflammatory marker, and PAI-1, are also reported.

Current treatment for hyperglycemia includes the administration of hyperglycemic agents, such as sulfonylureas, biguanides, alpha-glucosidase inhibitors, meglitinides, thiazolidinediones, and D-phenylalanine derivatives. These drugs reduce glucose levels by a variety of different methods, including lowering sugar absorption, increasing insulin production, and increasing insulin sensitivity. Current treatment for hypercholesterolemia, including elevated or undesired levels of LDL-C, includes the administration of 3-hydroxy-3-methylglutaryl coenzyme A reductase inhibitors, such as the statins. Commonly used 3-hydroxy-3-methylglutaryl coenzyme A reductase inhibitors include atorvastatin and simvastatin.

Heart disease is the leading cause of death for both women and men in the United States. In 2001, 700,142 people died of heart disease (52% of them women), accounting for 29% of all U.S. deaths. The age-adjusted death rate was 246 per 100,000 population. In 2001, heart disease cost the United States $193.8 billion in total health care costs. The burden of heart disease could be ameliorated by reducing the prevalence rates of its major risk factors: high blood pressure, high blood cholesterol, tobacco use, diabetes, physical inactivity, and poor nutrition. Modest reductions in the rates of one or more of these risk factors can have a large public health impact. Center for Disease Control, The Burden of Chronic Diseases and Their Risk Factors (2004).

Metal ions are essential for cells, but can become toxic at higher concentrations, and free metal ions have been implicated in heart disease. Metal ions replace other essential metals in enzymes or molecules, which can disrupt their function. Metal ions such as Hg⁺ and Cu+ are reactive to thiol groups and may interfere with protein structure and function. Redox active transition metals such as Fe2+/3+ and Cu+/2+, which can take up or give off an electron, may give rise to free radicals which can cause oxidative stress. Jones et al., Biochim. Biophys. Acta 286: 652-655 (1991); Li and Trush, Carcinogenes 7: 1303-1311 (1993).

Wilson's disease is due to a defect in copper excretion into the bile by the liver. Also known as hepatolenticular degeneration, Wilson's disease occurs in individuals who have inherited an autosomal recessive defect that leads to an accumulation of copper in excess of metabolic requirements. The excess copper is deposited in several organs and tissues, and eventually produces pathological effects primarily in the liver, where damage progresses to postnecrotic cirrhosis, and in the brain, where degeneration is widespread. Copper is also deposited as characteristic, asymptomatic, golden-brown Kayser-Fleisher rings in the corneas of all patients with cerebral symptomatology and some patients who are either asymptomatic or manifest only hepatic symptomatology. Wilson's disease generally affects patients between the ages of 10 and 40 years.

Wilson's disease is generally treated with an orally administered copper chelator. First line therapy for treatment of Wilson's disease is penicillamine, a chelating agent. Penicillamine, 3-mercapto-D-valine, is also used to reduce cystine excretion in cystinuria and to treat patients with severe, active rheumatoid arthritis unresponsive to conventional therapy. It is a white or practically white, crystalline powder, freely soluble in water, slightly soluble in alcohol, and insoluble in ether, acetone, benzene, and carbon tetrachloride. Although its configuration is D₁ it is levorotatory as usually measured. The empirical formula is C₅H₁₁NO₂S, giving it a molecular weight of 149.21. It reacts readily with formaldehyde or acetone to form a thiazolidine-carboxylic acid. Cuprimine® (Penicillamine) capsules for oral administration contain either 125 mg or 250 mg of penicillamine, as well as D & C Yellow 10, gelatin, lactose, magnesium stearate, and titanium dioxide as inactive ingredients. The 125 mg capsule also contains iron oxide for capsule color.

Trientine, a chelating compound for removal of excess copper from the body, is prescribed for Wilson's disease patients who cannot tolerate penicillamine. Trientine hydrochloride is N,N′-bis(2-aminoethyl)-1,2-ethanediamine dihydrochloride. It is a white to pale yellow crystalline hygroscopic powder. It is freely soluble in water, soluble in methanol, slightly soluble in ethanol, and insoluble in chloroform and ether. The empirical formula is C₆H₁₈N₄.2HCl and it has a molecular weight of 2119.2. The structural formula is: NH₂(CH₂)₂—NH(CH₂)₂—NH(CH₂)₂—NH₂.2HCl. Syprine® (trientine hydrochloride) is available as 250 mg capsules for oral administration. Syprine® capsules reportedly contain gelatin, iron oxides (for capsule color), stearic acid, and titanium dioxide as inactive ingredients. It has been reported that chelated copper in patients with Wilson's disease is excreted primarily through the feces, either by the effective chelation of copper in the gut, or by partial restoration of mechanisms that allow for excretion of excess copper via urine or into the bile, or a combination of the two. See Siegemund R, et al., “Mode of action of triethylenetetramine dihydrochloride on copper metabolism in Wilson's disease,” Acta Neurol Scand. 83(6):364-6 (June 1991).

Zinc acetate (Galzin™) blocks the absorption of copper in the intestinal tract and was recently approved by the FDA for treatment of Wilson's disease. By blocking copper absorption, newly ingested copper does not reach the circulation and is excreted mainly in the stool. Zinc acetate has not shown any long-term or major side effects in patients and can be used, long-term, in place of non-tolerable chelating agents. This is useful for patients who develop adverse reactions to chelating agents.

U.S. Pat. Nos. 6,610,693, 6,348,465 and 6,897,243 provide copper chelators and other agents (e.g., zinc which prevents copper absorption) to decrease copper values for the benefit of subjects suffering from diabetes and its complications. See also, Cooper, G. J., et al., “Regeneration of the heart in diabetes mellitus by selective copper chelation,” Diabetes 53:2501-2508 (2004); Cooper, G. J., et al., “Preventing and/or treating cardiovascular disease and/or associated heart failure,” U.S. Pat. No. 6,951,890.

Despite correlations between heart disease and hypercholesterolemia in diabetic and other patients, these conditions are treated separately using different drugs and drug forms. Compositions and methods of the invention that employ 3-hydroxy-3-methylglutaryl coenzyme A reductase inhibitors in combination with copper antagonist agents, for example, copper (II) antagonists are disclosed and claimed. These combinations also, for example, allow the use of lower doses of each agent than previously required to achieve desired therapeutic goals.

### BRIEF SUMMARY

The inventions described and claimed herein have many attributes and embodiments including, but not limited to, those set forth or described or referenced in this Brief Summary. It is not intended to be all-inclusive and the inventions described and claimed herein are not limited to or by the features or embodiments identified in this Brief Summary, which is included for purposes of illustration only and not restriction.

The invention includes pharmaceutical compositions comprising (a) a therapeutically effect amount of a pharmaceutically acceptable copper antagonist or a pharmaceutically acceptable salt, for example, an acid addition salt, or prodrug, thereof; (b) a therapeutically effect amount of a 3-hydroxy-3-methylglutaryl coenzyme A reductase inhibitor or a pharmaceutically acceptable salt thereof, for example, an acid addition salt; and, (c) a pharmaceutically acceptable carrier or diluent.

Suitable copper antagonists include pharmaceutically acceptable copper chelators. Cu²⁺ antagonists, for example, Cu²⁺ chelators, are preferred. Copper antagonists may be present in the compositions of the invention in an amount, for example, that is effective to (1) increase copper output in the urine of said subject, (2) decrease body and/or tissue copper levels, (3) decrease copper uptake, for example, in the gastrointestinal tract, (4) decrease SOD, for example, EC-SOD, as measured by mass or activity, (6) decrease homocysteine, (7) decrease oxidative stress and/or (8) increase copper (I).

Copper antagonists useful in the invention include, but are not limited to, pharmaceutically acceptable compounds of Formulae I, I(a) and II herein. Other suitable copper antagonists include, for example, pharmaceutically acceptable linear or branched tetramines capable of binding copper; 2,3,2 tetramine and salts thereof; 2,2,2 tetramine (also referred to as trientine) and salts thereof; 3,3,3 tetramine and salts thereof; triethylenetetramine hydrochloride salts, for example, triethylenetetramine dihydrochloride and triethylenetetramine tetrahydrochloride; triethylenetetramine succinate salts, for example, triethylenetetramine disuccinate, triethylenetetramine maleate salts, for example, triethylenetetramine tetramaleate and triethylenetetramine tetramaleate dihydrate; and triethylenetetramine fumarate salts, for example, triethylenetetramine tetrafumarate and triethylenetetramine tetrafumarate tetrahydrate.

According to one aspect, suitable copper antagonist salts include a salt of a compound of Formula I (a) and a pharmaceutically acceptable dicarboxylic organic acid or tricarboxylic organic acid. Suitable dicarboxylic organic acids include aliphatic dicarboxylic acids. Such dicarboxylic acids include an aliphatic dicarboxylic acid of the formula HOOC-Q₁-COOH wherein Q₁ is alkylene of 1 to about 8 carbon atoms or alkenylene of 2 to about 8 carbon atoms and includes both straight chain and branched chain alkylene and alkenylene groups. Examples of dicarboxylic organic acids and tricarboxylic organic acids include oxalic acid, malonic acid, succinic acid, glutaric acid, adipic acid, pimelic acid, suberic acid, azelaic acid, sebacic acid, maleic acid, fumaric acid, citraconic acid, mesoconic acid, itaconic acid, tricarballytic acid, 1,2,3-butanetricarboxylic acid, trimesic acid, hemimellitic acid, and trimellitic acid. Certain salts are described in Provisional U.S. Patent Application No. 60/772,451 filed Feb. 9, 2006, the disclosure of which is incorporated herein by reference.

Other suitable copper antagonists include, for example, crystalline triethylenetetramine and salts thereof. These include crystalline triethylenetetramine maleate (e.g., triethylenetetramine tetramaleate and triethylenetetramine tetramaleate dihydrate), crystalline triethylenetetramine fumarate (e.g., triethylenetetramine tetrafiumarate and triethylenetetramine tetrafumarate tetrahydrate), and crystalline triethylenetetramine succinate (e.g., triethylenetetramine disuccinate anhydrate).

Other agents capable of reducing copper include thiomolybdates (including mono-, di-, tri- and tetrathiomolybdates); zinc salts, such as zinc acetate; zinc chloride; zinc sulphate; zinc salts of intermediates of the citric acid cycle, such as citrate, isocitrate, ketoglutarate, succinate, malate; and, zinc glucoante.

Copper antagonists useful in the invention also include copper antagonizing metabolites, such as copper antagonizing metabolites of trientine including, for example, N-acetyl trientine, and analogues, derivatives, and prodrugs thereof. Copper antagonists useful in the invention also include modified copper antagonists, for example, modified trientines. Derivatives of copper antagonists, including trientine or trientine salts or analogues, include those modified with polyethylene glycol (PEG).

Copper antagonists useful in the invention also include copper antagonists, including copper chelators, which have been pre-complexed with a non-copper metal ion prior to administration for therapy, the non-copper metal ion having a binding affinity for the copper antagonist that is lower that that of copper (e.g., lower than that of Cu²⁺).

Also encompassed are metal complexes comprising copper antagonists and non-copper metals (that have lower binding affinities than copper for the copper antagonist) and one or more additional ligands than typically found in complexes of that metal. These include, for example, pentacoordinate copper complexes of triethylenetetramine and another ligand.

Suitable 3-hydroxy-3-methylglutaryl coenzyme A reductase inhibitors include the statins. Preferred statins are simvastatin, atorvastatin, lovastatin, pravastatin, fluvastatin, and rosuvastatin. Other statins include itavastatin and visastatin. Such compounds are present in the compositions of the invention amounts, for example, that are effective to lower LDL-C.

Suitable copper antagonist salts include acid addition salts such as, for example, those of suitable inorganic or organic acids. Suitable organic acids include succinic acid, maleic acid, and fumaric acid. Suitable inorganic acids include hydrochloric acid. Succinate salts are preferred. Triethylenetetramine discuccinate is most preferred.

The invention includes pharmaceutical compositions, including tablets and capsules and other oral delivery forms and formulations, comprising a pharmaceutically acceptable carrier and therapeutically effective amounts of a 3-hydroxy-3-methylglutaryl coenzyme A reductase inhibitor and one or more compounds of Formulae I, I(a) and II herein.

The invention includes pharmaceutical compositions, including tablets and capsules and other oral delivery forms and formulations, comprising a pharmaceutically acceptable carrier and therapeutically effective amounts of a 3-hydroxy-3-methylglutaryl coenzyme A reductase inhibitor and one or more linear or branched tetramines capable of binding copper. Examples of tetramines include 2,3,2 tetramine, 2,2,2 tetramine, and 3,3,3 tetramine, and salts thereof.

The invention includes pharmaceutical compositions, including tablets and capsules and other oral delivery forms and formulations, comprising a pharmaceutically acceptable carrier and therapeutically effective amounts of a 3-hydroxy-3-methylglutaryl coenzyme A reductase inhibitor and triethylenetetramine or a triethylenetetramine salt(s).

The invention includes pharmaceutical compositions, including tablets and capsules and other oral delivery forms and formulations, comprising a pharmaceutically acceptable carrier and therapeutically effective amounts of a 3-hydroxy-3-methylglutaryl coenzyme A reductase inhibitor and one or more triethylenetetramine hydrochloride salts, for example, triethylenetetramine dihydrochloride and triethylenetetramine tetrahydrochloride.

The invention includes pharmaceutical compositions, including tablets and capsules and other oral delivery forms and formulations, comprising a pharmaceutically acceptable carrier and therapeutically effective amounts of a 3-hydroxy-3-methylglutaryl coenzyme A reductase inhibitor and one or more triethylenetetramine succinate salts, for example, triethylenetetramine disuccinate.

The invention includes pharmaceutical compositions, including tablets and capsules and other oral delivery forms and formulations, comprising a pharmaceutically acceptable carrier and therapeutically effective amounts of a 3-hydroxy-3-methylglutaryl coenzyme A reductase inhibitor and one or more triethylenetetramine maleate salts, for example, triethylenetetramine tetramaleate and triethylenetetramine tetramaleate dihydrate; or triethylenetetramine fumarate salts, for example, triethylenetetramine tetrafumarate and triethylenetetramine tetrafumarate tetrahydrate.

The invention includes pharmaceutical compositions, including tablets and capsules and other oral delivery forms and formulations, comprising a pharmaceutically acceptable carrier and therapeutically effective amounts of a copper antagonist and 3-hydroxy-3-methylglutaryl coenzyme A reductase inhibitor, for example, a statin. Statins include, for example, simvastatin, atorvastatin, lovastatin, pravastatin, and fluvastatin.

The invention includes methods for treating and/or preventing, in whole or in part, various diseases, disorders and conditions, including, for example, atherosclerosis; coronary heart disease; impaired glucose tolerance; impaired fasting glucose; diabetes and/or its complications, including type 1 and type 2 diabetes and their complications; insulin resistance; Syndrome X; obesity and other weight related disorders; cardiomyopathy, including diabetic cardiomyopathy; hyperglycemia, hypercholesterolemia (e.g., elevated cholesterol in low-density lipoprotein (LDL-C)), hypertension, hyperinsulinemia, and/or hyperlipidemia; diseases, disorders and conditions characterized in part by any one or more of hyperlipidemia, hypercholesterolemia, hyperglycemia, hypertension, and/or hyperinsulinemia; and, diseases, disorders or conditions characterized in whole or in part by (a) hypercupremia and/or copper-related tissue damage and (b) hyperglycemia, insulin resistance, impaired glucose tolerance, and/or impaired fasting glucose, and/or elevated or undesired levels of LDL-C, or predisposition to, or risk for, (a) and (b).

The invention includes methods for treating a subject having or suspected of having or predisposed to, or at risk for, for example, any diseases, disorders and/or conditions characterized in whole or in part, for example, by (a) hypercupremia and/or copper-related tissue damage and (b) hyperglycemia, insulin resistance, impaired glucose tolerance, impaired fasting glucose and/or elevated or undesired levels of LDL-C, comprising administering a composition comprising a pharmaceutically acceptable copper antagonist and a 3-hydroxy-3-methylglutaryl coenzyme A reductase inhibitor. Such diseases, disorders and/or conditions include but are not limited to those described or referenced herein. Such compounds may be administered in amounts, for example, that are effective to (1) decrease body and/or tissue copper levels, (2) increase copper output in the urine of said subject, (3) decrease copper uptake, for example, in the gastrointestinal tract, (4) decrease SOD, for example, EC-SOD, as measured by mass or activity, (5) decrease homocysteine, (6) decrease oxidative stress (7) increase copper (I), and/or (8) lower LDL-C. Such compositions include, for example, tablets and capsules and other oral delivery forms and formulations.

The invention includes methods for regulating LDL-C and diminishing copper and/or available copper in a subject having, suspected of having, at risk for, or predisposed to diseases, disorders and/or conditions characterized in whole or in part by (a) hypercupremia and/or copper-related tissue damage and (b) hyperglycemia, insulin resisance, impaired glucose tolerance, impaired fasting glucose and/or elevated or undesired levels of LDL-C, comprising administering a composition comprising a pharmaceutically acceptable copper antagonist and a 3-hydroxy-3-methylglutaryl coenzyme A reductase inhibitor. Such diseases, disorders and/or conditions include but are not limited to those described or referenced herein. Such compounds may be administered in amounts, for example, that are effective to (1) decrease body and/or tissue copper levels, (2) increase copper output in the urine of said subject, (3) decrease copper uptake, for example, in the gastrointestinal tract, (4) decrease SOD, for example, EC-SOD, as measured by mass or activity, (5) decrease homocysteine, (6) decrease oxidative stress (7) increase copper (I), and/or (8) lower LDL-C. Such compositions include, for example, tablets and capsules and other oral delivery forms and formulations.

The invention includes methods for administering a therapeutically effective amount of a pharmaceutically acceptable copper antagonist and a 3-hydroxy-3-methylglutaryl coenzyme A reductase inhibitor formulated in a delayed release preparation, a slow release preparation, an extended release preparation, a controlled release preparation, and/or in a repeat action preparation to a subject having or suspected of having or predisposed to diseases, disorders and/or conditions characterized in whole or in part by (a) hypercupremia and/or copper-related tissue damage and (b) hyperglycemia, insulin resistance, impaired glucose tolerance, impaired fasting glucose, and/or elevated or undesired levels of LDL-C, comprising administering a composition comprising a pharmaceutically acceptable copper antagonist and a 3-hydroxy-3-methylglutaryl coenzyme A reductase inhibitor. Such diseases, disorders and conditions include, but are not limited to, those herein disclosed herein. Such compounds may be administered in amounts, for example, that are effective to (1) decrease body and/or tissue copper levels, (2) increase copper output in the urine of said subject, (3) decrease copper uptake, for example, in the gastrointestinal tract, (4) decrease SOD, for example, EC-SOD, as measured by mass or activity, (5) decrease homocysteine, (6) decrease oxidative stress (7) increase copper. (I), and/or (8) lower LDL-C. Such compositions include, for example, tablets and capsules and other oral delivery forms and formulations.

The invention includes methods for the use of therapeutically effective amounts of a pharmaceutically acceptable copper antagonist and a pharmaceutically acceptable 3-hydroxy-3-methylglutaryl coenzyme A reductase inhibitor in the manufacture of a medicament. Such medicaments include, for example, tablets and capsules and other oral delivery forms and formulations. Such medicaments include those for the treatment of a subject as disclosed herein.

The invention includes methods for the use of a therapeutically effective amount of a copper antagonist and a pharmaceutically acceptable 3-hydroxy-3-methylglutaryl coenzyme A reductase inhibitor in the manufacture of a dosage form. Such dosage forms include, for example, tablets and capsules and other oral delivery forms and formulations. Such dosage forms include those for the treatment of a subject as disclosed herein.

The invention includes transdermal patches, pads, wraps, and bandages capable of being adhered or otherwise associated with the skin of a subject, said articles being capable of delivering a therapeutically effective amount of a pharmaceutically acceptable copper antagonist and a pharmaceutically acceptable 3-hydroxy-3-methylglutaryl coenzyme A reductase inhibitor to a subject.

The invention includes an article of manufacture comprising a vessel containing a therapeutically effective amount of a pharmaceutically acceptable copper antagonist and a pharmaceutically acceptable 3-hydroxy-3-methylglutaryl coenzyme A reductase inhibitor and instructions for use, including use for the treatment of a subject.

The invention includes an article of manufacture comprising packaging material containing one or more dosage forms containing a pharmaceutically acceptable copper antagonist and a pharmaceutically acceptable 3-hydroxy-3-methylglutaryl coenzyme A reductase inhibitor, wherein the packaging material has a label that indicates that the dosage form can be used for a subject having or suspected of having or predisposed to any of the diseases, disorders and/or conditions described or referenced herein, including diseases, disorders and/or conditions characterized in whole or in part by elevated or undesired levels of LDL-C and/or hypercupremia, including but not limited to those herein disclosed herein. Such dosage forms include, for example, tablets and capsules and other oral delivery forms and formulations.

The invention includes a formulation comprising a pharmaceutically acceptable copper antagonist and a pharmaceutically acceptable 3-hydroxy-3-methylglutaryl coenzyme A reductase inhibitor in amounts effective to remove copper from the body of a subject and reduce elevated or undesired levels of LDL-C in the subject. Such formulations include, for example, tablets and capsules and other oral delivery forms and formulations.

The invention includes devices containing therapeutically effective amounts of a pharmaceutically acceptable copper antagonist and a pharmaceutically acceptable 3-hydroxy-3-methylglutaryl coenzyme A reductase inhibitor, for example, a rate-controlling membrane enclosing a drug reservoir and a monolithic matrix device. These devices may be employed for the treatment of subjects in need thereof as disclosed herein.

These and other aspects of the inventions, which are not limited to or by the information provided in this Brief Summary, are discussed below.

### DETAILED DESCRIPTION

As used herein, a “copper antagonist” is a pharmaceutically acceptable compound that binds or chelates copper, preferably copper (II), in vivo for removal. Copper chelators are presently preferred copper antagonists. Copper (II) chelators, and copper (II) specific chelators (i.e., those that preferentially bind copper (II) over other forms of copper such as copper (I)), are especially preferred. “Copper (II)” refers to the oxidized (or +2) form of copper, also sometimes referred to as Cu⁺².

As used herein, a “disorder” is any disorder, disease, or condition that would benefit from an agent that reduces local or systemic copper, extracellular copper, bound copper, or copper concentrations, and/or an agent that reduces LDL-C, for example. Particularly preferred are agents that reduce extracellular copper or extracellular copper concentrations (local or systemic) and, more particularly, agents that reduce extracellular copper (II) or extracellular copper (II) concentrations (local or systemic). Particularly preferred agents that reduce LDL-C are 3-hydroxy-3-methylglutaryl coenzyme A reductase inhibitors, which include the statins. Disorders include, but are not limited to, those described and/or referenced herein, and include diseases, disorders and conditions include that would benefit from (1) a decrease body and/or tissue copper levels, (2) an increase copper output in the urine of said subject, (3) a decrease copper uptake, for example, in the gastrointestinal tract, (4) decrease SOD, for example, EC-SOD, as measured by mass or activity, (5) decrease homocysteine, (6) decrease oxidative stress (7) increase copper (I), and/or (8) lower LDL-C.

As used herein, “3-hydroxy-3-methylglutaryl coenzyme A reductase inhibitor” refers to pharmaceutically acceptable therapeutic compounds capable blocking or inhibiting 3-hydroxy-3-methylglutaryl coenzyme A. 3-hydroxy-3-methylglutaryl coenzyme A reductase inhibitors include statins, including simvastatin, atorvastatin, lovastatin, pravastatin, fluvastatin, and rosuvastatin. Other statins include itavastatin and visastatin.

As used herein, “mammal” refers to any animal classified as a mammal, including humans, domestic and farm animals, and zoo, sports, or pet animals, such as dogs, horses, cats, sheep, pigs, cows, etc. The preferred mammal herein is a human.

As used herein, “pharmaceutically acceptable salts” refers to salts prepared from pharmaceutically acceptable non-toxic bases or acids including inorganic or organic bases and inorganic or organic acids the like. When a copper antagonist compound is basic, for example, salts may be prepared from pharmaceutically acceptable non-toxic acids, including inorganic and organic acids. Organic acids include both aliphatic and aromatic carboxylic acids and include, for example, aliphatic monocarboxylic acids, aliphatic dicarboxylic acids, aliphatic tricarboxylic acids, aromatic monocarboxylic acids, aromatic dicarboxylic acids, aromatic tricarboxylic acids and other organic acids known to those of skill in the art. Aliphatic carboxylic acids may be saturated or unsaturated. Suitable aliphatic carboxylic acids include those having from 2 to about 10 carbon atoms. Aliphatic monocarboxylic acids include saturated aliphatic monocarboxylic acids and unsaturated aliphatic monocarboxylic acids. Examples of saturated monocarboxylic acids include acetic acid, propronic acid, butyric acid, valeric acid, caproic acid, enanthic acid, caprylic acid, pelargonic acid, and caprynic acid. Examples of unsaturated aliphatic monocarboxylic acids include acrylic acid, propiolic acid, methacrylic acid, crotonic acid and isocrotonic acid. Aliphatic dicarboxylic acids include saturated aliphatic dicarboxylic acids and unsaturated aliphatic dicarboxylic acids. Examples of saturated aliphatic dicarboxylic acids include oxalic acid, malonic acid, succinic acid, glutaric acid, adipic acid, pimelic acid, suberic acid, azelaic acid, and sebacic acid. Examples of unsaturated aliphatic dicarboxylic acids include maleic acid, fumaric acid, citraconic acid, mesaconic acid, itaconic acid and the like. Aliphatic tricarboxylic acids includes saturated aliphatic tricarboxylic acids and unsaturated tricarboxylic acids. Examples of saturated tricarboxylic acids include tricarballylic acid, 1,2,3-butanetricarboxylic acid and the like. Suitable aliphatic dicarboxylic acids include those of the formula: HOOC-Q₁-COOH, wherein Q₁ is alkylene of 1 to about 8 carbon atoms or alkenylene of 2 to about 8 atoms, and includes both straight chain and branched chain alkylene and alkenylene groups. Examples of aromatic dicarboxylic acids include phthalic acid, isophthalic acid, terephthalic acid and the like. Examples of aromatic tricarboxylic acids include trimesic acid, hemimellitic acid and trimellitic acid. Such acids may also include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are hydrochloric, maleic, fumaric and succinic acid copper antagonist salts. Succinic acid copper antagonist salts are most preferred, particularly for those copper antagonist salts that are not anhydrous.

As used herein, “preventing” means preventing in whole or in part, or ameliorating or controlling.

As used herein, a “therapeutically effective amount” in reference to the compounds or compositions of the instant invention refers to the amount sufficient to induce a desired biological, pharmaceutical, or therapeutic result. That result can be alleviation of the signs, symptoms, or causes of a disease or disorder or condition, or any other desired alteration of a biological system. In the present invention, the result will involve the prevention, decrease, or reversal of tissue injury, in whole or in part, and reduced LDL-C, as referenced herein. Therapeutic effects include, for example, (1) decreasing body and/or tissue copper levels, (2) increasing copper output in the urine, (3) decreasing copper uptake, for example, in the gastrointestinal tract, (4) decrease SOD, for example, EC-SOD, as measured by mass or activity, (5) decrease homocysteine, (6) decrease oxidative stress (7) increase copper (I), and/or (8) lowering LDL-C.

As used herein, the term “treating” refers to both therapeutic treatment and prophylactic or preventative measures. Those in need of treatment include those already with the disorder as well as those prone to having the disorder or diagnosed with the disorder or those in which the disorder is to be prevented.

A reduction in copper, particularly extracellular copper that is generally in the its copper II form, will be advantageous in the treatment of disorders, diseases, and/or conditions, caused or exacerbated by mechanisms that may be affected by or are dependent on excess copper values and/or hyperglycemia. For example, a reduction in copper and/or LDL-C will be advantageous in providing a combined reduction in and/or reversal of copper-associated and/or LDL-C-associated damage.

Copper antagonist/3-hydroxy-3-methylglutaryl coenzyme A reductase inhibitor combinations may be prepared for administration via oral delivery. The preparation of various tablets and capsules are described in Examples 1-24. They include tablets (see, e.g., Examples 1 and 13), tablets with a filler(s) (see, e.g., Examples 2 and 14), tablets with a desiccant(s) (see, e.g., Examples 3 and 15), tablets with a wet granulations binder(s) (see, e.g., Examples 4 and 16), tablets with a wet granulations binder(s) and a desiccant(s) (see, e.g., Examples 5 and 17), capsules (see, e.g., Examples 6 and 18), capsules with a desiccant(s) (see, e.g., Examples 7 and 19), capsules with a filler(s) (see, e.g., Examples 8 and 20), capsules with a filler(s) and a granulation binder(s) (see, e.g., Examples 9 and 21), capsules with a desiccant(s) and a granulation binder(s) (see, e.g., Examples 10 and 22), controlled release tablets (see, e.g., Examples 11 and 23), and, capsules with enteric coated beads (see, e.g., Examples 12 and 24). Examples 1-24 relate to simvastatin and atorvastatin, but other statins may be used and included in amounts depending on desired dose and frequency of administration, including doses lower than those presently prescribed for lowering LDL-C. Where triethylenetetramine dihydrochloride is included in wet formulations it is preferably precomplexed with a non-copper metal ion as disclosed herein, e.g., calcium, to enhance stability.

Statins may be prepared using art-known methods, including, for example, the methods described in U.S. Pat. Nos. 6,838,566 and 6,825,015. For example, simvastatin may be prepared as described in U.S. Pat. Nos. 4,444,784, 4,582,915, 4,820,850, 4,450,171, 5,763,646 and 6,696,086 and is administered as known in the art (see, for example, U.S. Pat. Nos. 4,444,784, 5,846,966, 5,622,985, and RE37721). Simvastatin may be prepared for administration in a dose in the range of about 1 mg to about 2000 mg/day, including from about 4 mg to about 200 mg per dose, in an adult patient of about 70 kg body weight. Dosage forms, for example, tablets and capsules may be prepared accordingly, or using other doses as disclosed herein, and lower doses than those presently prescribed for lowering LDL-C may be used.

Atorvastatin may be prepared as described in U.S. Pat. Nos. 4,681,893, 5,273,995, 5,385,929, 4,879,303 and 5,686,104, and is administered as known in the art (see, for example, U.S. Pat. Nos. 4,572,909, 4,681,893, 5,686,104, 5,969,156, 6,126,971, and 6,455,574). Atorvastatin may be prepared for administration in a dose in the range of about 10 mg to about 500 mg, including from about 20 to about 100 mg per dose, in an adult patient of about 70 kg body weight. Dosage forms, for example, tablets and capsules may be prepared accordingly, or using other doses as disclosed herein, and lower doses than those presently prescribed for lowering LDL-C may be used.

Mevastatin may be prepared using methods described in U.S. Patent Nos. 3,983,140 and 3,671,523 or alternatively, may purchased in bulk from manufacturers (e.g., Haorui Pharma-Chem Inc., New Jersey USA. Administration of mevastatin is known in the art (see, for example, U.S. Pat. No. 3,983,140). Mevastatin may be prepared for administration in a dose in the range of about 1 mg to about 2000 mg/day, in an adult patient of about 70 kg body weight. Dosage forms, for example, tablets and capsules may be prepared accordingly, or using other doses as disclosed herein, and lower doses than those presently prescribed for lowering LDL-C may be used.

Lovastatin may be prepared as described in U.S. Pat. Nos. 4,231,938, 4,231,893, and 5,916,595 and is administered as known in the art (see, for example, U.S. Pat. Nos. 6,080,778 6,521,762, 5,622,985, and 5,595,734). Lovastatin may be prepared for administration in a dose in the range of about 1 mg to about 2000 mg/day, including from about 4 mg to about 200 mg per dose unit, in an adult patient of about 70 kg body weight. Dosage forms, for example, tablets and capsules may be prepared accordingly, or using other doses as disclosed herein, and lower doses than those presently prescribed for lowering LDL-C may be used.

Pravastatin may be prepared as described in U.S. Pat. Nos. 4,346,227, 5,030,447, 5,180,589, 6,740,775, and 6,682,913 and is administered as known in the art (see, for example, U.S. Pat. Nos. 5,622,985 and 5,260,305). Pravastatin may be prepared for administration in a dose in the range of about 1 mg to about 2000 mg/day, including from about 4 mg to about 200 mg per dose unit, in an adult patient of about 70 kg body weight. Dosage forms, for example, tablets and capsules may be prepared accordingly, or using other doses as disclosed herein, and lower doses than those presently prescribed for lowering LDL-C may be used.

Fluvastatin may be prepared as described in U.S. Pat. Nos. 4,739,073, 5,354,772, 5,356,896, and 6,242,003 and is administered as known in the art (see, for example, U.S. Pat. Nos. 4,739,073 and 5,356,896). Fluvastatin may be prepared for administration in a dose in the range of about 0.5 mg to about 500 mg, including from about 0.5 to about 50 mg per dose, in an adult patient of about 70 kg body weight. Dosage forms, for example, tablets and capsules may be prepared accordingly, or using other doses as disclosed herein, and lower doses than those presently prescribed for lowering LDL-C may be used.

Rosuvastatin may be prepared as described in U.S. Pat. Nos. RE37,314 and 5,260,440, and is administered as known in the art (see, for example, U.S. Pat. No. 5,260,440). Rosuvastatin may be prepared for parenteral administration in a dose in the range of about 0.1 mg to about 100 mg/day, including from about 0.5 to about 50 mg/day, in an adult patient of about 70 kg body weight. For oral administration, rosuvastatin is preferably prepared in a dose in the range of about 0.5 mg to about 200 mg/day, more preferably from about 1 to about 100 mg/day, in an adult patient of about 70 kg body weight. Dosage forms, for example, tablets and capsules may be prepared accordingly, or using other doses as disclosed herein, and lower doses than those presently prescribed for lowering LDL-C may be used.

Itavastatin (Also referred to as pitavastatin) may be prepared as described in U.S. Pat. Nos. 5,011,930, 6,777,552, and 5,753,675, and is administered as known in the art (see, for example, U.S. Pat. No. 5,011,930). Itavastatin may be prepared for administration in a dose in the range of about 0.05 mg to about 500 mg/day, including from about 0.5 mg to about 50 mg/day, in an adult patient of about 70 kg body weight. For sustained release, visastatin may be prepared in a dose in the range of about 0.25 mg to about 250 mg/day, including from about 0.25 mg to about 25 mg per dose, in an adult patient of about 70 kg body weight. Dosage forms, for example, tablets and capsules may be prepared accordingly, or using other doses as disclosed herein, and lower doses than those presently prescribed for lowering LDL-C may be used.

Visastatin may be prepared as described in U.S. Pat. Nos. 55,260,440 and 6,777,552, and is administered as known in the art (see, for example, U.S. Pat. No. 5,260,440). Visastatin may be prepared for parenteral administration in a dose in the range of about 0.1 mg to about 100 mg/day, for example from about 0.5 to about 5 mg/day, in an adult patient of about 70 kg body weight. For oral administration, visastatin may be prepared in a dose in the range of about 0.5 mg to about 200 mg/day, including from about 1 to about 100 mg/day, in an adult patient of about 70 kg body weight. Dosage forms, for example, tablets and capsules may be prepared accordingly, or using other doses as disclosed herein, and lower doses than those presently prescribed for lowering LDL-C may be used.

Pharmaceutically acceptable copper antagonists, preferably copper (II) antagonists, and more preferably copper (II) chleator agents, may be used in the invention. Copper antagonists include, for example, trientine active agents, which include trientines (triethylenetetramines).

Copper antagonists useful in the invention include, but are not limited to, pharmaceutically acceptable compounds of Formulae I, I(a) and II herein. Other suitable copper antagonists include, for example, pharmaceutically acceptable linear or branched tetramines capable of binding copper; 2,3,2 tetramine and salts thereof; 2,2,2 tetramine (also referred to as trientine) and salts thereof; 3,3,3 tetramine and salts thereof; triethylenetetramine hydrochloride salts, for example, triethylenetetramine dihydrochloride and triethylenetetramine tetrahydrochloride; triethylenetetramine succinate salts, for example, triethylenetetramine disuccinate; triethylenetetramine maleate salts, for example, triethylenetetramine tetramaleate and triethylenetetramine tetramaleate dihydrate; and triethylenetetramine fumarate salts, for example, triethylenetetramine tetrafumarate and triethylenetetramine tetrafumarate tetrahydrate.

Other suitable copper antagonists include, for example, crystalline triethylenetetramine and salts thereof. These include crystalline triethylenetetramine maleate (e.g., triethylenetetramine tetramaleate and triethylenetetramine tetramaleate dihydrate), crystalline triethylenetetramine fumarate (e.g., triethylenetetramine tetrafumarate and triethylenetetramine tetrafumarate tetrahydrate), and crystalline triethylenetetramine succinate (e.g, triethylenetetramine disuccinate anhydrate).

Other agents capable of reducing copper include thiomolybdates (including mono-di-, tri- and tetrathiomolybdates); zinc salts, such as zinc acetate; zinc chloride; zinc sulphate; zinc salts of intermediates of the citric acid cycle, such as citrate, isocitrate, ketoglutarate, succinate, malate; and, zinc glucoante.

Copper antagonists useful in the invention also include copper antagonizing metabolites, such as copper antagonizing metabolites of trientine including, for example, N-acetyl trientine, and analogues, derivatives, and prodrugs thereof. Copper antagonists useful in the invention also include modified copper antagonists, for example, modified trientines. Derivatives of copper antagonists, including trientine or trientine salts or analogues, include those modified with polyethylene glycol (PEG).

The invention includes pharmaceutical compositions comprising a therapeutically effective amount of a pharmaceutically acceptable precomplexed copper antagonist or a pharmaceutically acceptable salt, for example, an acid addition salt, thereof and a pharmaceutically acceptable carrier or diluent. Thus, copper antagonists useful in the invention also include copper antagonists, including copper chelators, which have been pre-complexed with a non-copper metal ion prior to administration for therapy. Metal ions used for pre-complexing have a lower association constant for the copper antagonist than that of copper. For example, a metal ion for pre-complexing a copper antagonist that chelates Cu²⁺ is one that has a lower binding affinity for the copper antagonist than Cu²⁺. Preferably, the non-copper metal ion has an association constant for triethylenetetramine that is equal to or less than about 10⁻¹⁹, more preferably less than or equal to about 10⁻¹⁸, still more preferably less than or equal to about 10⁻¹⁵, even more preferably less than or equal to about 10⁻¹², 10⁻¹⁰, or 10⁻⁹, and most preferably less than or equal to about 10⁻⁸, 10⁻⁷ or 10⁻⁵. Preferred metal ions for precomplexing include, for example, calcium (e.g., Ca²⁺), magnesium (e.g., Mg²⁺), chromium (e.g., Cr²⁺ and Cr³⁺), manganese (e.g., Mn²⁺), zinc (e.g., Zn²⁺), and iron (e.g., Fe²⁺ and Fe³⁺). Most preferred metal ions for precomplexing are calcium, zinc, and iron. Other metals include, for example, cobalt (e.g., Co²⁺), nickel (e.g., Ni²⁺), silver (e.g., Ag¹⁺) and selenium (e.g., Se⁴⁺). Non-copper metals are chosen with regard, for example, to their relative binding to the copper antagonist, the dose of the copper antagonist to be administered, and relative to potential toxicity following displacement of the non-copper metal ion. In addition to free copper antagonist compounds and salts thereof, active metabolites, derivatives, and prodrugs of copper antagonists can also be used for precomplexing. Preferred copper antagonists for precomplexing are Cu²⁺ antagonists, particularly Cu²⁺ chelators. Preferred Cu²⁺ antagonists are linear, branched or cyclic polyamines chelators including, for example, tetramines. A preferred tetramine is triethylenetetramine. Examples of precomplexed copper antagonists include precomplexed triethylenetetramines. Precomplexed triethylenetetramines include, for example, triethylenetetramine (or salts thereof, such as triethylenetetramine dihydrocholoride) precomplexed with a metal ion having a binding constant lower than copper. Such compounds may be referred to, for example, as “Ca-Trientine” to refer to triethylenetetramine precomplexed with calcium (e.g., Ca²⁺). Other copper antagonists include D-pencillamine, sar (N-methylglycine), diamsar (1,8-diamino-3,6,10,13,16,19-hexa-azabicyclo[6.6.6]icosane), N-acetylpenicillamine, N,N′-diethyldithiocarbamate, bathocuproinedisulfonic acid, bathocuprinedisulfonate, and thiomolybdates, including mono-, di-, tri- and tetrathiomolybdates. Each may be precomplexed with a metal ion. Precomplexed copper antagonists, for example, a precomplexed triethylenetetramine, may be prepared as the precomplexed compound or a salt thereof. Without intending to be bound to any particular mechanism or mode of action, precomplexing is believed to assist in the preparation, stability, or bioavailability of copper antagonists, including those in to be prepared and administered in aqueous formulations, such as, for example, triethylenetetramine dihydrocholoride. This allows lower dosing as well. Precomplexed copper antagonists may be present in the compositions of the invention in an amount, for example, that is effective to (1) increase copper output in the urine of said subject, (2) decrease body and/or tissue copper levels, (3) decrease copper uptake, for example, in the gastrointestinal tract, (4) decrease SOD, for example, EC-SOD, as measured by mass or activity, (5) decrease homocysteine, (6) decrease oxidative stress and/or (7) increase copper (I).

Also encompassed are metal complexes comprising copper antagonists and non-copper metals (that have lower binding affinities than copper for the copper antagonist) and one or more additional ligands than typically found in complexes of that metal. These additional ligands may serve to block sites of entry into the complex for water, oxygen, hydroxide, or other species that may undesirably complex with the metal ion and can cause degradation of the copper antagonist. For example, copper complexes of triethylenetetramine have been found to form pentacoordinate complexes with a tetracoordinated triethylenetetramine and a chloride ligand when crystallized from a salt solution rather than a tetracoordinate Cu²⁺ triethylenetetramine complex. In this regard, 219 mg of triethylenetetramine*2 HCl were dissolved in 50 ml, and 170 mg of CuCl₂*2H2O were dissolved in 25 ml ethanol (95%). After addition of the CuCl₂ solution to the triethylenetetramine solution, the color changed from light to dark blue and white crystals precipitated. The crystals were dissolved by addition of a solution of 80 mg NaOH in 15 ml H2O. After the solvent was evaporated, the residue was dissolved in ethanol, and two equivalents of ammonium-hexafluorophosphate were added. Blue crystals could be obtained after reduction of the solvent. Crystals were found that were suitable for x-ray structure determination. X-ray crystallography revealed a [Cu(triethylenetetramine)Cl] complex. Other coordinated complexes may be formed from or between copper antagonists, for example, copper chelators (such as Cu2+ chelators, spermadine, spermine, tetracyclam, etc.), particularly those subject to degradative pathways such as those noted above, by providing additional complexing agents (such as anions in solution, for example, I⁻, Br⁻, F⁻, (SO₄)²⁻, (CO₃)²⁻, BF⁴⁻, NO³⁻, ethylene, pyridine, etc.) in solutions of such complexes. This may be particularly desirable for complexes with more accessible metal ions, such as planar complexes or complexes having four or fewer coordinating agents, where one or more additional complexing agents could provide additional shielding to the metal from undesirable ligands that might otherwise access the metal and displace a desired complexing agent.

Trientine active agents may be prepared in a number of ways. Trientine is a strongly basic moiety with multiple nitrogens that can be converted into a large number of suitable associated acid addition salts using an acid, for example, by reaction of stoichiometrically equivalent amounts of trientine and of the acid in an inert solvent such as ethanol or water and subsequent evaporation if the dosage form is best formulated from a dry salt. Possible acids for this reaction are in particular those that yield physiologically acceptable salts.

Nitrogen-containing copper antagonists, for example, trientine active agents such as, for example, trientine, that can be delivered as a salt(s) (such as acid addition salts, e.g., trientine dihydrochloride) act as copper-chelating agents or antagonists, which aids the elimination of copper from the body by forming a stable soluble complex that is readily excreted by the kidney. Thus inorganic acids can be used, e.g., sulfuric acid, nitric acid, hydrohalic acids such as hydrochloric acid or hydrobromic acid, phosphoric acids such as orthophosphoric acid, sulfamic acid. This is not an exhaustive list. Other organic acids can be used to prepare suitable salt forms, in particular aliphatic, alicyclic, araliphatic, aromatic or heterocyclic mono-or polybasic carboxylic, sulfonic or sulfuric acids, (e.g., formic acid, acetic acid, propionic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methanesulfonic acid, ethanesulfonic acid, ethanedisulfonic acid, 2-hydroxyethanesulfonic acid, benzenesulfonic acid, p-toluenesulfonic acid, naphthalenemono-and-disulfonic acids, and laurylsulfuric acid). Hydrochloric acid, fumaric acid, maleic acid and succinic acid salts are preferred, and succinic acid salts are most preferred. Those in the art will be able to prepare other suitable salt forms.

Nitrogen-containing copper antagonists, for example, trientine active agents such as, for example, trientine, can also be in the form of quartemary ammonium salts in which the nitrogen atom carries a suitable organic group such as an alkyl, alkenyl, alkynyl or aralkyl moiety. In one embodiment such nitrogen-containing copper antagonists are in the form of a compound or buffered in solution and/or suspension to a near neutral pH much lower than the pH 14 of a solution of trientine itself.

Other trientine active agents include derivative trientines, for example, trientine in combination with picolinic acid (2-pyridinecarboxylic acid). These derivatives include, for example, trientine picolinate and salts of trientine picolinate, for example, trientine picolinate HCl. They also include, for example, trientine di-picolinate and salts of trientine di-picolinate, for example, trientine di-picolinate HCl. Picolinic acid moieties may be attached to trientine, for example one or more of the CH₂ moieties, using chemical techniques known in the art. Those in the art will be able to prepare other suitable derivatives, for example, trientine-PEG derivatives, which may be useful for particular dosage forms including oral dosage forms having increased bioavailability.

Compounds suitable as copper antagonists include cyclic and acyclic compounds according to Formula I:

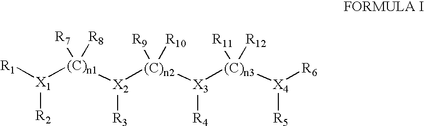

wherein X₁, X₂, X₃ and X₄ are independently selected from the group consisting of N, S and O; R₁, R₂, R₃, R₄, R₅ and R₆ are independently selected from the group consisting of H, C₁ to C₁₀ straight chain or branched alkyl, C3 to C10 cycloalkyl, C1 to C6 alkyl C3 to C10 cycloalkyl, anyl, anyl substituted with 1 to 5 substituents, heteroaryl, fused aryl, C1 to C6 alkyl aryl, C1 to C6 alkyl aryl substituted with 1 to 5 substituents, C1 to C5 alkyl heteroaryl, C1 to C6 alkyl fused aryl, —CH₂COOH, —CH₂SO₃H, —CH₂PO(OH)₂, and —CH₂P(CH₃)O(OH); n1, n2 and are independently 2 or 3 and each of R₇, R₈, R₉, R₁₀, R₁₁ and R₁₂ is independently selected and is selected from the group consisting of H, C1 to C10 straight chain or branched alkyl, C3 to C10 cycloalkyl, C1 to C6 alkyl, C3 to C10 cycloalkyl, aryl, aryl substituted with 1 to 5 substituents, heteroaryl fused aryl, C1 to C6 alkyl aryl, C1 to C6 alkyl aryl substituted with 1 to 5 substituents, C1 to C5 alkyl heteroaryl, C1 to C6 fused aryl, provided that when X₁ is S or O, then R₂ is absent; when X₂ is S or O, then R₃ is absent, when X₃ is S or O, then R₄ is absent and when X₄ is S or O, then R₅ is absent.

Optionally, one or more of R₁, R₂, R₃, R₄, R₅ and R₆ may be functionalized for attachment to groups which include, but are not limited to peptides, proteins, polyethylene glycols (PEGs) and other suitable chemical entities in order to modify the overall pharmacokinetics, deliverability and/or half lives of the constructs. Examples of such functionalization include, but are not limited to, C1 to C10 alkyl-CO-peptide, C1 to C10 alkyl-CO-protein, C1 to C10 alkyl-CO-PEG, C1 to C10 alkyl-NH-peptide, C1 to C10 alkyl-NH-protein, C1 to C10 alkyl-NH-CO-PEG, C1 to C10 alkyl-S-peptide, and C1 to C10 alkyl-S-protein.

In addition, optionally are one or more of R₇, R₈, R₉, R₁₀, R₁₁ and R₁₂ may be functionalized for attachment to groups which include, but are not limited to, peptides, proteins, polyethylene glycols (PEGs) and other suitable chemical entities in order to modify the overall pharmacokinetics, deliverability and/or half lives of the constructs. Examples of such functionalization include, but are not limited to, C1 to C10 alkyl-CO-peptide, C1 to C10 alkyl-CO-protein, C1 to C10 alkyl-CO-PEG, C1 to C10 alkyl-NH-peptide, C1 to C10 alkyl-NH-protein, C1 to C10 alkyl-NH—CO-PEG, C1 to C10 alkyl-S-peptide and C1 to C10 alkyl-S-protein.

One group of suitable compounds of Formula I include those wherein R₁, R₂, R₃, R₄, R₅ and R₆ are independently selected from H, C1 to C6 alkyl, —CH₂COOH, —CH₂SO₃H, —CH₂PO(OH)₂ and —CH₂P(CH₃)O(OH); and each R₇, R₈, R₉, R₁₀, R₁₁ and R₁₂ is independently selected from H and C1 to C6 alkyl. In one aspect, suitable compounds include those wherein at least one of R₁ and R₂ and at least one of R₅ and R₆ is H or C1 to C6 alkyl. According to this aspect, suitably R₃ and R₄ are selected from H or C1 to C6 alkyl; more particularly, R₁, R₂, R₅, and R₆ are selected from H or C1 to C6 alkyl. One sub-group of suitable compounds include those wherein X₂ and X₃ are N and n1, n2 and n3 are 2, or n1 and n3 are 2 and n2 is 3. In this sub-group, R₁, R₆, R₇, R₈, R₉, R₁₀, R₁₁ and R₁₂ are independently selected from H and C1 to C3 alkyl. According to another sub-group of suitable compounds, all of X₁, X₂, X₃, and X₄ are suitably N or, alternatively, one of X₁ and X₄ is S and X₂ and X₃ are N or S.

Tetra-heteroatom acyclic compounds within Formula I are provided where X₁, X₂, X₃, and X₄ are independently chosen from the atoms N, S or O, such that,

(a) for a four-nitrogen series, i.e., when X₁, X₂, X₃, and X₄ are N then: R₁, R₂, R₃, R₄, R₅, and R₆ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and, R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R₁, R₂, R₃, R₄, R₅, or R₆ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein. Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH-CO-PEG, C1-C10 alkyl-S-peptide, and C1-C10 alkyl-S-protein.

(b) for a first three-nitrogen series, i.e., when X₁, X₂, X₃, are N and X₄ is S or O then: R₆ does not exist; R₁, R₂, R₃, R₄ and R₅ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and, R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R₁, R₂, R₃, R₄, or R₅ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein. Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(c) for a second three-nitrogen series, i.e., when X₁, X₂, and X₄ are N and X₃ is O or S then: R₄ does not exist and R₁, R₂, R₃, R₅, and R₆ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and, R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R₁, R₂, R₃, R₅, or R₆ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein. Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(d) for a first two-nitrogen series, i.e., when X₂ and X₃ are N and X₁ and X₄ are O or S then: R₁ and R₆ do not exist; R₂, R₃, R₄, and R₅ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R₂, R₃, R₄, or R₅ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein. Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(e) for a second two-nitrogen series, i.e., when X₁ and X₃ are N and X₂ and X₄ are O or S then: R₃ and R₆ do not exist; R₁, R₂, R₄, and R₅ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and R₇, R₈, R₉, R₁₀, R₁₁, and R₁₂ are independently chosen from H, CH₃, C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R₁, R₂, R₄, or R₅ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(f) for a third two-nitrogen series, i.e., when X₁, and X₂ are N and X₃ and X₄ are O or S then: R₄ and R₆ do not exist; R₁, R₂, R₃, and R₅ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R₁, R₂, R₃, or R₅ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(g) for a fourth two-nitrogen series, i.e., when X₁ and X₄ are N and X₂ and X₃ are O or S then: R₃ and R₄ do not exist; R₁, R₂, R₅ and R₆ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R₁, R₂, R₅, or R₆ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Second, for a tetra-heteroatom series of cyclic analogues, one of R₁ and R₂ and one of R₅ and R₆ are joined together to form the bridging group (CR₁₃R₁₄)ₙ₄, and X₁, X₂, X₃, and X₄ are independently chosen from the atoms N, S or O such that,

(a) for a four-nitrogen series, i.e., when X₁, X₂, X₃, and X₄ are N then: R₂, R₃, R₄, and R₅ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, n3, and n4 are independently chosen to be 2 or 3; and R₇, R₈, R₉, R₁₀, R₁₁, R₁₂, R₁₃ and R₁₄ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R₂, R₃, R₄, or R₅ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein. Furthermore one or several of R₇, R₈, R₉, R₁₀, R₁₁, R₁₂, R₁₃ or R₁₄ may be functionalized for attachment, for example, to peptides, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH-CO-PEG, C1-C10 alkyl-S-peptide, and C1-C10 alkyl-S-protein.

(b) for a three-nitrogen series, i.e., when X₁, X₂, X₃, are N and X₄ is S or O then: R₅ does not exist; R₂, R₃, and R₄ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, n3, and n4 are independently chosen to be 2 or 3; and R₇, R₈, R₉, R₁₀, R₁₁, R₁₂, R₁₃ and R₁₄ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R₂, R₃ or R4 may be functionalized for attachment, for example, to peptides, proteins, polyethylene glycols and other such chemical entities in order to modify the overall pharmacokinetics, deliverability and/or half-lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R₇, R₈, R₉, R₁₀, R₁₁, R₁₂, R₁₃ or R₁₄ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(c) for a first two-nitrogen series, i.e., when X₂ and X₃ are N and X₁ and X₄ are O or S then: R₂ and R₅ do not exist; R₃ and R₄ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, n3, and n4 are independently chosen to be 2 or 3; and R₇, R₈, R₉, R₁₀, R₁₁, R₁₂, R₁₃ and R₁₄ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or both of R₃, or R₄ may be functionalized for attachment, for example, to peptides, proteins, polyethylene glycols and other such chemical entities in order to modify the overall pharmacokinetics, deliverability and/or half-lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R₇, R₈, R₉, R₁₀, R₁₁, R₁₂, R₁₃ or R₁₄ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(d) for a second two-nitrogen series, i.e., when X₁ and X₃ are N and X₂ and X₄ are O or S then: R₃ and R₅ do not exist; R₂ and R₄ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, n3, and n4 are independently chosen to be 2 or 3; and R₇, R₈, R₉, R₁₀, R₁₁, R₁₂, R₁₃ and R₁₄ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or both of R₂, or R₄ may be functionalized for attachment, for example, to peptides, proteins, polyethylene glycols and other such chemical entities in order to modify the overall pharmacokinetics, deliverability and/or half-lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R₇, R₈, R₉, R₁₀, R₁₁, R₁₂, R₁₃ or R₁₄ may be functionalized for attachment, for example, to peptides, proteins, polyethylene glycols and other such chemical entities in order to modify the overall pharmacokinetics, deliverability and/or half lives of the constructs. Examples of such finctionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(e) for a one-nitrogen series, i.e., when X₁ is N and X₂, X₃ and X₄ are O or S then: R₃, R₄ and R₅ do not exist; R₂ is independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, n3, and n4 are independently chosen to be 2 or 3; and R₇, R₈, R₉, R₁₀, R₁₁, R₁₂, R₁₃ and R₁₄ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, R₂ may be functionalized for attachment, for example, to peptides, proteins, polyethylene glycols and other such chemical entities in order to modify the overall pharmacokinetics, deliverability and/or half lives of the constructs. Examples of such fimctionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R₇, R₈, R₉, R₁₀, R₁₁, R₁₂, R₁₃ or R₁₄ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Suitable copper antagonist compounds of Formula I include, for example:

SH—CH₂—CH₂—NH—CH₂—CH₂—NH—CH₂—CH₂—NH₂,

SH—CH₂—CH₂—S—CH₂—CH₂—NH—CH₂—CH₂—NH₂,

NH₂—CH₂—CH₂—NH—CH₂—CH₂—S—CH₂—CH₂—SH,

NH₂—CH₂—CH₂—S—CH₂—CH₂—S—CH₂—CH₂—SH,

SH—CH₂—CH₂—S—CH₂—CH₂—S—CH₂—CH₂—SH,

NH₂—CH₂—CH₂—NH—CH₂—CH₂—CH₂—NH—CH₂—CH₂—NH₂,

SH—CH₂—CH₂—NH—CH₂—CH₂—CH₂—NH—CH₂—CH₂—NH₂,

SH—CH₂—CH₂—S—CH₂—CH₂—CH₂—NH—CH₂—CH₂—NH₂,

NH₂—CH₂—CH₂—NH—CH₂—CH₂—CH₂—S—CH₂—CH₂—SH,

NH₂—CH₂—CH₂—S—CH₂—CH₂—CH₂—S—CH₂—CH₂—SH, and

SH—CH₂—CH₂—S—CH₂—CH₂—CH₂—S—CH₂—CH₂—SH.

Suitable compounds of Formula I include, for example, one or more of triethylenetetramine, salts of triethylenetetramine, prodrugs of triethylenetetramine and salts of such prodrugs; analogs of triethylenetetramine and salts and prodrugs of such analogs; and/or active metabolites of triethylenetetramine and salts and prodrugs of such metabolites, including but not limited to N-acetyl triethylenetetramine and salts and prodrugs of N-acetyl triethylenetetramine.

Triethylenetetramine is a strongly basic moiety with multiple nitrogens that can be converted into a large number of suitable associated acid addition salts using an acid, for example, by reaction of triethylenetetramine and of the acid, for example, stoichiometrically equivalent amounts, in a solvent, for example, an inert solvent such as, for example, ethanol or water and subsequent evaporation if the dosage form is best formulated from a dry salt. Possible acids for this reaction are in particular those that yield physiologically acceptable salts. Nitrogen-containing copper chelator(s) or binding compound(s), for example, trientine active agents such as, for example, triethylenetetramine, that can be delivered as a salt(s) (such as acid addition salts, e.g., triethylenetetramine dihydrochloride or triethylenetetramine disuccinate or other acceptable hydrochloride or succinate salts), act as copper-chelating or binding agents, which aids the elimination of copper from the body by forming a stable soluble complex that is readily excreted by the kidney. Thus, inorganic acids can be used, e.g., sulfuric acid, nitric acid, hydrohalic acids such as hydrochloric acid or hydrobromic acid, phosphoric acids such as orthophosphoric acid, and sulfamic acid. This is not an exhaustive list. Other organic acids can be used to prepare suitable salt forms, in particular aliphatic, alicyclic, araliphatic, aromatic or heterocyclic mono-or polybasic carboxylic, sulfonic or sulfuric acids, (e.g., formic acid, acetic acid, propionic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methane-or ethanesulfonic acid, ethanedisulfonic acid, 2-hydroxyethanesulfonic acid, benzenesulfonic acid, p-toluenesulfonic acid, naphthalenemono-and-disulfonic acids, and laurylsulfuric acid). Organic acids include both aliphatic and aromatic carboxylic acids and include, for example, aliphatic monocarboxylic acids, aliphatic dicarboxylic acids, aliphatic tricarboxylic acids, aromatic monocarboxylic acids, aromatic dicarboxylic acids, aromatic tricarboxylic acids and other organic acids known to those of skill in the art. Aliphatic carboxylic acids may be saturated or unsaturated. Suitable aliphatic carboxylic acids include those having from 2 to about 10 carbon atoms. Aliphatic monocarboxylic acids include saturated aliphatic monocarboxylic acids and unsaturated aliphatic monocarboxylic acids. Examples of saturated monocarboxylic acids include acetic acid, propronic acid, butyric acid, valeric acid, caproic acid, enanthic acid, caprylic acid, pelargonic acid, and caprynic acid. Examples of unsaturated aliphatic monocarboxylic acids include acrylic acid, propiolic acid, methacrylic acid, crotonic acid and isocrotonic acid. Aliphatic dicarboxylic acids include saturated aliphatic dicarboxylic acids and unsaturated aliphatic dicarboxylic acids. Examples of saturated aliphatic dicarboxylic acids include oxalic acid, malonic acid, succinic acid, glutaric acid, adipic acid, pimelic acid, suberic acid, azelaic acid, and sebacic acid. Examples of unsaturated aliphatic dicarboxylic acids include maleic acid, fumaric acid, citraconic acid, mesaconic acid, itaconic acid and the like. Aliphatic tricarboxylic acids includes saturated aliphatic tricarboxylic acids and unsaturated tricarboxylic acids. Examples of saturated tricarboxylic acids include tricarballylic acid, 1,2,3-butanetricarboxylic acid and ihe like. Suitable aliphatic dicarboxylic acids include those of the formula: HOOC-Q₁-COOH, wherein Q₁ is alkylene of 1 to about 8 carbon atoms or alkenylene of 2 to about 8 atoms, and includes both straight chain and branched chain alkylene and alkenylene groups. Examples of aromatic dicarboxylic acids include phthalic acid, isophthalic acid, terephthalic acid and the like. Examples of aromatic tricarboxylic acids include trimesic acid, hemimellitic acid and trimellitic acid.

Those in the art will be able to prepare other suitable salt forms. Nitrogen-containing copper chelator(s) or binding compound(s), for example, trientine active agents such as, for example, triethylenetetramine, can also be in the form of quarternary ammonium salts in which the nitrogen atom carries a suitable organic group such as an alkyl, alkenyl, alkynyl or aralkyl moiety. In one embodiment, such nitrogen-containing copper chelator(s) are in the form of a compound or buffered in solution and/or suspension nearer to a neutral pH, lower than the pH 14 of a solution of triethylenetetramine itself.

Other trientine active agents include derivative trientine active agents, for example, triethylenetetramine in combination with picolinic acid (2-pyridinecarboxylic acid). These derivatives include, for example, triethylenetetramine picolinate and salts of triethylenetetramine picolinate, for example, triethylenetetramine picolinate HCl. These also include, for example, triethylenetetramine di-picolinate and salts of triethylenetetramine di-picolinate, for example, triethylenetetramine di-picolinate HCl. Picolinic acid moieties may be attached to triethylenetetramine, for example, one or more of the CH₂ moieties, using chemical techniques known in the art. Those in the art will be able to prepare other suitable derivatives, for example, triethylenetetramine-PEG derivatives, which may be useful for particular dosage forms including oral dosage forms having increased bioavailability.

According to one aspect, compounds suitable as copper antagonists include compounds of Formula I(a):

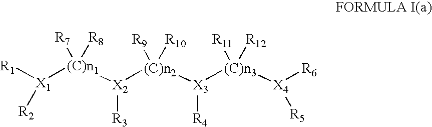

wherein X₁, X₂, X₃ and X₄ are N or one of X₁, X₂, X₃ and X₄ is O or S and the remainder are N; n₁, n₂, and n₃ are 2 or 3; R₁, R₂, R₃, R₄, R₅ and R₆ are H or absent; and R₇, R₈, R₉, R₁₀, R₁₁, and R₁₂, are independently selected from the group consisting of H, CH₃ and CH₂CH₃ and wherein; if X₁ is S or O, then R₂ is absent; if X₂ is S or O, the R₃ is absent; if X₃ is S or O, then R₄ is absent; and if X₄ is S or O, then R₆ is absent.

Additional compounds suitable as copper antagonists include cyclic and acyclic compounds according to Formula II:

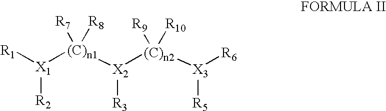

wherein X₁, X₂ and X₃ are independently selected from the group consisting of N, S and O; R₁, R₂, R₃, R₅ and R₆ are independently selected from the group consisting of H, C₁ to C₁₀ straight chain or branched alkyl, C3 to C10 cycloalkyl, C1 to C6 alkyl C3 to C10 cycloalkyl, anyl, anyl substituted with 1 to 5 substituents, heteroaryl, fused aryl, C1 to C6 alkyl aryl, C1 to C6 alkyl aryl substituted with 1 to 5 substituents, C1 to C5 alkyl heteroaryl, C1 to C6 alkyl fused aryl, —CH₂COOH, —CH₂SO₃H, —CH₂PO(OH)₂, and —CH₂P(CH₃)O(OH); n1 and n2 are independently 2 or 3 and each of R₇, R₈, R₉ and R₁₀, is independently selected and is selected from the group consisting of H, C1 to C10 straight chain or branched alkyl, C3 to C10 cycloalkyl, C1 to C6 alkyl, C3 to C10 cycloalkyl, aryl, aryl substituted with 1 to 5 substituents, heteroaryl fused aryl, C1 to C6 alkyl aryl, C1 to C6 alkyl aryl substituted with 1 to 5 substituents, C1 to C5 alkyl heteroaryl, C1 to C6 fused aryl, provided that when X₁ is S or O, then R₂ is absent; when X₂ is S or O, then R₃ is absent, and when X₃ is S or O, then R₅ is absent.

Optionally, one or more of R₁, R₂, R₃, R₅ and R₆ may be functionalized for attachment to groups which include, but are not limited to peptides, proteins, polyethylene glycols (PEGs) and other suitable chemical entities in order to modify the overall pharmacokinetics, deliverability and/or half lives of the constructs. Examples of such functionalization include, but are not limited to, C1 to C10 alkyl-CO-peptide, C1 to C10 alkyl-CO-protein, C1 to C10 alkyl-CO-PEG, C1 to C10 alkyl-NH-peptide, C1 to C10 alkyl-NH-protein, C1 to C10 alkyl-NH—CO-PEG, C1 to C10 alkyl-S-peptide, and C1 to C10 alkyl-S-protein.

In addition, optionally are one or more of R₇, R₈, R₉, and R₁₀ may be functionalized for attachment to groups which include, but are not limited to, peptides, proteins, polyethylene glycols (PEGs) and other suitable chemical entities in order to modify the overall pharmacokinetics, deliverability and/or half lives of the constructs. Examples of such functionalization include, but are not limited to, C1 to C10 alkyl-CO-peptide, C1 to C10 alkyl-CO-protein, C1 to C10 alkyl-CO-PEG, C1 to C10 alkyl-NH-peptide, C1 to C10 alkyl-NH-protein, C1 to C10 alkyl-NH—CO-PEG, C1 to C10 alkyl-S-peptide and C1 to C10 alkyl-S-protein.

One group of suitable compounds of Formula I include those wherein R₁, R₂, R₃, R₅ and R₆ are independently selected from H, C1 to C6 alkyl, —CH₂COOH, —CH₂SO₃H, —CH₂PO(OH)₂ and —CH₂P(CH₃)O(OH); and each R₇, R₈, R₉ and R₁₀ is independently selected from H and C1 to C6 alkyl. In one aspect, suitable compounds include those wherein at least one of R₁ and R₂ and at least one of R₅ and R₆ is H or C1 to C6 alkyl. According to this aspect, suitably R₃ is selected from H or C1 to C6 alkyl; more particularly, R₁, R₂, R₅, and R₆ are selected from H or C1 to C6 alkyl. One sub-group of suitable compounds include those wherein R₆, R₇, R₈, R₉ and R₁₀, are independently selected from H and C1 to C3 alkyl. According to another sub-group of suitable compounds, all of X₁, X₂ and X₃ are suitably N or, alternatively, one of X₁ and X₃ is S and X₂ are N or S.

Tri-heteroatom compounds within Formula II are provided where X₁, X₂, and X₃ are independently chosen from the atoms N, S or O such that,

(a) for a three-nitrogen series, when X₁, X₂, and X₃ are N then: R₁, R₂, R₃, R₅, and R₆ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, and n2 are independently chosen to be 2 or 3; and R₇, R₈, R₉, and R₁₀ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R₁, R₂, R₃, R₅ or R₆ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R₇, R₈, R₉, or R₁₀ may be functionalized for attachment, for example, to peptides, proteins, polyethylene glycols and other such chemical entities in order to modify the overall pharmacokinetics, deliverability and/or half-lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(b) for a first two-nitrogen series, when X₁ and X₂ are N and X₃ is S or O then: R₃ does not exist; R₁, R₂, R₅, and R₆ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, and n2 are independently chosen to be 2 or 3; and R₇, R₈, R₉, and R₁₀ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R₁, R₂, R₅ or R₆ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R₇, R₈, R₉, or R₁₀ may be fimctionalized for attachment, for example, to peptides, proteins, polyethylene glycols and other such chemical entities in order to modify the overall pharmacokinetics, deliverability and/or half-lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(c) for a second, two-nitrogen series, when X₁ and X₂ are N and X₃ is O or S then: R₅ does not exist; R₁, R₂, R₃, and R₆ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1 and n2 are independently chosen to be 2 or 3; and R₇, R₈, R₉, and R₁₀ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R₁, R₂, R₅, or R₆ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R₇, R₈, R₉, or R₁₀ may be functionalized for attachment, for example, to peptides, proteins, polyethylene glycols and other such chemical entities in order to modify the overall pharmacokinetics, deliverability and/or half-lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

A series of tri-heteroatom cyclic analogues according to the above Formula II are provided in which R₁ and R₆ are joined together to form the bridging group (CR₁₁R₁₂)ₙ₃, and X₁, X₂ and X₃ are independently chosen from the atoms N, S or O such that:

(a) for a three-nitrogen series, when X₁, X₂, and X₃ are N then: R₂, R₃, and R₅ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or several of R₂, R₃, or R₅ may be finctionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(b) for a two-nitrogen series, when X₁ and X₂ are N and X₃ is S or O then: R₅ does not exist; R₂, and R₃ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, one or both of R₂ or R₃ may be functionalized for attachment, for example, to peptides, proteins, polyethylene glycols and other such chemical entities in order to modify the overall pharmacokinetics, deliverability and/or half-lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

(c) for a one-nitrogen series, when X₁ is N and X₂ and X₃ are O or S then: R₃ and R₅ do not exist; R₂ is independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH); n1, n2, and n3 are independently chosen to be 2 or 3; and R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl. In addition, R₂ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein. Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmacokinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Copper antagonists useful in the invention also include copper chelators that have been pre-complexed with a non-copper metal ion prior to administration for therapy. Metal ions used for pre-complexing have a lower association constant for the copper antagonist than that of copper. For example, a metal ion for pre-complexing a copper antagonist that chelates Cu²⁺ is one that has a lower binding affinity for the copper antagonist than Cu²⁺. Preferred metal ions for precomplexing include calcium (e.g., Ca²⁺), magnesium (e.g., Mg²⁺), chromium (e.g., Cr²⁺ and Cr³⁺), manganese (e.g., Mn²⁺), zinc (e.g., Zn²⁺), selenium (e.g., Se⁴⁺), and iron (e.g., Fe²⁺ and Fe³⁺). Most preferred metal ions for precomplexing are calcium, zinc, and iron. Other metals include, for example, cobalt (e.g., Co²⁺), nickel (e.g., Ni²⁺), silver (e.g., Ag¹⁺), and bismuth (e.g., Bi³⁺). Metals are chosen with regard, for example, to their relative binding to the copper antagonist, and relative to toxicity and the dose of the copper antagonist to be administered.

Also encompassed are metal complexes comprising copper antagonists and non-copper metals (that have lower binding affinities than copper for the copper antagonist) and one or more additional ligands than typically found in complexes of that metal. These additional ligands may serve to block sites of entry into the complex for water, oxygen, hydroxide, or other species that may undesirably complex with the metal ion and can cause degradation of the copper antagonist. For example, copper complexes of triethylenetetramine have been found to form pentacoordinate complexes with a tetracoordinated triethylenetetramine and a chloride ligand when crystallized from a salt solution rather than a tetracoordinate Cu²⁺ triethylenetetramine complex. In this regard, 219 mg of triethylenetetramine 2 HCl were dissolved in 50 ml, and 170 mg of CuCl₂.2H2O were dissolved in 25 ml ethanol (95%). After addition of the CuCl₂ solution to the triethylenetetramine solution, the color changed from light to dark blue and white crystals precipitated. The crystals were dissolved by addition of a solution of 80 mg NaOH in 15 ml H2O. After the solvent was evaporated, the residue was dissolved in ethanol, and two equivalents of ammonium-hexafluorophosphate were added. Blue crystals could be obtained after reduction of the solvent. Crystals were found that were suitable for x-ray structure determination. X-ray crystallography revealed a [Cu(triethylenetetramine)Cl] complex. Other coordinated complexes may be formed from or between copper antagonists, for example, copper chelators (such as Cu2+ chelators, spermidine, spermine, tetracyclam, etc.), particularly those subject to degradative pathways such as those noted above, by providing additional complexing agents (such as anions in solution, for example, I⁻, Br⁻, F⁻, (SO₄)²⁻, (CO₃)²⁻, BF⁴⁻, NO³⁻, ethylene, pyridine, etc.) in solutions of such complexes. This may be particularly desirable for complexes with more accessible metal ions, such as planar complexes or complexes having four or fewer coordinating agents, where one or more additional complexing agents could provide additional shielding to the metal from undesirable ligands that might otherwise access the metal and displace a desired complexing agent.

The compounds for use according to the present invention, including trientine active agents, may be made using any of a variety of chemical synthesis, isolation, and purification methods known in the art. For example, Published United States Patent Application No. 2006/0041170 describes the synthesis of certain triethylenetetramine salts. Exemplary synthetic routes are described below.

General synthetic chemistry protocols are somewhat different for these classes of molecules due to their propensity to chelate with metallic cations, including copper. Glassware should be cleaned and silanized prior to use. Plasticware should be chosen specifically to have minimal presence of metal ions. Metal implements such as spatulas should be excluded from any chemistry protocol involving chelators. Water used should be purified by sequential carbon filtering, ion exchange and reverse osmosis to the highest level of purity possible, not by distillation. All organic solvents used should be rigorously purified to exclude any possible traces of metal ion contamination.

Care must also be take with purification of such derivatives due to their propensity to chelate with a variety of cations, including copper, which may be present in trace amounts in water, on the surface of glass or plastic vessels. Once again, glassware should be cleaned and silanized prior to use. Plasticware should be chosen specifically to have minimal presence of metal ions. Metal implements such as spatulas should be avoided, and water used should be purified by sequential carbon filtering, ion exchange and reverse osmosis to the highest level of purity possible, and not by distillation. All organic solvents used should be rigorously purified to exclude any possible traces of metal ion contamination. Ion exchange chromatography followed by lyophilization is typically the best way to obtain pure solid materials of these classes of molecules. Ion exchange resins should be washed clean of any possible metal contamination.

Many of the synthetic routes allow for control of the particular R groups introduced. For synthetic methods incorporating amino acids, synthetic amino acids can be used to incorporate a variety of substituent R groups. The dichloroethane synthetic schemes also allow for the incorporation of a wide variety of R groups by using dichlorinated ethane derivatives. It will be appreciated that many of these synthetic schemes can lead to isomeric forms of the compounds; such isomers can be separated using techniques known in the art.

Documents describing aspects of these synthetic schemes include the following: (1) A W von Hoffman, Berichte 23, 3711 (1890); (2) The Polymerization Of Ethylenimine, Giffin D. Jones, Arne Langsjoen, Sister Mary Marguerite Christine Neumann, Jack Zomlefer, J. Org. Chem., 1944; 9(2); 125-147; (3) The peptide way to macrocyclic bifimctional chelating agents: synthesis of 2-(p-nitrobenzyl)-1,4,7,10-tetraazacyclododecane-N,N′,N″,N′″-tetraacetic acid and study of its yttrium(III) complex, Min K. Moi et al., J. Am. Chem. Soc.,1988; 110(18); 6266-6267; (4) Synthesis of a kinetically stable ⁹⁰Y labelled macrocycle-antibody conjugate, Jonathan P L Cox, et al., J. Chem. Soc. Chem. Comm., 797 (1989); (5) Specific and stable labeling of antibodies with technetium-99m with a diamide dithiolate chelating agent, Fritzberg A R, Abrams P G, Beaumier P L, Kasina S, Morgan A C, Rao T N, Reno J M, Sanderson J A, Srinivasan A, Wilbur D S, et al., Proc. Natl. Acad. Sc.i U.S.A. 85(11):4025-4029 (1988 June); (6) Towards tumour imaging with ¹¹¹In labelled macrocycle-antibody conjugates, Andrew S Craig et al., J. Chem. Soc. Chem. Comm., 794 (1989); (7) Synthesis of C- and N-functionalised derivatives of NOTA, DOTA, and DTPA: bifunctional complexing agents for the derivitisation of antibodies, Jonathan P L Cox et al., J. Chem. Soc. Perkin. I, 2567 (1990); (8) Macrocyclic chelators as anticancer agents in radioimmunotherapy, N R A Beeley and P R J Ansell, Current Opinions in Therapeutic Patents, 2:1539-1553 (1992); and (9) Synthesis of new macrocyclic amino-phosphinic acid complexing agents and their C— and P-functionalised derivatives for protein linkage, Christopher J Broan et al., Synthesis, 63 (1992).

Acyclic and cyclic compounds of the invention and exemplary synthetic methods and existing syntheses from the art include the following:

For tetra-heteroatom acyclic examples of Formula I:

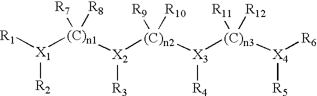

X₁, X₂, X₃, and X₄ are independently chosen from the atoms N, S or O such that: 4N series:
when X₁, X₂, X₃, and X₄ are N then:

R₁, R₂, R₃, R₄, R₅, and R₆ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH);

n1, n2, and n3 are independently chosen to be 2 or 3, and each repeat of any of n1, n2, and n3 may be the same as or different than any other repeat; and

R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R₁, R₂, R₃, R₄, R₅, or R₆ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Also provided are embodiments wherein one, two, three or four of R₁ through R₁₂ are other than hydrogen.

In some embodiments, the compounds of Formulae I, I(a) or II are selective for a particular oxidation state of copper. For example, the compounds may be selected so that they preferentially bind oxidized copper, or copper (II). Copper selectivity can be assayed using methods known in the art. Competition assays can be done using isotopes of copper (I) and copper (II) to determine the ability of the compounds to selectively bind one form of copper.

In some embodiments, the compounds of Formulae I, I(a) or II may be chosen to avoid excessive lipophilicity, for example by avoiding large or numerous alkyl substituents. Excessive lipophilicity can cause the compounds to bind to and/or pass through cellular membranes, thereby decreasing the amount of compound available for chelating copper, particularly for extracellular copper, which may be predominantly in the oxidized form of copper (II).

### Synthesis of Examples of the Open Chain 4N Series of Formula I

Triethylenetetramine itself has been synthesized by reaction of 2 equivalents of ethylene diamine with 1,2-dichloro ethane to give triethylenetetramine directly. Modification of this procedure by using starting materials with appropriate Rₐ and R_{b} groups (where Rₐ, R_{b}=R₇, R₈ or R₁₁, R₁₂) would lead to symmetrically substituted open chain 4N examples as shown below:

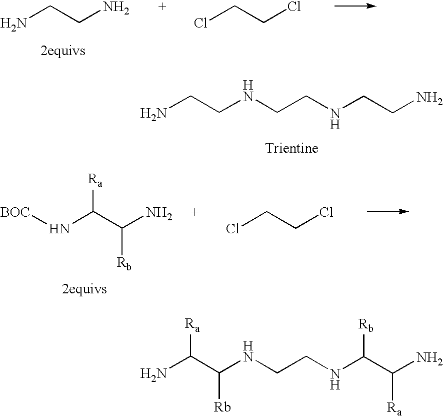

The judicious use of protecting group chemistry such as the widely used BOC (t-butyloxycarbonyl) group allows the chemistry to be directed specifically towards the substitution pattern shown. Other approaches such as via the chemistry of ethyleneimine may also lead to a subset of the tetra-aza series. In order to obtain the un-symmetrically substituted derivatives a variant of some chemistry described by Meares et al. should be used. Standard peptide synthesis using the Rink resin along with FMOC protected natural and un-natural amino acids which can be conveniently cleaved at the penultimate step of the synthesis generates a tri-peptide C-terminal amide. This is reduced using Diborane in THF to give the open chain tetra-aza compounds as shown below:

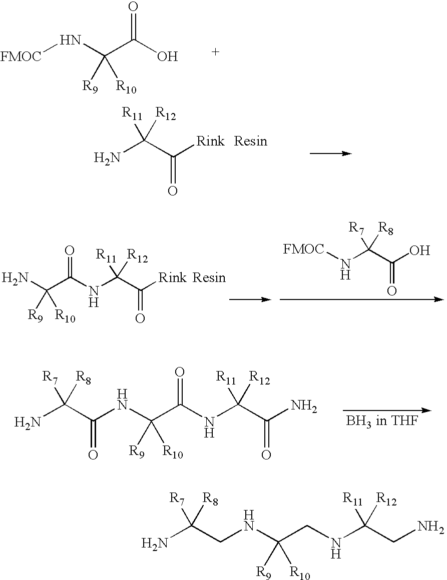

The incorporation of R₁, R₂, R₅ and R₆ can be accomplished with this chemistry by standard procedures.

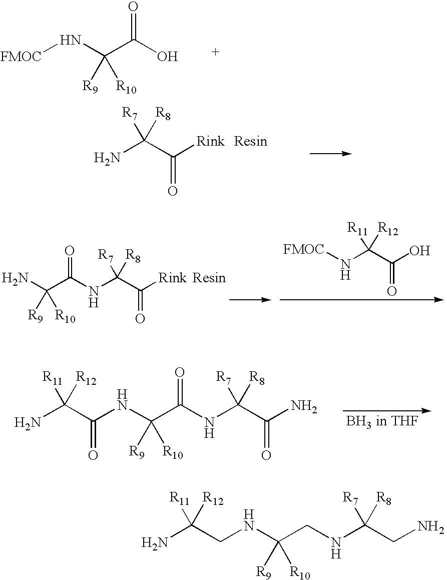

The reverse Rink approach, shown above, also leads to this class of tetra-aza derivatives and may be useful in cases where peptide coupling of a sterically hindered amino acid requires multiple coupling attempts in order to achieve success in the initial Rink approach.

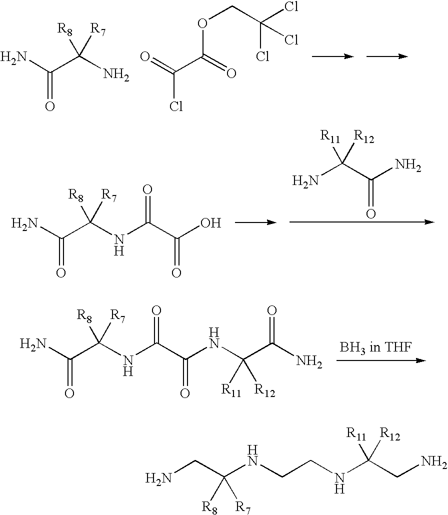

The oxalamide approach, shown above, also can lead to successful syntheses of this class of compounds, although the central substituents are always going to be hydrogen or its isotopes with this kind of chemistry. This particular variant makes use of the trichloroethyl ester group to protect one of the carboxylic acid fumctions of oxalic acid but other protecting groups are also envisaged. Reaction of an amino acid amide derived from a natural or unnatural amino acid with a differentially protected oxalyl mono chloride gives the mono-oxalamide shown which can be reacted under standard peptide coupling condition to give the un-symmetrical bis-oxalamide which can then be reduced with diborane to give the desired tetra-aza derivative.

3NX series 1:

when X₁, X₂, X₃, are N and X₄ is S or O then:

R₆ does not exist

R₁, R₂, R₃, R₄ and R₅ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH);

n1, n2, and n3 are independently chosen to be 2 or 3, and each repeat of any of n1, n2, and n3 may be the same as or different than any other repeat; and

R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R₁, R₂, R₃, R₄, or R₅ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

### Synthesis of Examples of the Open Chain 3NX Series 1 of Formula I

Variations of the syntheses used for the 4N series provide examples of the 3N series 1 class of compounds. The chemistry described by Meares et al. can be modified to give examples of the 3NX series of compounds.

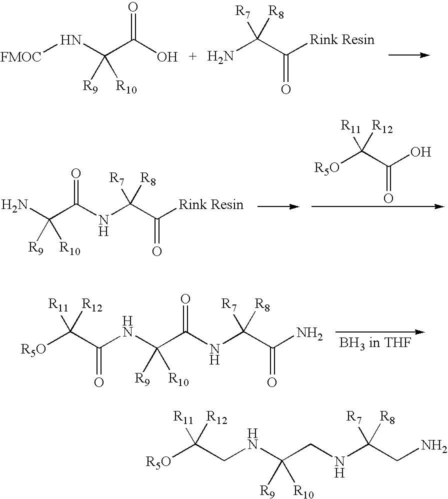

Standard peptide synthesis according to the so-called reverse Rink approach as shown above using FMOC protected natural and un-natural amino acids which can be conveniently cleaved at the penultimate step of the synthesis generates a modified tri-peptide C-terminal amide. The cases where X₄ is O are incorporated by the use of an alpha-substituted carboxylic acid in the last coupling step. This is reduced using Diborane in THF to give the open chain tetra-aza compounds.

The incorporation of R₁, R₂, R₅ and R₆ can be accomplished with this chemistry by standard procedures.

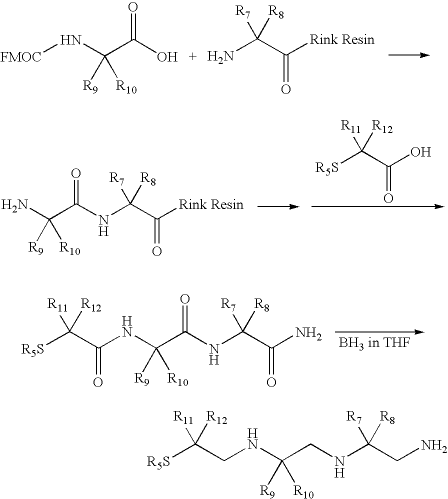

For the cases where X₄=S a similar approach using standard peptide synthesis according to the so-called reverse Rink approach as shown above can be used. Coupling with FMOC protected natural and un-natural amino acids, which can be conveniently cleaved at the penultimate step of the synthesis, generates a modified tri-peptide C-terminal amide. The incorporation of X₄=S is achieved by the use of an alpha-substituted carboxylic acid in the last coupling step. This is reduced using Diborane in THF to give the open chain tetra-aza compounds.

The incorporation of R₁, R₂, R₅ and R₆ can be accomplished with this chemistry by standard procedures.

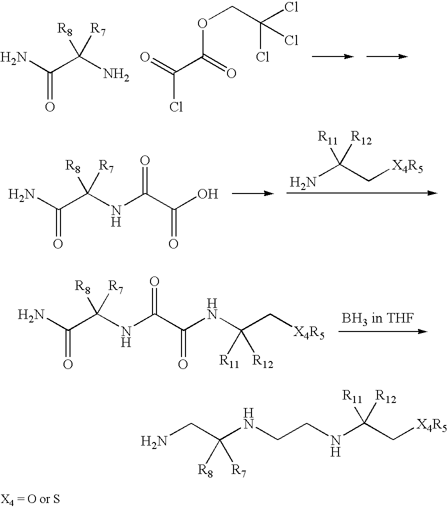

The oxalamide approach, shown above, can also lead to successful syntheses of this class of compounds, although the central substituents are always going to be hydrogen or its isotopes with this kind of chemistry. This particular variant makes use of the trichloroethyl ester group to protect one of the carboxylic acid functions of oxalic acid but other protecting groups are also envisaged. Reaction of an amino acid amide derived from a natural or unnatural amino acid with a differentially protected oxalyl mono chloride gives the mono-oxalamide shown which can be reacted under standard peptide coupling conditions with an ethanolamine or ethanethiolamine derivative to give the un-syimnetrical bis-oxalamide which can then be reduced with diborane as shown to give the desired tri-aza derivative.

3NX Series 2:

when X₁, X₂, and X₄ are N and X₃ is O or S then:

R₄ does not exist, and

R₁, R₂, R₃, R₅, and R₆ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH);

n1, n2, and n3 are independently chosen to be 2 or 3, and each repeat of any of n1, n2, and n3 may be the same as or different than any other repeat; and

R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R₁, R₂, R₃, R₅, or R₆ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmiaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

### Synthesis of Examples of the Open Chain 3NX Series 2 of Formula I

A different approach can be used for the synthesis of the 3N series 2 class of compounds. The key component is the incorporation in the synthesis of an appropriately substituted and protected ethanolamine or ethanethiolamine derivative, which is readily available from both natural and un-natural amino acids, as shown below.

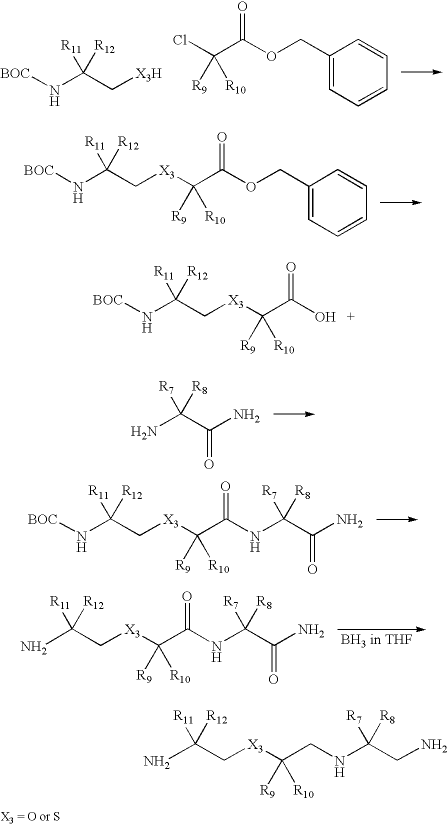

The BOC protected ethanolamine or ethanethiolamine is reacted with an appropriate benzyl protected alpha chloroacid. After hydrogenation to deprotect the ester function, standard peptide coupling with a natural or unnatural amino acid amide followed by deprotection and reduction with diborane in THF gives the open chain tri-aza compounds. If hydrogenation is not compatible with other functionality in the molecule then alternative combinations of protecting groups can be used such as trichloroethyloxy carbonyl and t-butyl.

The incorporation of R₁, R₂, R₅ and R₆ can be accomplished with this chemistry by standard procedures.

2N2X Series 1:

when X₂ and X₃ are N and X₁ and X₄ are O or S then:

R₁ and R₆ do not exist;

R₂, R₃, R₄, and R₅ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH);

n1, n2, and n3 are independently chosen to be 2 or 3, and each repeat of any of n1, n2, and n3 may be the same as or different than any other repeat; and

R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R₂, R₃, R₄, or R₅ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 allcyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

### Synthesis of Examples of the Open Chain 2N2X Series 1 of Formula I

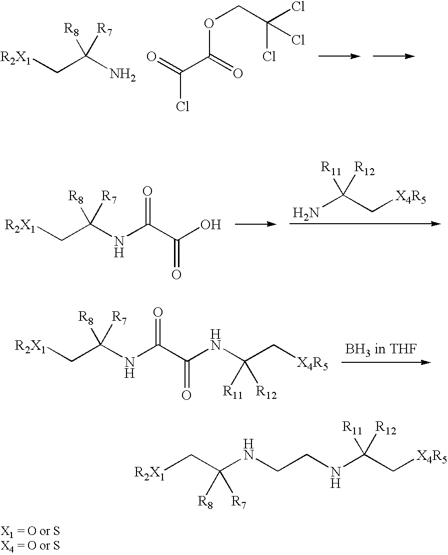

The oxalamide approach, shown above, can lead to successful syntheses of this class of compounds. This particular variant makes use of the trichloroethyl ester group to protect one of the carboxylic acid fumctions of oxalic acid but other protecting groups are also envisaged. Reaction of an aminoalcohol or aminothiol derivative readily available from a natural or unnatural amino acid with a differentially protected oxalyl mono chloride gives the mono-oxalamide shown which can be reacted under standard peptide coupling condition to give the un-symmetrical bis-oxalamide which can then be reduced with diborane to give the desired tetra-aza derivative.

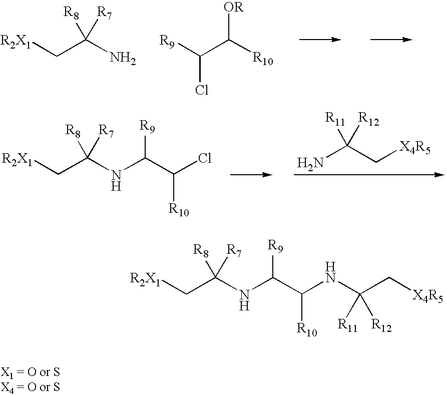

A variant of the dichloroethane approach, shown above, can also lead to successful syntheses of this class of compounds. Reaction of an aminoalcohol or aminothiol derivative readily available from a natural or unnatural amino acid with an O-protected 1-chloro, 2-hydroxy ethane derivative followed by deprotection and substitution with chloride gives the mono-chloro compound shown which can be further reacted with an appropriate aminoalcohol or aminothiol derivative readily available from a natural or unnatural amino acid to give the un-symmetrical desired product.

2N2X Series 2:

when X₁ and X₃ are N and X₂ and X₄ are O or S then:

R₃ and R₆ do not exist;

R₁, R₂, R₄, and R₅ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH);

n1, n2, and n3 are independently chosen to be 2 or 3, and each repeat of any of n1, n2, and n3 may be the same as or different than any other repeat; and

R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R₁, R₂, R₄, or R₅ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

### Synthesis of the Open Chain 2N2X Series 2 of Formula I

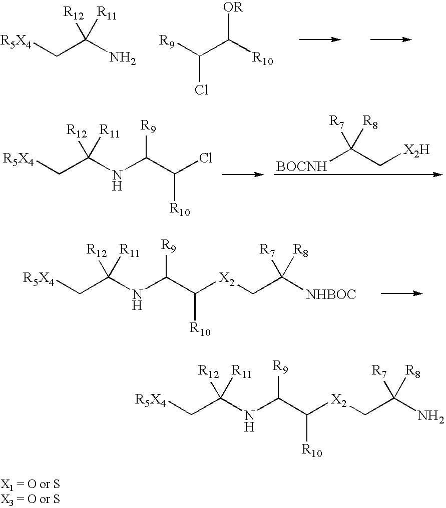

A variant of the dichloroethane approach, shown above, can lead to successful syntheses of this class of compounds. Reaction of an aminoalcohol or aminothiol derivative readily available from a natural or unnatural amino acid with an O-protected 1-chloro, 2-hydroxy ethane derivative followed by deprotection and substitution with chloride gives the mono-chloro compound shown which can be further reacted with an appropriately protected aminoalcohol or aminothiol derivative, readily available from a natural or unnatural amino acid, to give the un-symmetrical desired product after de-protection.

2N2X Series 3:

when X₁ and X₂ are N and X₃ and X₄ are O or S then:

R₄ and R₆ do not exist;

R₁, R₂, R₃, and R₅ are independently chosen from H, CH₃,-C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH);

n1, n2, and n3 are independently chosen to be 2 or 3, and each repeat of any of n1, n2, and n3 may be the same as or different than any other repeat; and

R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R₁, R₂, R₃, or R₅ may be functionalized for attachment, for example, to peptides, proteins, polyethylene glycols and other such chemical entities in order to modify the overall pharmaco-kinetics, deliverability and/or half lives of the constructs. Examples of such flnctionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

### Synthesis of the Open Chain 2N2X Series 3

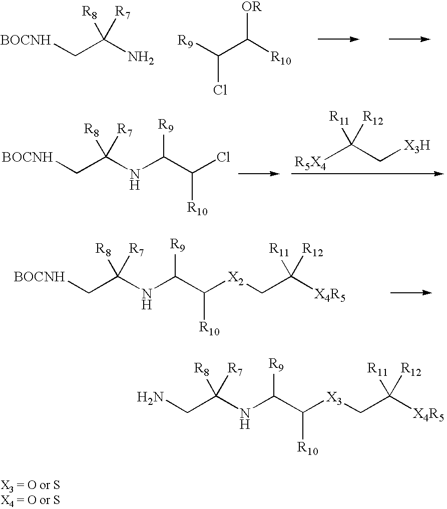

A variant of the dichloroethane approach, shown above, can lead to successful syntheses of this class of compounds. Reaction of a monoprotected ethylene diamine derivative, readily available from a natural or unnatural amino acid with an O-protected 1-chloro, 2-hydroxy ethane derivative followed by deprotection and substitution with chloride gives the mono-chloro compound shown which can be further reacted with an appropriately protected bis-alcohol or bis thiol derivative, readily available from a natural or unnatural amino acid, to give the un-symmetrical desired product after de-protection.

2N2X Series 4:

when X₁ and X₄ are N and X₂ and X₃ are O or S then:

R₃ and R₄ do not exist;

R₁, R₂, R₅ and R₆ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH);

n1, n2, and n3 are independently chosen to be 2 or 3, and each repeat of any of n1, n2, and n3 may be the same as or different than any other repeat; and

R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R₁, R₂, R₅, or R₆ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

### Synthesis of the Open Chain 2N2X Series 4 of Formula I

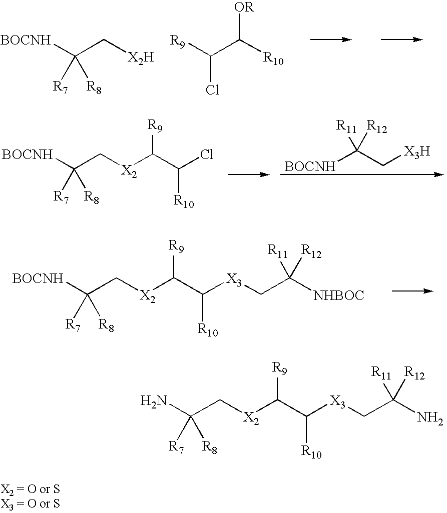

A variant of the dichloroethane approach, shown above, can lead to successful syntheses of this class of compounds. Reaction of an appropriately protected bis-alcohol or bis thiol derivative, readily available from a natural or unnatural amino acid, with an O-protected 1-chloro, 2-hydroxy ethane derivative followed by deprotection and substitution with chloride gives the mono-chloro compound shown which can be further reacted with an appropriately protected bis-alcohol or bis thiol derivative, readily available from a natural or unnatural amino acid, to give the un-symmetrical desired product after de-protection.

For the Tetra-Heteroatom Cyclic Series:

One of R₁ and R₂ (if R₁ does not exist) and one of R₅ (if R₆ does not exist) and R₆ are joined together to form the bridging group (CR₁₃R₁₄)n4;

X₁, X₂, X₃, and X₄ are independently chosen from the atoms N, S or O such that:

4N Macrocyclic Series:

when X₁, X₂, X₃, and X₄ are N then:

R₂, R₃, R₄, and R₅ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH) ₂, CH₂P(CH₃)O(OH);

n1, n2, n3, and n4 are independently chosen to be 2 or 3, and each repeat of any of n1, n2, n3 and n4 may be the same as or different than any other repeat; and

R₇, R₈, R₉, R₁₀, R₁₁, R₁₂, R₁₃ and R₁₄ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R₂, R₃, R₄, or R₅ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R₇, R₈, R₉, R₁₀, R₁₁, R₁₂, R₁₃ or R₁₄ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

### Synthesis of Examples of the Macrocyclic 4N Series of Formula I

Triethylenetetramine itself has been synthesized by reaction of 2 equivalents of ethylene diamine with 1,2-dichloro ethane to give triethylenetetramine directly. Possible side products from this synthesis include the 12N4 macrocycle shown below, which could also be synthesized directly from Triethylenetetramine by reaction with a further equivalent of 1,2-dichloro ethane under appropriately dilute concentrations to provide the 12N4 macrocycle shown. Modification of this procedure by using starting materials with appropriate Rₐ and R_{b} (where Rₐ, R_{b} correspond to R₇, R₈ or R₁₁, R₁₂) groups would lead to symmetrically substituted 12N4 macrocycle examples as shown below:

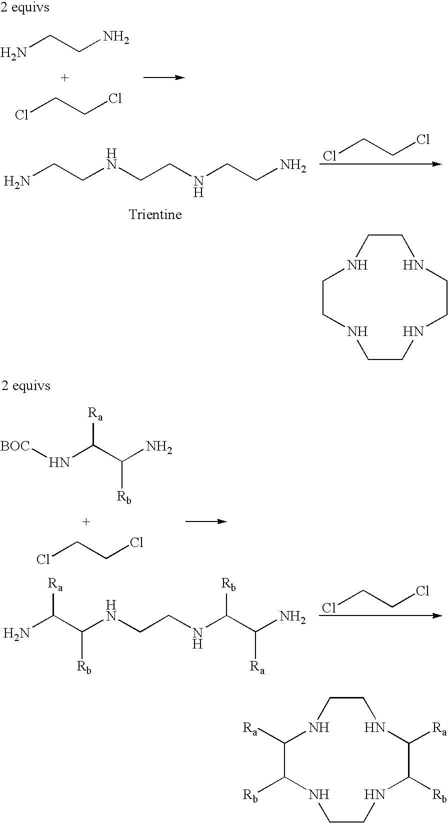

The judicious use of protecting group chemistry such as the widely used BOC (t-butyloxycarbonyl) group allows the chemistry to be directed specifically towards the substitution pattern shown. Other approaches such as via the chemistry of ethyleneimine may also lead to a subset of the tetra-aza series. In order to obtain the un-symmetrically substituted derivatives a variant of some chemistry described by Meares et al. should be used. Standard peptide synthesis using the Merrifield approach or the SASRIN resin along with FMOC protected natural and un-natural amino acids which can be conveniently cleaved at a later step of the synthesis generates a fully protected tetra-peptide C-terminal SASRIN derivative. Cleavage of the N terminal FMOC protecting group followed by direct cyclization upon concomitant cleavage from the resin gives the macrocyclic tetrapeptide. This is reduced using Diborane in THF to give the 12N4 series of compounds as shown below:

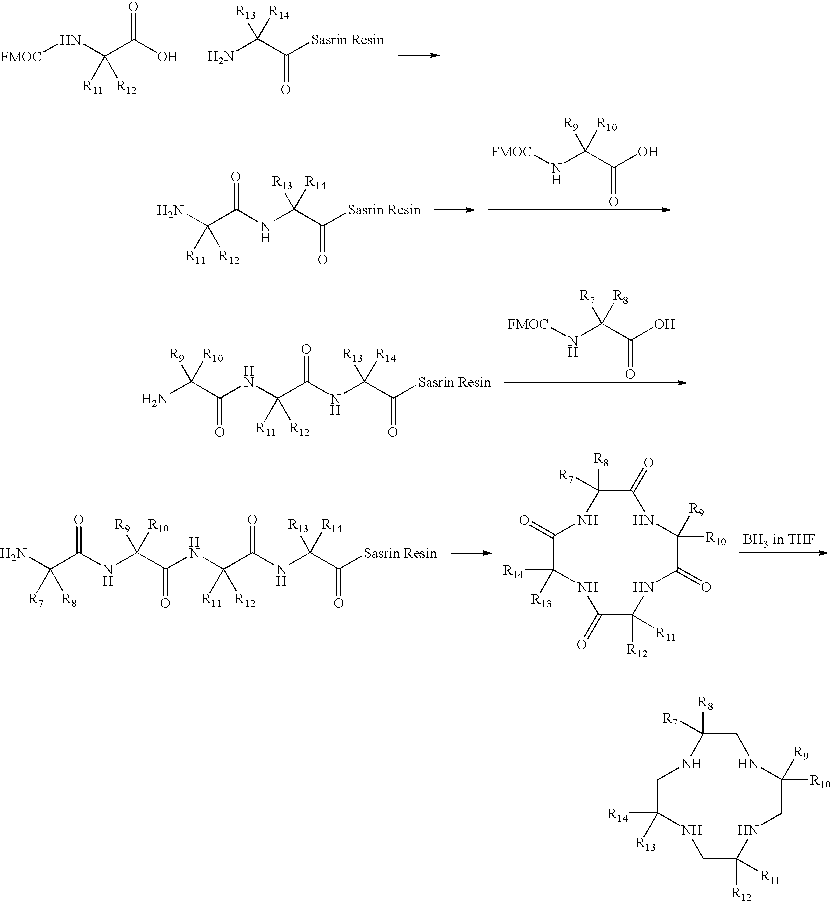

The incorporation of R₁, R₂, R₅ and R₆ can be accomplished with this chemistry by standard procedures.

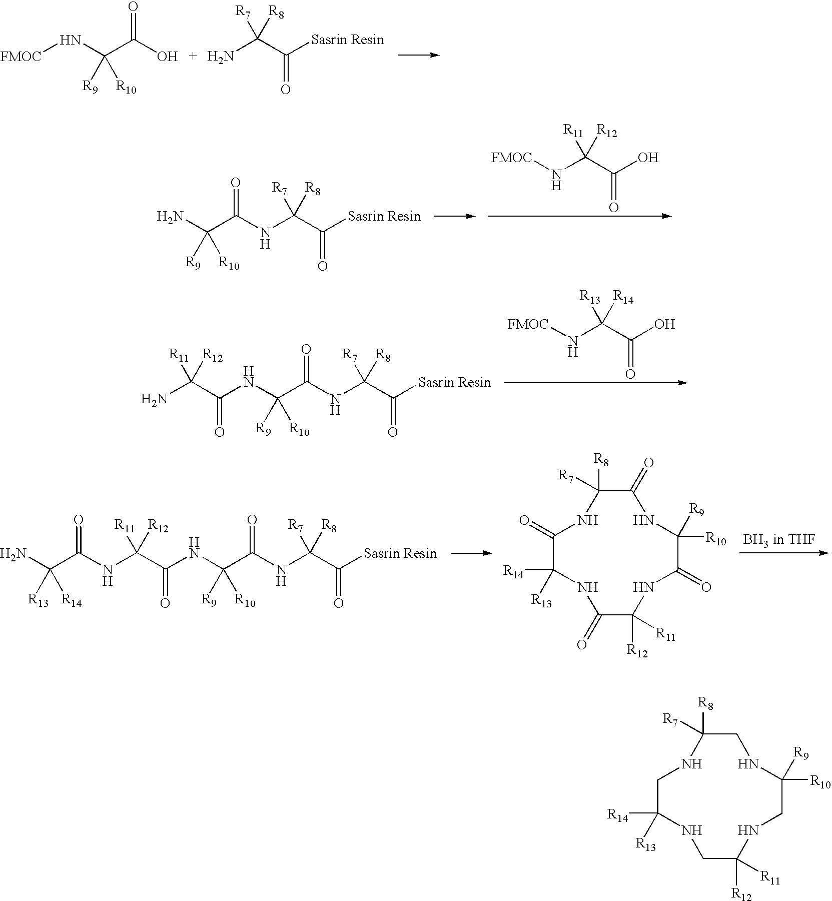

The reverse Merrifield/SASRIN approach, shown above, also leads to this class of tetra-aza derivatives and may be useful in cases where peptide coupling of a sterically hindered amino acid requires multiple coupling attempts in order to achieve success in the initial Merrifield approach.

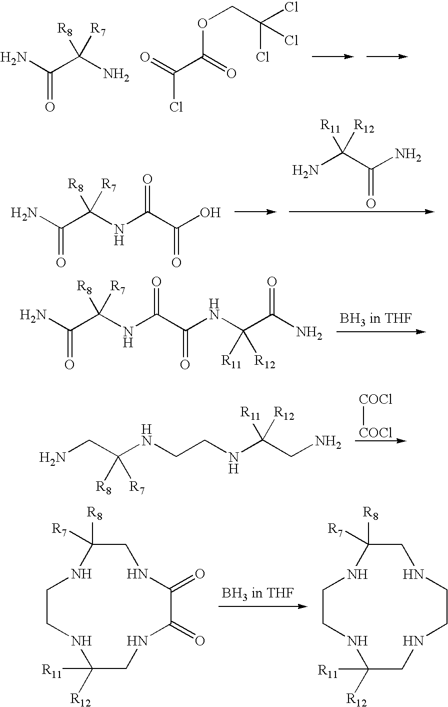

The oxalamide approach, shown above, also can lead to successful syntheses of this class of compounds. This particular variant makes use of the trichloroethyl ester group to protect one of the carboxylic acid functions of oxalic acid but other protecting groups are also envisaged. Reaction of an amino acid amide derived from a natural or unnatural amino acid with a differentially protected oxalyl mono chloride gives the mono-oxalamide shown which can be reacted under standard peptide coupling condition to give the un-symmetrical bis-oxalamide which can then be reduced with diborane to give the desired tetra-aza derivative. Further reaction with oxalic acid gives the cyclic derivative, which can then be reduced once again with diborane to give the 12N4 series of compounds.

3NX Series:

when X₁, X₂, X₃, are N and X₄ is S or O then:

R₅ does not exist;

R₂, R₃, and R₄ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri,-tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH);

n1, n2, n3, and n4 are independently chosen to be 2 or 3, and each repeat of any of n1, n2, n3 and n4 may be the same as or different than any other repeat; and

R₇, R₈, R₉, R₁₀, R₁₁, R₁₂, R₁₃ and R₁₄ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R₂, R₃ or R₄ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R₇, R₈, R₉, R₁₀, R₁₁, R₁₂, R₁₃ or R₁₄ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

### Synthesis of Examples of the Macrocyclic 3NX Series of Formula I

Triethylenetetramine itself has been synthesized by reaction of 2 equivalents of ethylene diamine with 1,2-dichloro. ethane to give triethylenetetramine directly. Possible side products from this synthesis include the 12N4 macrocycle shown below, which could also be synthesized directly from Triethylenetetramine by reaction with a further equivalent of 1,2-dichloro ethane under appropriately dilute concentrations to provide the 12N4 macrocycle shown. Modification of this procedure by using starting materials with appropriate R groups leads to symmetrically substituted 12N4 macrocycle examples as shown below:

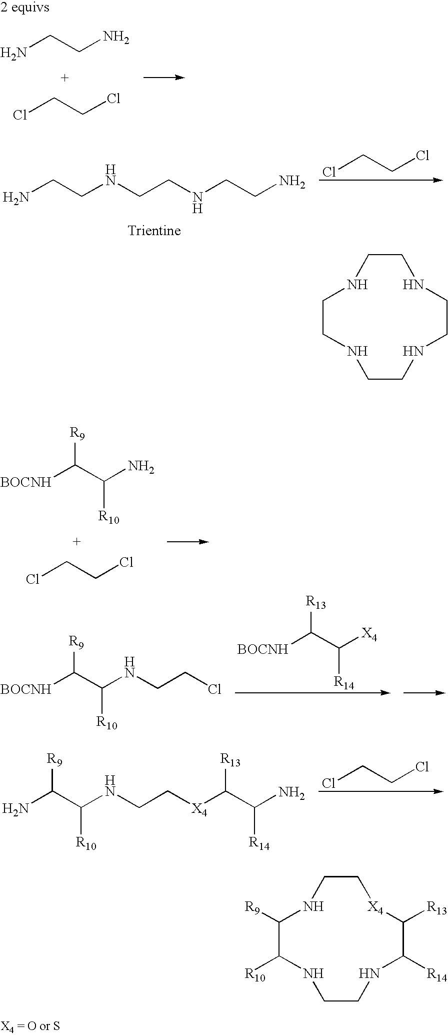

The judicious use of protecting group chemistry such as the widely used BOC (t-butyloxycarbonyl) group allows the chemistry to be directed specifically towards the substitution pattern shown. Other approaches such as via the chemistry of ethyleneimine may also lead to a subset of the tri-aza X series. In order to obtain alternative un-symmetrically substituted derivatives a variant of some chemistry described by Meares et al. could be used. Standard peptide synthesis using the Merrifield approach or the SASRIN resin along with FMOC protected natural and un-natural amino acids which can be conveniently cleaved at a later step of the synthesis generates a tri-peptide C-terminal SASRIN derivative which can be further elaborated with an appropriate BOCO or BOCS compound the give the resin bound 3NX compound shown. Reduction with diborane followed by Tosylation would give the 3NX OTosyl linear compound, which, upon deprotection and cyclization would give the desired 3NX macrocycle as shown below:

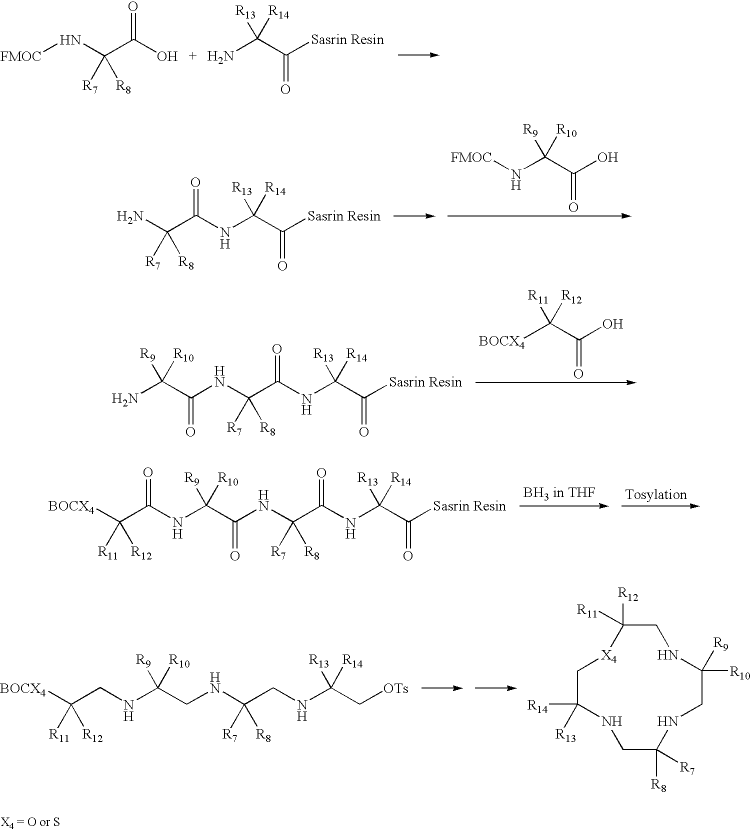

The incorporation of R₁, R₂, R₅ and R₆ can be accomplished with this chemistry by standard procedures.

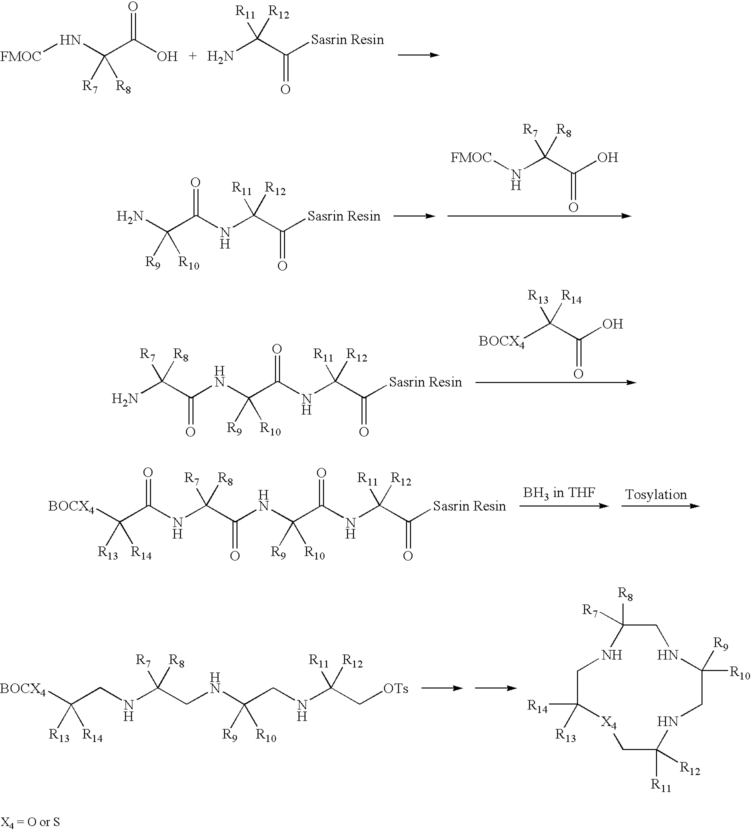

The reverse Merrifield/SASRIN approach, shown above, also leads to this class of tetra-aza derivatives and may be useful in cases where peptide coupling of a sterically hindered amino acid requires multiple coupling attempts in order to achieve success in the initial Merrifield approach.

2N2X Series 1:

when X₂ and X₃ are N and X₁ and X₄ are O or S then:

R₂ and R₅ do not exist

R₃ and R₄ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH);

n1, n2, n3, and n4 are independently chosen to be 2 or 3, and each repeat of any of n1, n2, n3 and n4 may be the same as or different than any other repeat; and

R₇, R₈, R₉, R₁₀, R₁₁, R₁₂, R₁₃ and R₁₄ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl

In addition, one or both of R₃, or R₄ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

Furthermore one or several of R₇, R₈, R₉, R₁₀, R₁₁, R₁₂, R₁₃ or R₁₄ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

### Synthesis of Examples of the Macrocyclic 2N2X Series 1 of Formula I

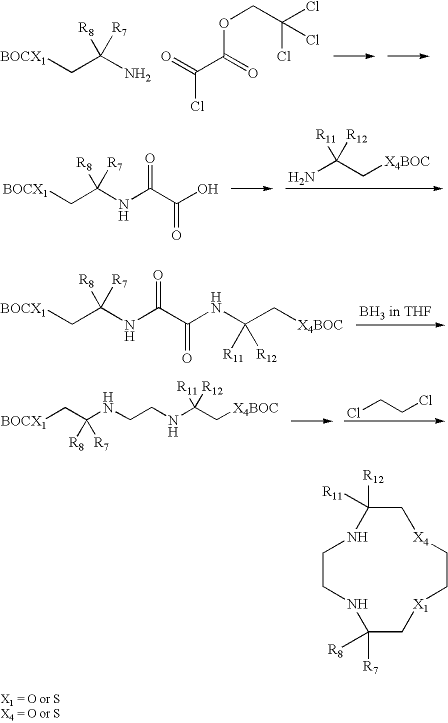

The oxalamide approach, shown above, again can lead to successful syntheses of this class of compounds, although the central substituents are always going to be hydrogen or its isotopes with this kind of chemistry. This particular variant makes use of the trichloroethyl ester group to protect one of the carboxylic acid functions of oxalic acid but other protecting groups are also envisaged. Reaction of an aminoalcohol or aminothiol derivative readily available from a natural or unnatural amino acid with a differentially protected oxalyl mono chloride gives the mono-oxalamide shown which can be reacted under standard peptide coupling condition to give the un-symmetrical bis-oxalamide which can then be reduced with diborane to give the desired di-aza derivative. Deprotection followed by cyclization would give the 12N2X2 analogs.

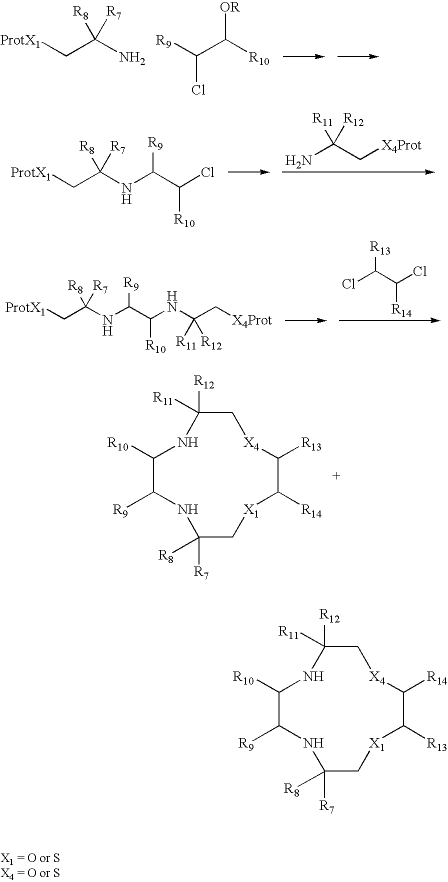

A variant of the dichloroethane approach, shown above, can also lead to successful syntheses of this class of compounds. Reaction of an aminoalcohol or aminothiol derivative readily available from a natural or imnatural amino acid with an O-protected 1-chloro, 2-hydroxy ethane derivative followed by deprotection and substitution with chloride gives the mono-chloro compound shown which can be further reacted with an appropriate aminoalcohol or aminothiol derivative readily available from a natural or unnatural amino acid to give the un-symmetrical product shown. Deprotection followed by cyclization with a dichloroethane derivative would give a mixture of the the two position isomers shown.

2N2X Series 2:

when X₁ and X₃ are N and X₂ and X₄ are O or S then:

R₃ and R₅ do not exist

R₂ and R₄ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂2PO(OH)₂, CH₂P(CH₃O(OH);

n1, n2, n3, and n4 are independently chosen to be 2 or 3, and each repeat of any of n1, n2, n3 and n4 may be the same as or different than any other repeat; and

R₇, R₈, R₉, R₁₀, R₁₁, R₁₂, R₁₃ and R₁₄ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or both of R₂, or R₄ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

Furthermore one or several of R₇, R₈, R₉, R₁₀, R₁₁, R₁₂, R₁₃ or R₁₄ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

### Synthesis of Examples of the Macrocyclic 2N2X Series 2 of Formula I

Triethylenetetramine itself has been synthesized by reaction of 2 equivalents of ethylene diamine with 1,2-dichloro ethane to give triethylenetetramine directly. Possible side products from this synthesis include the 12N4 macrocycle shown below, which could also be synthesized directly from Triethylenetetramine by reaction with a further equivalent of 1,2-dichloro ethane under appropriately dilute concentrations to provide the 12N4 macrocycle shown. Modification of this procedure by using starting materials with appropriate R groups would lead to symmetrically substituted 12N4 macrocycle examples as shown below:

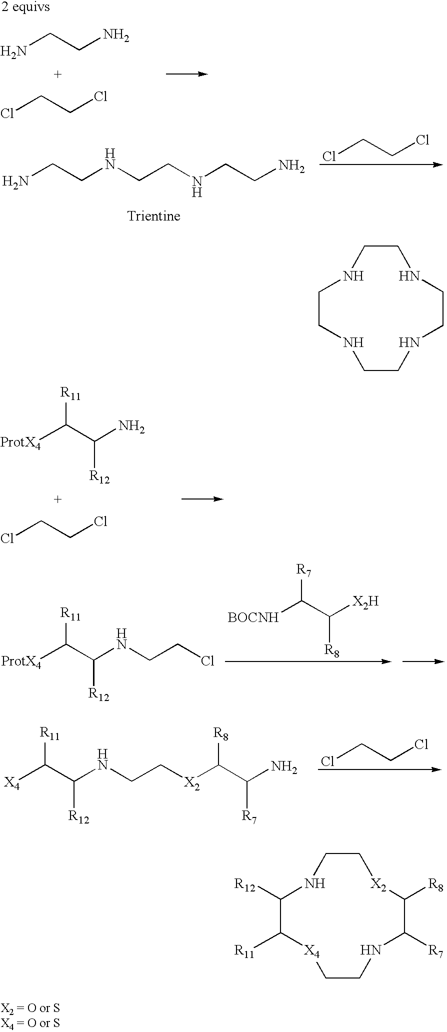

The judicious use of protecting group chemistry such as the widely used BOC (t-butyloxycarbonyl) group and an appropriate O or S protecting group allows the chemistry to be directed specifically towards the substitution pattern shown. Other approaches such as via the chemistry of ethyleneimine may also lead to a subset of the di-aza 2X series. A variant of this approach using substituted dichloroethane derivatives could be used to access more complex substitution patterns. This would lead to mixtures of position isomers, which can be separated by HPLC.

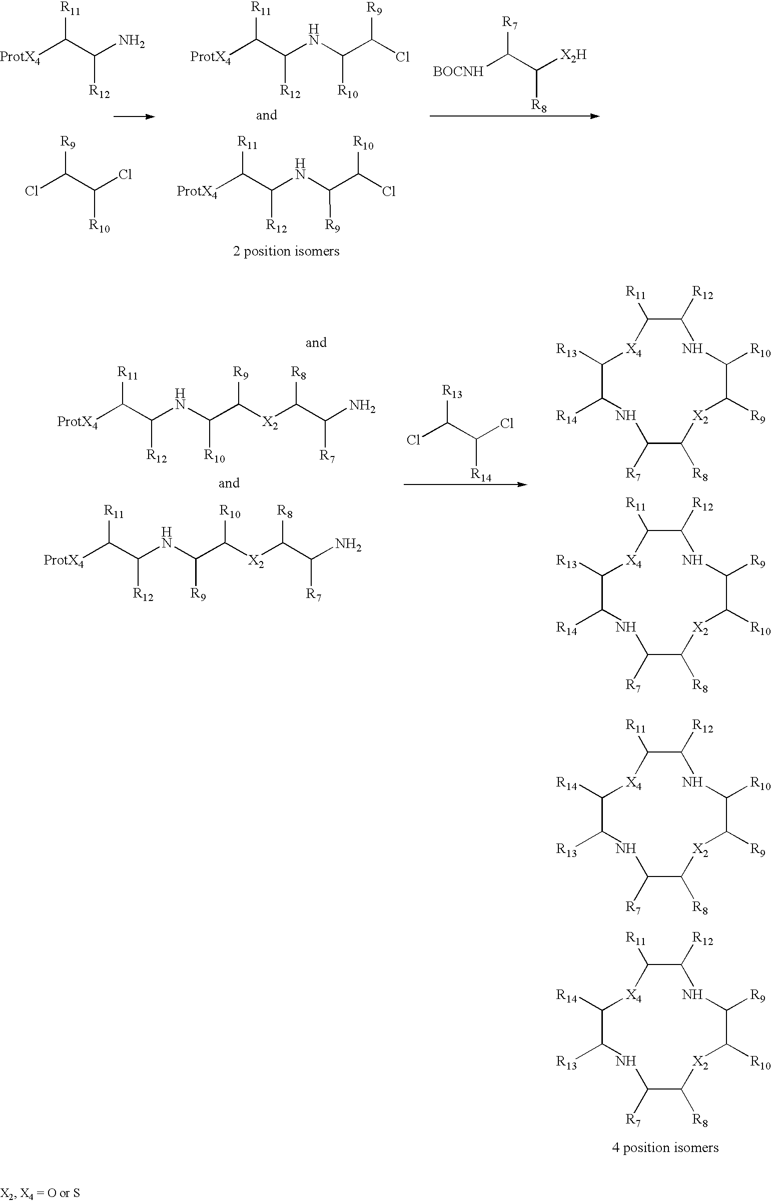

1N3X Series:

when X₁ is N and X₂, X₃ and X₄ are O or S then:

R₃, R₄ and R₅ do not exist;

R₂ is independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH);

n1, n2, n3, and n4 are independently chosen to be 2 or 3, and each repeat of any of n1, n2, n3 and n4 may be the same as or different than any other repeat; and

R₇, R₈, R₉, R₁₀, R₁₁, R₁₂, R₁₃ and R₁₄ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, R₂ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R₇, R₈, R₉, R₁₀, R₁₁, R₁₂, R₁₃ or R₁₄ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

### Synthesis of Examples of the Macrocyclic 1N3X Series of Formula I

Triethylenetetramine itself has been synthesized by reaction of 2 equivalents of ethylene diamine with 1,2-dichloro ethane to give triethylenetetramine directly. Possible side products from this synthesis include the 12N4 macrocycle shown below, which could also be synthesized directly from Triethylenetetramine by reaction with a further equivalent of 1,2-dichloro ethane under appropriately dilute concentrations to provide the 12N4 macrocycle shown. Modification of this procedure by using starting materials with appropriate R groups would lead to substituted 12NX3 macrocycle examples as shown below:

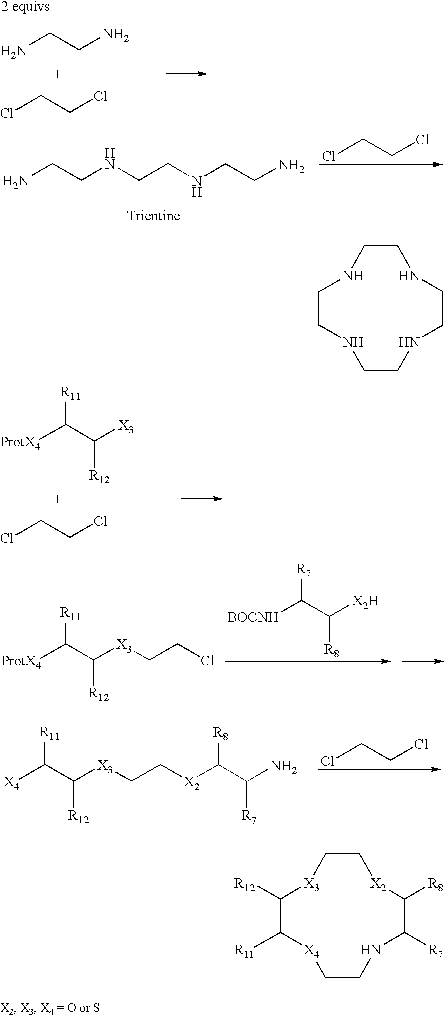

The judicious use of protecting group chemistry such as the widely used BOC (t-butyloxycarbonyl) group and an appropriate O or S protecting group allows the chemistry to be directed specifically towards the substitution pattern shown. Other approaches such as via the chemistry of ethyleneimine may also lead to a subset of the mono-aza 3X series. A variant of this approach using substituted dichloroethane derivatives could be used to access more complex substitution patterns. This would lead to mixtures of position isomers, which can be separated by HPLC.

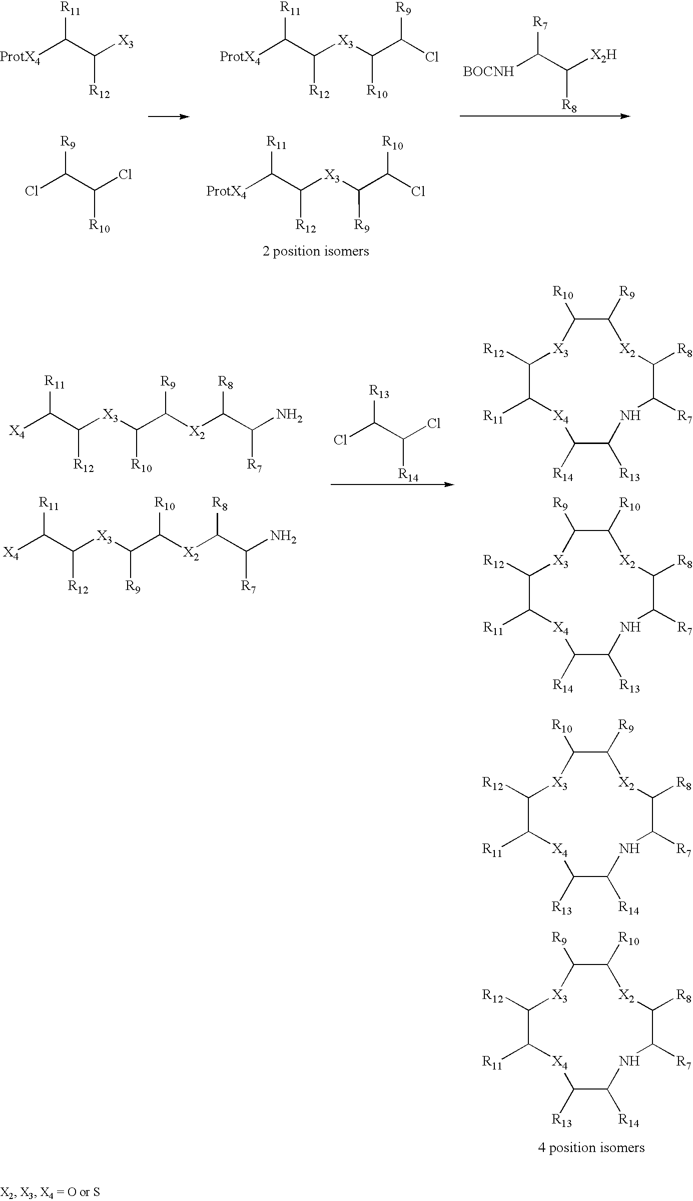

For the Tri-Heteroatom Acyclic Examples of Formula II:

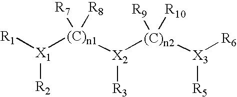

X₁, X₂, and X₃ are independently chosen from the atoms N, S or O such that:

3N Series:

when X₁, X₂, and X₃ are N then:

R₁, R₂, R₃, R₅, and R₆ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH);

n1 and n2 are independently chosen to be 2 or 3, and each repeat of any of n1 and n2 may be the same as or different than any other repeat; and

R₇, R₈, R₉, and R₁₀ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R₁, R₂, R₃, R₅ or R₆ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

Furthermore one or several of R₇, R₈, R₉, or R₁₀ may be finctionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyi-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

### Synthesis of the Open Chain 3N Series of Formula II

As mentioned above Triethylenetetramine itself has been synthesized by reaction of 2 equivalents of ethylene diamine with 1,2-dichloro ethane to give Triethylenetetramine directly. A variant of this procedure by using starting materials with appropriate R groups and 1-amino,2-chloro ethane would lead to some open chain 3N examples as shown below:

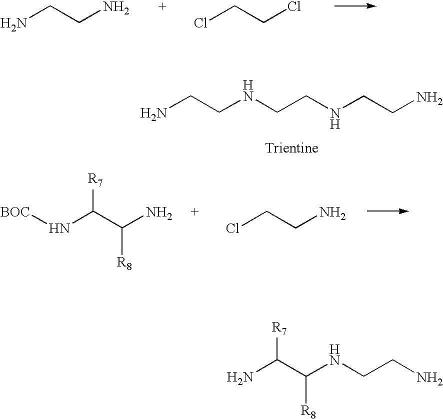

The judicious use of protecting group chemistry such as the widely used BOC (t-butyloxycarbonyl) group allows the chemistry to be directed specifically towards the substitution pattern shown. Other approaches such as via the chemistry of ethyleneimine may also lead to a subset of the tri-aza series. In order to obtain the un-symmetrically substituted derivatives a variant of some chemistry described by Meares et al. could be used. Standard peptide synthesis using the Rink resin along with FMOC protected natural and un-natural amino acids which can be conveniently cleaved at the penultimate step of the synthesis generates a di-peptide C-terminal amide. This can be reduced using Diborane in THF to give the open chain tri-aza compounds as shown below:

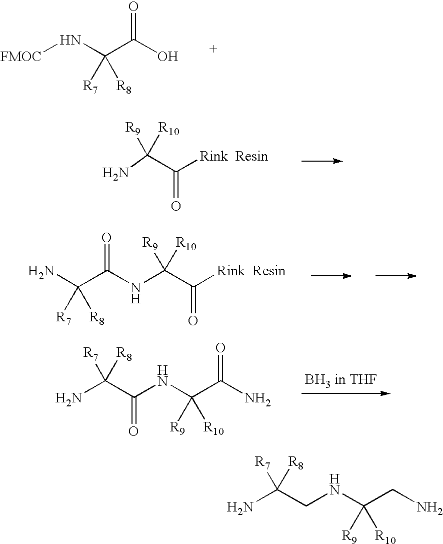

The reverse Rink approach may also be useful where peptide coupling is slowed for a particular substitution pattern as shown below. Again the incorporation of R₁, R₂, R₅ and R₆ can be accomplished with this chemistry by standard procedures:

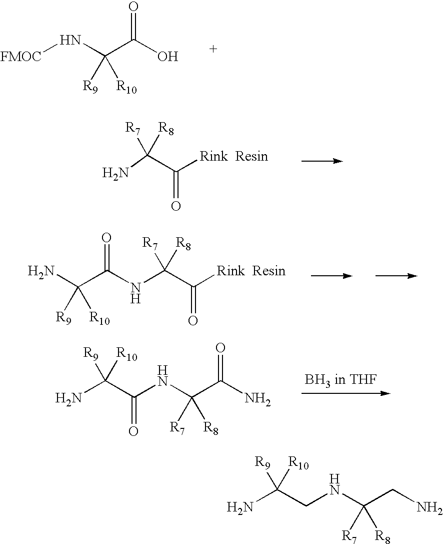

2NX Series 1:

when X₁ and X₃ are N and X₂ is S or O then:

R₃ does not exist

R₁, R₂, R₅, and R₆ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH);

n1 and n2 are independently chosen to be 2 or 3, and each repeat of any of n1 and n2 may be the same as or different than any other repeat; and

R₇, R₈, R₉, and R₁₀ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl

In addition, one or several of R₁, R₂, R₅ or R₆ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R₇, R₈, R₉, or R₁₀ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

### Synthesis of the Open Chain 2NX Series 1 of Formula II

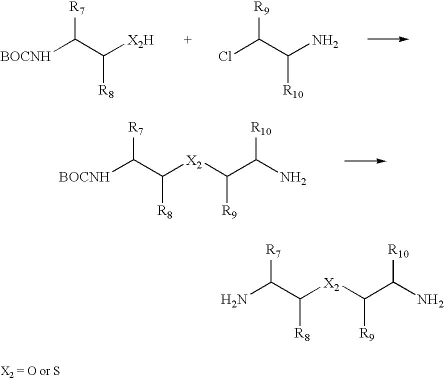

The synthesis of the 2NX series 1 compounds can be readily achieved as shown above. The judicious use of protecting group chemistry such as the widely used BOC (t-butyloxycarbonyl) group allows the chemistry to be directed specifically towards the substitution pattern shown above. Other approaches such as via the chemistry of ethyleneimine may also lead to a subset of the tri-aza X series.

2NX Series 2

when X₁ and X₂ are N and X₃ is O or S then:

R₅ does not exist;

R₁, R₂, R₃ and R₆ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH);

n1 and n2 are independently chosen to be 2 or 3, and each repeat of any of n1 and n2 may be the same as or different than any other repeat; and

R₇, R₈, R₉, and R₁₀ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R₁, R₂, R₅, or R₆ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R₇, R₈, R₉, or R₁₀ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

### Synthesis of the Open Chain 2NX Series 2 of Formula II

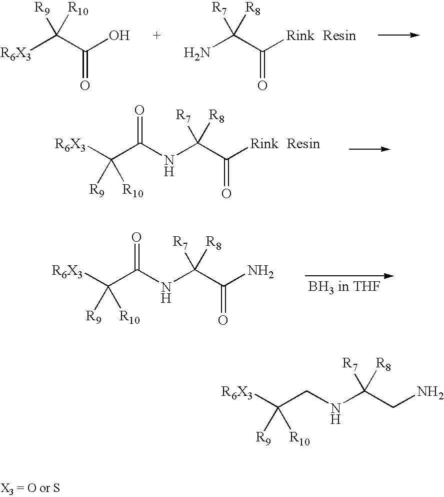

For the cases where X₃=O or S a similar approach using standard peptide synthesis according to the Rink approach as shown above can be used. Coupling of a suitably protected alpha thiolo or hydroxy carboxylic acid with a Rink resin amino acid derivative followed by cleavage gives the desired linear di-amide, which can be reduced with Diborane in THF to give the open chain 2NX compounds.

The incorporation of R₁, R₂, R₅ and R₆ can be accomplished with this chemistry by standard procedures.

The reverse Rink version is also feasible and again the incorporation of R₁, R₂, R₅ and R₆ can be accomplished with this chemistry by standard procedures.

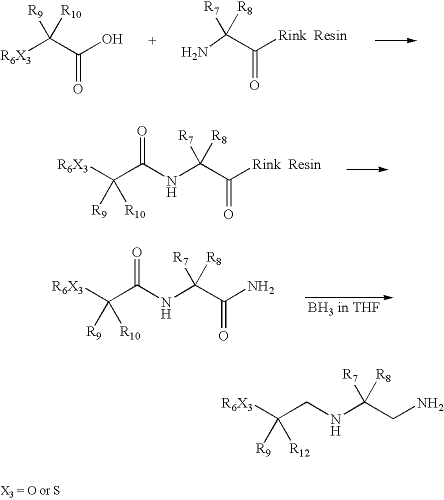

Tri-Heteroatom Cyclic Series of Formula II:

R₁ and R₆ form a bridging group (CR₁₁R₁₂)n3; and

X₁, X₂, and X₃ are independently chosen from the atoms N, S or O such that:

3N Series:

when X₁, X₂ and X₃ are N then:

R₂, R₃, and R₅ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH);

n1, n2, and n3 are independently chosen to be 2 or 3, and each repeat of any of n1, n2 and n3 may be the same as or different than any other repeat; and

R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or several of R₂, R₃, or R₅ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, C1-C10 alkyl-S-protein.

### Synthesis of Examples of the Macrocyclic 3N Series of Formula II

As mentioned above Triethylenetetramine itself has been synthesized by reaction of 2 equivalents of ethylene diamine with 1,2-dichloro ethane to give Triethylenetetramine directly. A variant of this procedure by using starting materials with appropriate R groups and 1-amino,2-chloro ethane would lead to open chain 3N examples which could then be cyclized by reaction with an appropriate 1,2 dichloroethane derivative as shown below:

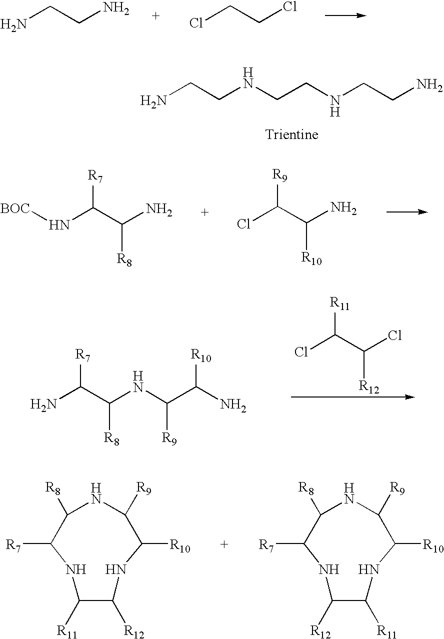

The judicious use of protecting group chemistry such as the widely used BOC (t-butyloxycarbonyl) group allows the chemistry to be directed specifically towards the substitution pattern shown. Other approaches such as via the chemistry of ethyleneimine may also lead to a subset of the macrocyclic tri-aza series. In order to obtain the un-symmetrically substituted derivatives a variant of some chemistry described by Meares et al. could be used. Standard peptide synthesis using the Merrifield approach/SASRIN resin along with FMOC protected natural and un-natural amino acids which can be conveniently cleaved at the penultimate step of the synthesis generates a tri-peptide attached to resin via it's C-terminus. This can be cyclized during concomitant cleavage from the resin followed by reduction using Diborane in THF to give the cyclic tri-aza compounds as shown below:

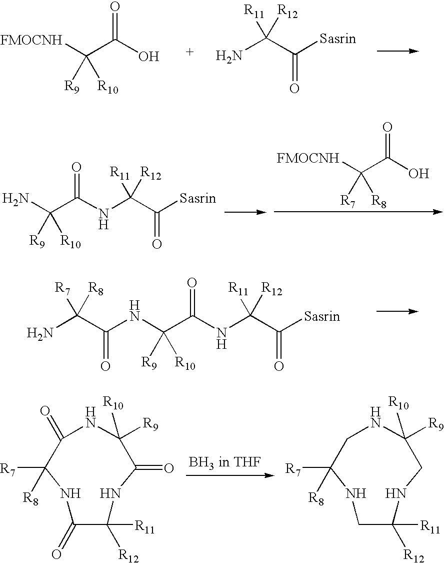

The incorporation of R₁, R₂, and R₅ can be accomplished with this chemistry by standard procedures.

The reverse Rink approach may also be useful where peptide coupling is slowed for a particular substitution pattern as shown below. Again the incorporation of R₁, R₂, R₅ and R₆ can be accomplished with this chemistry by standard procedures:

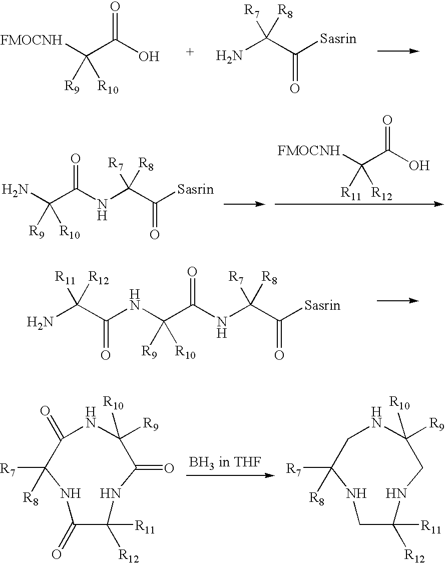

2NX Series:

when X₁ and X₂ are N and X₃ is S or O then:

R₅ does not exist;

R₂ and R₃ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH);

n1, n2, and n3 are independently chosen to be 2 or 3, and each repeat of any of n1, n2 and n3 may be the same as or different than any other repeat; and

R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, one or both of R₂ or R₃ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

### Synthesis of Examples of the Macrocyclic 2NX Series of Formula II

As mentioned above Triethylenetetramine itself has been synthesized by reaction of 2 equivalents of ethylene diamine with 1,2-dichloro ethane to give Triethylenetetramine directly. A variant of this procedure by using starting materials with appropriate R groups and 1-amino,2-chloro ethane would lead to open chain 2NX examples which could then be cyclized by reaction with an appropriate 1,2 dichloroethane derivative as shown below:

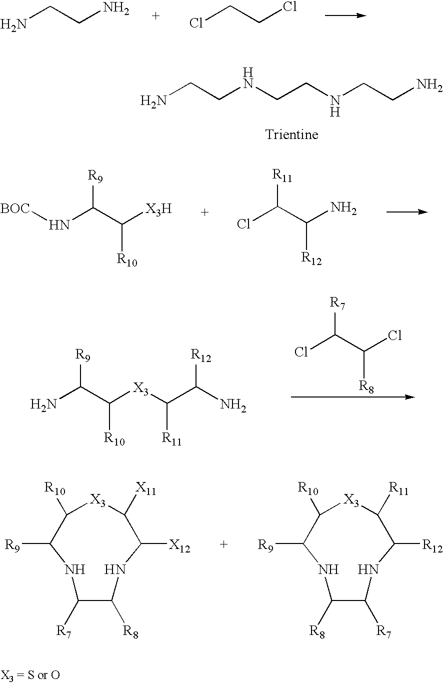

The judicious use of protecting group chemistry such as the widely used BOC (t-butyloxycarbonyl) group allows the chemistry to be directed specifically towards the substitution pattern shown. Other approaches such as via the chemistry of ethyleneimine may also lead to a subset of the macrocyclic di-aza X series. In order to obtain the un-symmetrically substituted derivatives a variant of some chemistry described by Meares et al. could be used. Standard peptide synthesis using the Merrifield approach/SASRIN resin along with FMOC protected natural and un-natural amino acids which can be conveniently cleaved at the penultimate step of the synthesis generates a tri-peptide attached to resin via it's C-terminus. This can be cyclized during concomitant cleavage from the resin followed by reduction using Diborane in THF to give the cyclic tri-aza compounds as shown below:

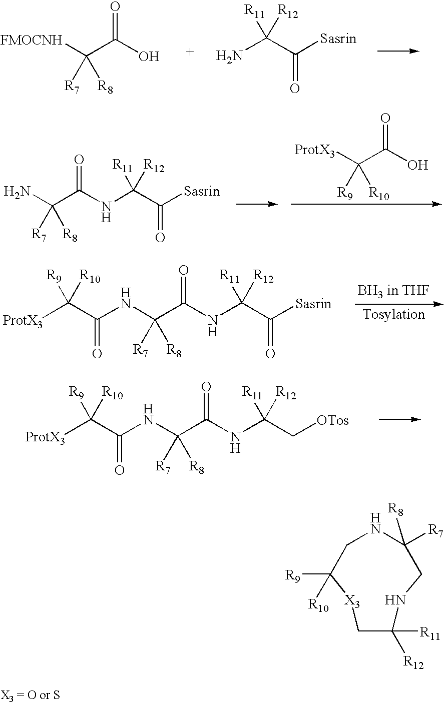

The incorporation of R₁, and R₂ can be accomplished with this chemistry by standard procedures.

The reverse Rink approach may also be useful where peptide coupling is slowed for a particular substitution pattern as shown below. Again the incorporation of R₁, and R₂ can be accomplished with this chemistry by standard procedures:

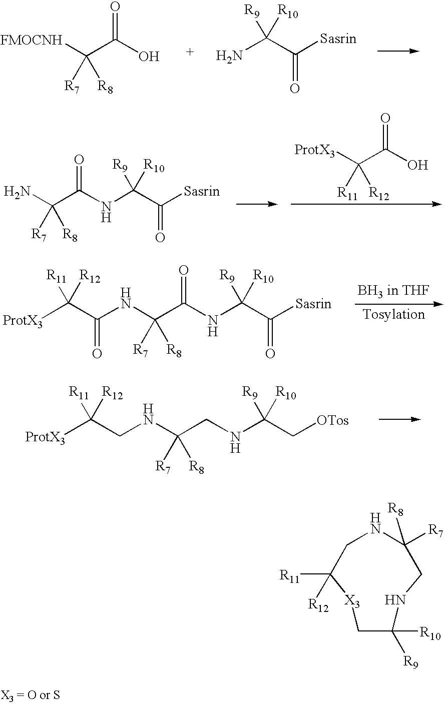

1N2X Series:

when X₁ is N and X₂ and X₃ are O or S then:

R₃ and R₅ do not exist;

R₂ is independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl, CH₂COOH, CH₂SO₃H, CH₂PO(OH)₂, CH₂P(CH₃)O(OH);

n1, n2, and n3 are independently chosen to be 2 or 3, and each repeat of any of n1, n2 and n3 may be the same as or different than any other repeat;

R₇, R₈, R₉, R₁₀, R₁₁, and R₁₂ are independently chosen from H, CH₃, C2-C10 straight chain or branched alkyl, C3-C10 cycloalkyl, C1-C6 alkyl C3-C10 cycloalkyl, aryl, mono, di, tri, tetra and penta substituted aryl, heteroaryl, fused aryl, C1-C6 alkyl aryl, C1-C6 alkyl mono, di, tri, tetra and penta substituted aryl, C1-C5 alkyl heteroaryl, C1-C6 alkyl fused aryl.

In addition, R₂ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

Furthermore one or several of R₇, R₈, R₉, R₁₀, R₁₁, or R₁₂ may be functionalized for attachment, for example, to peptides, proteins, polyethylene glycols and other such chemical entities in order to modify the overall pharmaco-kinetics, deliverability and/or half lives of the constructs. Examples of such functionalization include but are not limited to C1-C10 alkyl-CO-peptide, C1-C10 alkyl-CO-protein, C1-C10 alkyl-CO-PEG, C1-C10 alkyl-NH-peptide, C1-C10 alkyl-NH-protein, C1-C10 alkyl-NH—CO-PEG, C1-C10 alkyl-S-peptide, and C1-C10 alkyl-S-protein.

### Synthesis of Examples of the Macrocyclic 1N2X Series of Formula II

As mentioned above Triethylenetetramine itself has been synthesized by reaction of 2 equivalents of ethylene diamine with 1,2-dichloro ethane to give Triethylenetetramine directly. A variant of this procedure by using starting materials with appropriate R groups and 1-amino,2-chloro ethane would lead to open chain 1N2X examples which could then be cyclized by reaction with an appropriate 1,2 dichloroethane derivative as shown below:

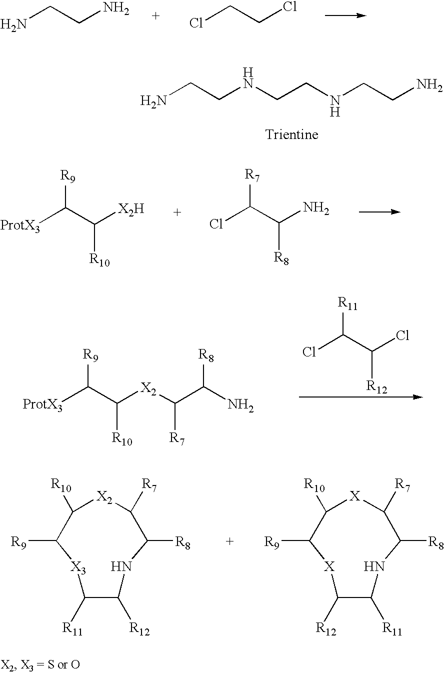

The judicious use of protecting group chemistry such as the widely used BOC (t-butyloxycarbonyl) group allows the chemistry to be directed specifically towards the substitution pattern shown. Other approaches such as via the chemistry of ethyleneimine may also lead to a subset of the macrocyclic aza di-X series. In order to obtain the un-symmetrically substituted derivatives a variant of some chemistry above could be used:

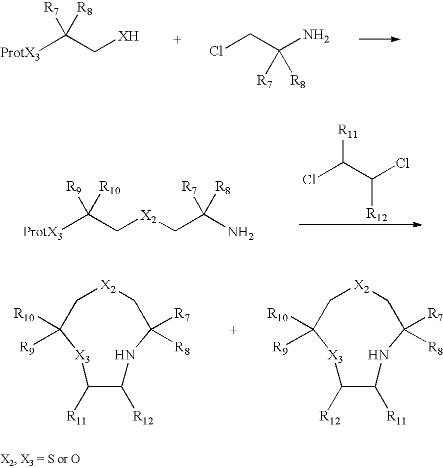

The incorporation of R₁ and R₂ can by accomplished with this chemistry by standard procedures.

Copper antagonists and pharmaceutically acceptable salts for use according to the present invention may also be synthesized using methods described in U.S. Published Patent Application No. 2006/0041170, the contents of which are hereby incorporated by reference in its entirety.

Any of the methods of treating a subject having or suspected of having or predisposed to, or at risk for, a disease, disorder, and/or condition, referenced or described herein may utilize the administration of any of the doses, dosage forms, formulations, compositions and/or devices herein described.

Aspects of the invention include controlled or other doses, dosage forms, formulations, compositions and/or devices containing one or more 3-hydroxy-3-methylglutaryl coenzyme A reductase inhibitors and one or more copper antagonists, for example, one or more compounds of Formulae I, I(a) or II, or trientine active agents, including but not limited to, trientine, trientine dihydrochloride, trientine disuccinate, trientine tetramaleate, trientine tetraflimarate or other pharmaceutically acceptable salts thereof, trientine analogues of Formulae I, I(a) and II and salts thereof. The present invention includes, for example, doses and dosage forms for at least oral administration, transdermal delivery, topical application, suppository delivery, transmucosal delivery, injection (including subcutaneous administration, subdermal administration, intramuscular administration, depot administration, and intravenous administration (including delivery via bolus, slow intravenous injection, and intravenous drip), infusion devices (including implantable infusion devices, both active and passive), administration by inhalation or insufflation, buccal administration, sublingual administration, and ophthalmic administration.

The invention includes, for example, methods for treating a subject having or suspected of having or predisposed to, or at risk for, any diseases, disorders and/or conditions characterized in whole or in part by (a) hypercupremia and/or copper-related tissue damage and (b) hyperlipidemia, hypercholesterolemia (e.g., elevated cholesterol in low-density lipoprotein (LDL-C)), hypertension, hyperglycemia, insulin resistance, impaired glucose tolerance, and/or impaired fasting glucose, comprising administering a composition comprising a pharmaceutically acceptable copper antagonist and a 3-hydroxy-3-methylglutaryl coenzyme A reductase inhibitor. Such compounds may be administered in amounts, for example, that are effective to (1) decrease body and/or tissue copper levels, (2) increase copper output in the urine of a subject, (3) decrease copper uptake, for example, in the gastrointestinal tract, 4) decrease SOD, for example, EC-SOD, as measured by mass or activity, (5) decrease homocysteine, (6) decrease oxidative stress (7) increase copper (I), and/or (8) lower LDL-C.

The invention includes methods for treating and/or preventing, in whole or in part, various diseases, disorders and conditions, including, for example, atherosclerosis, coronary heart disease, hypercholesterolemia, hyperlipidemia, hypertension, impaired glucose tolerance; impaired fasting glucose; diabetes and/or its complications, including type 1 and type 2 diabetes and their complications; insulin resistance; Syndrome X; obesity and other weight related disorders; cardiomyopathy, including diabetic cardiomyopathy; hyperglycemia, and/or hyperinsulinemia, tissue ischemia, and diseases and disorders characterized at least in part by any one or more of hyperglycemia, hypercholesterolemia, hypertension, hyperinsulinemia, hyperlipidemia, atherosclerosis, and tissue ischemia; and, diseases, disorders or conditions characterized in whole or in part by (a) hypercupremia and/or copper-related tissue damage and (b) elevated or undesired LDL-C levels, hypercholesterolemia, hypertension, hyperglycemia, insulin resistance, impaired glucose tolerance, and/or impaired fasting glucose, or predisposition to, or risk for, (a) and (b). Other disorders to be treated using the compositions and methods of the invention include disorders of the heart muscle, including heart failure; myocardial infarction; cardiomyopathy, including idiopathic cardiomyopathy, metabolic cardiomyopathy, alcoholic cardiomyopathy, drug-induced cardiomyopathy, ischemic cardiomyopathy, and hypertensive cardiomyopathy. Still other disorders that may be treated using the compositions and methods of the invention are hypertension and stroke. Other disorders that may be treated using the compositions and methods of the invention include diabetic acute coronary syndrome (e.g., myocardial infarction, diabetic hypertensive cardiomyopathy, acute coronary syndrome associated with impaired glucose tolerance (IGT), acute coronary syndrome associated with impaired fasting glucose (IFG), hypertensive cardiomyopathy associated with IGT, hypertensive cardiomyopathy associated with IFG, ischemic cardiomyopathy associated with IGT, ischemic cardiomyopathy associated with IFG, ischemic cardiomyopathy associated with coronary heart disease (CHD), acute coronary syndrome not associated with any abnormality of the glucose metabolism, hypertensive cardiomyopathy not associated with any apparent abnormality of glucose metabolism, ischemic cardiomyopathy not associated with any apparent abnormality of glucose metabolism (irrespective of whether or not such ischemic cardiomyopathy is associated with coronary heart disease or not), and any disease of the vascular tree including disease states of the aorta, carotid, cerebrovascular, coronary, renal, retinal, vasa nervorum, iliac, femoral, popliteal, arteriolar tree and capillary bed. Additionally, atheromatous disorders of the major blood vessels (including the aorta, the coronary arteries, the carotid arteries, the cerebrovascular arteries, the renal arteries, the iliac arteries, the femoral arteries, and the popliteal arteries), toxic, drug-induced, and metabolic disorders of small blood vessels, and, non-fatal plaque rupture of atheromatous lesions of major blood vessels, all may be treated using the compositions and methods of the invention.

A therapeutically effective amount of a copper antagonist, for example a copper chelator, including but not limited to trientine, trientine salts, trientine analogues of Formulae I, I(a) and II, and so on, is from about 1 mg/kg to about 1 g/kg. Other therapeutically effective dose ranges include, for example, from about 1.5 mg/kg to about 950 mg/kg, about 2 mg/kg to about 900 mg/kg, about 3 mg/kg to about 850 mg/kg, about 4 mg/kg to about 800 mg/kg, about 5 mg/kg to about 750 mg/kg, about 5 mg/kg to about 700 mg/kg, about 5 mg/kg to about 600 mg/kg, about 5 mg/kg to about 500 mg/kg, about 10 mg/kg to about 400 mg/kg, about 10 mg/kg to about 300 mg/kg, about 10 mg/kg to about 200 mg/kg, about 10 mg/kg to about 250 mg/kg, about 10 mg/kg to about 200 mg/kg, about 10 mg/kg to about 200 mg/kg, about 10 mg/kg to about 150 mg/kg, about 10 mg/kg to about 100 mg/kg, about 10 mg/kg to about 75 mg/kg, about 10 mg/kg to about 50 mg/kg, or about 15 mg/kg to about 35 mg/kg.

In some embodiments of the invention, a therapeutically effective amount of a copper. antagonist, including for example, trientine, trientine salts, trientine analogues of Formulae I, I(a) and II, and so on, is from about 10 mg to about 4 g per day. Other therapeutically effective dose ranges include, for example, from about 20 mg to about 3.9 g, from about 30 mg to about 3.7 g, from about 40 mg to about 3.5 g, from about 50 mg to about 3 g, from about 60 mg to about 2.8 g, from about 70 mg to about 2.5 g, about 80 mg to about 2.3 g, about 100 mg to about 2 g, about 100 mg to about 1.5 g, about 200 mg to about 1400 mg, about 200 mg to about 1300 mg, about 200 mg to about 1200 mg, about 200 mg to about 1100 mg, about 200 mg to about 1000 mg, about 300 mg to about 900 mg, about 300 mg to about 800 mg, about 300 mg to about 700 mg or about 300 mg to about 600 mg per day.

Copper antagonists including, for example, trientine, trientine salts, trientine analogues of Formulae I, I(a) and II, and so on, will also be effective at doses in the order of 1/10, 1/50, 1/100, 1/200, 1/300, 1/400, 1/500 and even 1/1000 of those described herein.

The invention accordingly, in part, provides low dose compositions, formulations and devices comprising one or more copper antagonists. For example, low dose copper antagonists may include compounds, including copper chelators, particularly Cu+2 chelators, including but not limited to trientine active agents and compounds of Formulae I, I(a) and II, and the like, in an amount sufficient to provide, for example, dosages from about 0.001 mg/kg to about 5 mg/kg, about 0.01 mg/kg to about 4.5 mg/kg, about 0.02 mg/kg to about 4 mg/kg, about 0.02 to about 3.5 mg/kg, about 0.02 mg/kg to about 3 mg/kg, about 0.05 mg/kg to about 2.5 mg/kg, about 0.05 mg/kg to about 2 mg/kg, about 0.05-0.1 mg/kg to about 5 mg/kg, about 0.05-0.1 mg/kg to about 4 mg/kg, about 0.05-0.1 mg/kg to about 3 mg/kg, about 0.05-0.1 mg/kg to about 2 mg/kg, about 0.05-0.1 mg/kg to about 1 mg/kg, and/or any other doses or dose ranges within the ranges set forth herein.

In some embodiments of the invention, a therapeutically effective amount is an amount effective to elicit a plasma concentration of a copper antagonist, for example, a copper chelator, including for example, trientine active agents, including but not limited to trientine, trientine salts, and compounds of Formulae I, I(a) and II, and so on, from about 0.01 mg/L to about 20 mg/L, about 0.01 mg/L to about 15 mg/L, about 0.1 mg/L to about 10 mg/L, about 0.5 mg/L to about 9 mg/L, about 1 mg/L to about 8 mg/L, about 2 mg/L to about 7mg/L or about 3 mg/L to about 6 mg/L.

Dose ranges for 3-hydroxy-3-methylglutaryl coenzyme A reductase inhibitors are discussed herein and additionally are known to those skilled in the art.

The doses described herein, may be administered in a single dose or multiple doses. For example, doses may be administered, once, twice, three, four or more times a day.

Any such dose may be administered by any of the routes or in any of the forms herein described. It will be appreciated that any of the dosage forms, compositions, formulations or devices described herein particularly for oral administration may be utilized, where applicable or desirable, in a dosage form, composition, formulation or device for administration by any of the other routes herein contemplated or commonly employed. For example, a dose or doses could be given parenterally using a dosage form suitable for parenteral administration which may incorporate features or compositions described in respect of dosage forms suitable for oral administration, or be delivered in an oral dosage form such as a modified release, extended release, delayed release, slow release or repeat action oral dosage form.

Thus, the invention also is directed to doses, dosage forms, formulations, compositions and/or devices comprising one or more 3-hydroxy-3-methylglutaryl coenzyme A reductase inhibitors and one or more copper antagonists, for example, one or more compounds of Formulae I, I(a) and II and salts thereof, and one or more trientine active agents, including but not limited to, trientine, trientine dihydrochloride, trientine disuccinate, trientine tetramaleate, trientine tetrafumarate or other pharmaceutically acceptable salts thereof, trientine analogues and salts thereof, useful for therapy of diseases, disorders, and/or conditions in humans and other mammals and other disorders as disclosed herein. The use of these dosage forms, formulations compositions and/or devices of copper antagonism enables effective treatment of these conditions, through novel and improved formulations suitable for administration to humans and other mammals.

The invention provides, for example, dosage forms, formulations, devices and/or compositions containing one or more 3-hydroxy-3-methylglutaryl coenzyme A reductase inhibitors and one or more copper antagonists, for example, copper chelators, such as copper (II) chelators), including one or more compounds of Formulae I, I(a) and II and salts thereof, and trientine active agents, including but not limited to, trientine, trientine dihydrochloride, trientine disuccinate, trientine tetramaleate, trientine tetrafumarate or other pharmaceutically acceptable salts thereof, and salts thereof. The dosage forms, formulations, devices and/or compositions of the invention may be formulated to optimize bioavailability and to maintain plasma concentrations within therapeutic range, including for extended periods, and results in increases in the time that plasma concentrations of the 3-hydroxy-3-methylglutaryl coenzyme A reductase inhibitor(s) and/or copper antagonist(s) remain within a desired therapeutic range at the site or sites of action. Controlled delivery preparations also optimize the drug concentration at the site of action and minimize periods of under and over medication, for example.

The dosage forms, formulated, devices and/or compositions of the invention may be formulated for periodic administration, including once daily administration, to provide low dose controlled and/or low dose long-lasting in vivo release of a 3-hydroxy-3-methylglutaryl coenzyme A reductase inhibitor and a copper antagonist, for example, a copper chelator for chelation of copper and excretion of copper via the urine and/or to provide enhanced bioavailability of a 3-hydroxy-3-methylglutaryl coenzyme A reductase inhibitor/copper antagonist, such as a copper chelator for chelation of copper and excretion of copper via the urine.

Examples of dosage forms suitable for oral administration include, but are not limited to tablets, capsules, lozenges, or like forms, or any liquid forms such as syrups, aqueous solutions, emulsions and the like, capable of providing a therapeutically effective amount of a 3-hydroxy-3-methylglutaryl coenzyme A reductase inhibitor/copper antagonist.

Examples of dosage forms suitable for transdermal administration include, but are not limited, to transdermal patches, transdermal bandages, and the like. Examples of dosage forms suitable for topical administration of the compounds and formulations of the invention are any lotion, stick, spray, ointment, paste, cream, gel, etc. whether applied directly to the skin or via an intermediary such as a pad, patch or the like.

Examples of dosage forms suitable for suppository administration of the compounds and formulations of the invention include any solid dosage form inserted into a bodily orifice particularly those inserted rectally, vaginally and urethrally.

Examples of dosage forms suitable for transmucosal delivery of the compounds and formulations of the invention include depositories solutions for enemas, pessaries, tampons, creams, gels, pastes, foams, nebulised solutions, powders and similar formulations containing in addition to the active ingredients such carriers as are known in the art to be appropriate.

Examples of dosage of forms suitable for injection of the compounds and formulations of the invention include delivery via bolus such as single or multiple administrations by intravenous injection, subcutaneous, subdermal, and intramuscular administration or oral administration.

Examples of dosage forms suitable for depot administration of the compounds and formulations of the invention include pellets or small cylinders of active agent or solid forms wherein the active agent is entrapped in a matrix of biodegradable polymers, microemulsions, liposomes or is microencapsulated.

Examples of infusion devices for compounds and formulations of the invention include infusion pumps containing one or more 3-hydroxy-3-methylglutaryl coenzyme A reductase inhibitors and one or more copper antagonists, for example one or more copper chelators, such as for example, one or more compounds of Formulae I, I(a) and II and salts thereof, or trientine active agents, including but not limited to, trientine, trientine dihydrochloride, trientine disuccinate, trientine tetramaleate, trientine tetrafumarate or other pharmaceutically acceptable salts thereof, at a desired amount for a desired number of doses or steady state administration, and include implantable drug pumps.

Examples of implantable infusion devices for compounds, and formulations of the invention include any solid form in which the active agent is encapsulated within or dispersed throughout a biodegradable polymer or synthetic, polymer such as silicone, silicone rubber, silastic or similar polymer.

Examples of dosage forms suitable for inhalation or insufflation of the compounds and formulations of the invention include compositions comprising solutions and/or suspensions in pharmaceutically acceptable, aqueous, or organic solvents, or mixture thereof and/or powders.

Examples of dosage forms suitable for buccal administration of the compounds and formulations of the invention include lozenges, tablets and the like, compositions comprising solutions and/or suspensions in pharmaceutically acceptable, aqueous, or organic solvents, or mixtures thereof and/or powders.

Examples of dosage forms suitable for sublingual administration of the compounds and formulations of the invention include lozenges, tablets and the like, compositions comprising solutions and/or suspensions in pharmaceutically acceptable, aqueous, or organic solvents, or mixtures thereof and/or powders.

Examples of dosage forms suitable for opthalmic administration of the compounds and formulations of the invention include inserts and/or compositions comprising solutions and/or suspensions in pharmaceutically acceptable, aqueous, or organic solvents.

Examples of controlled drug formulations useful for delivery of the compounds and formulations of the invention are found in, for example, Sweetman, S. C. (Ed.). Martindale. The Complete Drug Reference, 33rd Edition, Pharmaceutical Press, Chicago, 2002, 2483 pp.; Aulton, M. E. (Ed.) Pharmaceutics. The Science of Dosage Form Design. Churchill Livingstone, Edinburgh, 2000, 734 pp.; and, Ansel, H. C., Allen, L. V. and Popovich, N. G. Pharmaceutical Dosage Forms and Drug Delivery Systems, 7th Ed., Lippincott 1999, 676 pp. Excipients employed in the manufacture of drug delivery systems are described in various publications known to those skilled in the art including, for example, Kibbe, E. H. Handbook of Pharmaceutical Excipients, 3rd Ed., American Pharmaceutical Association, Washington, 2000, 665 pp. The USP also provides examples of modified-release oral dosage forms, including those formulated as tablets or capsules. See, for example, The United States Pharmacopeia 23/National Formulary 18, The United States Pharmacopeial Convention, Inc., Rockville Md., 1995 (hereinafter “the USP”), which also describes specific tests to determine the drug release capabilities of extended-release and delayed-release tablets and capsules. The USP test for drug release for extended-release and delayed-release articles is based on drug dissolution from the dosage unit against elapsed test time. Descriptions of various test apparatus and procedures may be found in the USP. The individual monographs contain specific criteria for compliance with the test and the apparatus and test procedures to be used. Examples have been given, for example for the release of aspirin from Aspirin Extended-release Tablets (for example, see: Ansel, H. C. Allen, L. V. and Popovich, N. G., Pharmaceutical Dosage Forms and Drug Delivery Systems, 7th Ed., Lippincott 1999, p. 237). Modified-release tablets and capsules must meet the USP standard for uniformity as described for conventional dosage units. Uniformity of dosage units may be demonstrated by either of two methods, weight variation or content uniformity, as described in the USP. Further guidance concerning the analysis of extended release dosage forms has been provided by the F.D.A. (See Guidance for Industry. Extended release oral dosage forms: development, evaluation, and application of in vitro/in vivo correlations. Rockville, Md.: Center for Drug Evaluation and Research, Food and Drug Administration, 1997).

Further examples of dosage forms of the invention include, but are not limited to modified-release (MR) dosage forms including delayed-release (DR) forms; prolonged-action (PA) forms; controlled-release (CR) forms; extended-release (ER) forms; timed-release (TR) forms; and long-acting (LA) forms. For the most part, these terms are used to describe orally administered dosage forms, however these terms may be applicable to any of the dosage forms, formulations, compositions and/or devices described herein. These formulations effect delayed total drug release for some time after drug administration, and/or drug release in small aliquots intermittently after administration, and/or drug release slowly at a controlled rate governed by the delivery system, and/or drug release at a constant rate that does not vary, and/or drug release for a significantly longer period than usual formulations.

Modified-release dosage forms of the invention include dosage forms having drug release features based on time, course, and/or location which are designed to accomplish therapeutic or convenience objectives not offered by conventional or immediate-release forms. See, for example, Bogner, R. H. Bioavailability and bioequivalence of extended-release oral dosage forms. U.S. Pharmacist 22 (Suppl.):3-12 (1997); Scale-up of oral extended-release drug delivery systems: part I, an overview. Pharmaceutical Manufacturing 2:23-27 (1985). Extended-release dosage forms of the invention include, for example, as defined by The United States Food and Drug Administration (FDA), a dosage form that allows a reduction in dosing frequency to that presented by a conventional dosage form, e.g., a solution or an immediate-release dosage form. See, for example, Bogner, R. H. Bioavailability and bioequivalence of extended-release oral dosage forms. US Pharmacist 22 (Suppl.):3-12 (1997); Guidance for industry. Extended release oral dosage forms: development, evaluation, and application of the in vitro/in vivo correlations. Rockville, Md.: Center for Drug Evaluation and Research, Food and Drug Administration (1997). Repeat action dosage forms of the invention include, for example, forms that contain two single doses of medication, one for immediate release and the second for delayed release. Bi-layered tablets, for example, may be prepared with one layer of drug for immediate release with the second layer designed to release drug later as either a second dose or in an extended-release manner. Targeted-release dosage forms of the invention include, for example, formulations that facilitate drug release and which are directed towards isolating or concentrating a drug in a body region, tissue, or site for absorption or for drug action.

The invention in part provides dosage forms, formulations, devices and/or compositions and/or methods utilizing administration of dosage forms, formulations, devices and/or compositions incorporating one or more 3-hydroxy-3-methylglutaryl coenzyme A reductase inhibitors and one or more copper antagonists, for example one or more copper chelators, such as for example, one or more compounds of Formulae I, I(a) or II and salts thereof, and trientine active agents, including but not limited to, trientine, trientine dihydrochloride, trientine disuccinate, trientine tetramaleate, trientine tetrafumarate or other pharmaceutically acceptable salts thereof, complexed with one or more suitable anions to yield complexes that are only slowly soluble in body fluids. One such example of modified release forms of one or more 3-hydroxy-3-methylglutaryl coenzyme A reductase inhibitors and/or one or more copper antagonists is produced by the incorporation of the active agent or agents into certain complexes such as those formed with the anions of various forms of tannic acid (for example, see: Merck Index 12th Ed., 9221). Dissolution of such complexes may depend, for example, on the pH of the environment. This slow dissolution rate provides for the extended release of the 3-hydroxy-3-methylglutaryl coenzyme A reductase inhibitor/copper antagonist. For example, salts of tannic acid, and/or tannates, provide for this quality, and are expected to possess utility for the treatment of conditions in which increased copper plays a role. Examples of equivalent products are provided by those having the tradename Rynatan (Wallace: see, for example, Madan, P. L., “Sustained release dosage forms,” U.S. Pharmacist 15:39-50 (1990); Ryna-12 S, which contains a mixture of mepyramine tannate with phenylephrine tannate, Martindale 33rd Ed., 2080.4).

Also included in the invention are coated beads, granules or microspheres containing one or more 3-hydroxy-3-methylglutaryl coenzyme A reductase inhibitors and one or more copper antagonists. Thus, the invention also provides a method to achieve modified release of one or more 3-hydroxy-3-methylglutaryl coenzyme A reductase inhibitors and one or more copper antagonists by incorporation of the drug into coated beads, granules, or microspheres. Such formulations of one or more 3-hydroxy-3-methylglutaryl coenzyme A reductase inhibitors and one or more copper antagonists have utility for the treatment of diseases in humans and other mammals in which a 3-hydroxy-3-methylglutaryl coenzyme A reductase inhibitor/copper antagonist, for example, trientine, is indicated. In such systems, the 3-hydroxy-3-methylglutaryl coenzyme A reductase inhibitor and/or copper antagonist is distributed onto beads, pellets, granules or other particulate systems. Using conventional pan-coating or air-suspension coating techniques, a solution of the 3-hydroxy-3-methylglutaryl coenzyme A reductase inhibitor/copper antagonist substance is placed onto small inert nonpareil seeds or beads made of sugar and starch or onto microcrystalline cellulose spheres. The nonpareil seeds are most often in the 425 to 850 micrometer range whereas the microcrystalline cellulose spheres are available ranging from 170 to 600 micrometers (see Ansel, H. C., Allen, L. V. and Popovich, N. G., Pharmaceutical Dosage Forms and Drug Delivery Systems, 7th Ed., Lippincott 1999, p. 232). The microcrystalline spheres are considered more durable during production than sugar-based cores (see: Celphere microcrystalline cellulose spheres. Philadelphia: FMC Corporation, 1996). Methods for manufacture of microspheres suitable for drug delivery have been described (see, for example, Arshady, R. Microspheres and microcapsules: a survey of manufacturing techniques. 1: suspension and cross-linking. Polymer Eng Sci 30:1746-1758 (1989); see also, Arshady, R., Micro-spheres and microcapsules: a survey of manufacturing techniques. 2: coacervation. Polymer Eng Sci 30:905-914 (1990); see also: Arshady R., Microspheres and microcapsules: a survey of manufacturing techniques. 3: solvent evaporation. Polymer Eng Sci 30:915-924 (1990). In instances in which the 3-hydroxy-3-methylglutaryl coenzyme A reductase inhibitor and/or the copper antagonist dose is large, the starting granules of material may be composed of the 3-hydroxy-3-methylglutaryl coenzyme A reductase inhibitor and/or the copper antagonist itself. Some of these granules may remain uncoated to provide immediate 3-hydroxy-3-methylglutaryl coenzyme A reductase inhibitor/copper antagonist release. Other granules (about two-thirds to three-quarters) receive varying coats of a lipid material such as beeswax, camauba wax, glycerylmonostearate, cetyl alcohol, or a cellulose material such as ethylcellulose (infra). Subsequently, granules of different coating thickness are blended to achieve a mixture having the desired release characteristics. The coating material may be coloured with one or more dyes to distinguish granules or beads of different coating thickness (by depth of colour) and to provide distinctiveness to the product. When properly blended, the granules may be placed in capsules or tablets. Various coating systems are commercially available which are aqueous-based and which use ethylcellulose and plasticizer as the coating material (e.g., Aquacoat™ [FMC Corporation, Philadelphia] and Surerelease™ [Colorcon]; Aquacoat aqueous polymeric dispersion. Philadelphia: FMC Corporation, 1991; Surerelease aqueous controlled release coating system. West Point, Pa.: Colorcon, 1990; Butler, J., Cumming, I, Brown, J. et al., A novel multiunit controlled-release system, Pharm Tech 22:122-138 (1998); Yazici, E., Oner, L., Kas, H. S. & Hincal, A. A., Phenytoin sodium microspheres: bench scale formulation, process characterization and release kinetics, Pharmaceut Dev Technol 1:175-183 (1996)). Aqueous-based coating systems eliminate the hazards and environmental concerns associated with organic solvent-based systems. Aqueous and organic solvent-based coating methods have been compared (see, for example, Hogan, J. E. Aqueous versus organic solvent coating. Int J Pharm Tech Prod Manufacture 3:17-20 (1982)). The variation in the thickness of the coats and in the type of coating materials used affects the rate at which the body fluids are capable of penetrating the coating to dissolve the 3-hydroxy-3-methylglutaryl coenzyme A reductase inhibitor/copper antagonist. Generally, the thicker the coat, the more resistant to penetration and the more delayed will be 3-hydroxy-3-methylglutaryl coenzyme A reductase inhibitor/copper antagonist release and dissolution. Typically, the coated beads are about 1 mm in diameter. They are usually combined to have three or four release groups among the more than 100 beads contained in the dosing unit (see Madan, P. L. Sustained release dosage forms. U.S. Pharmacist 15:39-50 (1990)). This provides the different desired sustained or extended release rates and the targeting of the coated beads to the desired segments of the gastrointestinal tract. One example of this type of dosage form is the Spansule™ (SmithKline Beecham Corporation, U.K.). Examples of film-forming polymers which can be used in water-insoluble release-slowing intermediate layer(s) (to be applied to a pellet, spheroid or tablet core) include ethylcellulose, polyvinyl acetate, Eudragit® RS, Eudragit® RL, etc. (Each of Eudragit® RS and Eudragit® RL is an ammonio methacrylate copolymer. The release rate can be controlled not only by incorporating therein suitable water-soluble pore formers, such as lactose, mannitol, sorbitol, etc., but also by the thickness of the coating layer applied. Multi tablets may be formulated which include small spheroid-shaped compressed minitablets that may have a diameter of between 3 to 4 mm and can be placed in gelatin capsule shell to provide the desired pattern of 3-hydroxy-3-methylglutaryl coenzyme A reductase inhibitor/copper antagonist release. -Each capsule may contain 8-10 minitablets, some uncoated for immediate release and others coated for extended release of the 3-hydroxy-3-methylglutaryl coenzyme A reductase inhibitor/copper antagonist.

A number of methods may be employed to generate modified-release dosage forms of one or more 3-hydroxy-3-methylglutaryl coenzyme A reductase inhibitors and one or more copper antagonists suitable for oral administration to humans and other mammals. Two basic mechanisms are available to achieve modified release drug delivery. These are altered dissolution or diffusion of drugs and excipients. Within this context, for example, four processes may be employed, either simultaneously or consecutively. These are as follows: (i) hydration of the device (e.g., swelling of the matrix); (ii) diffusion of water into the device; (iii) controlled or delayed dissolution of the drug; and (iv) controlled or delayed diffusion of dissolved or solubilized drug out of the device. See, e.g., Examples 11, 12, 23, 24, 35, and 36 herein.

For orally administered dosage forms of the compounds and formulations of the invention, extended 3-hydroxy-3-methylglutaryl coenzyme A reductase inhibitor and/or copper antagonist action, for example, copper chelator action, may be achieved by affecting the rate at which the 3-hydroxy-3-methylglutaryl coenzyme A reductase inhibitor and/or copper antagonist is released from the dosage form and/or by slowing the transit time of the dosage form through the gastrointestinal tract (see Bogner, R. H., Bioavailability and bioequivalence of extended-release oral dosage forms. US Pharmacist 22 (Suppl.):3-12 (1997)). The rate of drug release from solid dosage forms may be modified by the technologies described below which, in general, are based on the following: 1) modifying drug dissolution by controlling access of biologic fluids to the drug through the use of barrier coatings; 2) controlling drug diffusion rates from dosage forms; and 3) chemically reacting or interacting between the drug substance or its pharmaceutical barrier and site-specific biological fluids. Systems by which these objectives are achieved are also provided herein. In one approach, employing digestion as the release mechanism, the 3-hydroxy-3-methylglutaryl coenzyme A reductase inhibitor/copper antagonist is either coated or entrapped in a substance that is slowly digested or dispersed into the intestinal tract. The rate of availability of the 3-hydroxy-3-methylglutaryl coenzyme A reductase inhibitor/copper antagonist is a function of the rate of digestion of the dispersible material. Therefore, the release rate, and thus the effectiveness of the 3-hydroxy-3-methylglutaryl coenzyme A reductase inhibitor/copper antagonist, varies from subject to subject depending upon the ability of the subject to digest the material.

A further form of slow release dosage form of the compounds and formulations of the invention is any suitable osmotic system where semipermeable membranes of for example cellulose acetate, cellulose acetate butyrate, cellulose acetate propionate, is used to control the release of 3-hydroxy-3-methylglutaryl coenzyme A reductase inhibitor/copper antagonist. These can be coated with aqueous dispersions of enteric lacquers without changing release rate. An example of such an osmotic system is an osmotic pump device, an example of which is the Oros™ device developed by Alza Inc. (U.S.A.). This system comprises a core tablet surrounded by a semi-permeable membrane coating having a 0.4 mm diameter hole produced by a laser beam. The core tablet has two layers, one containing the drug (the “active” layer) and the other containing a polymeric osmotic agent (the “push” layer). The core layer consists of active drug, filler, a viscosity modulator, and a solubilizer. The system operates on the principle of osmotic pressure. This system is suitable for delivery of a wide range of 3-hydroxy-3-methylglutaryl coenzyme A reductase inhibitors and copper antagonists, including the compounds of Formulae I, I(a) and II, and trientine active agents, or salts of any of them. The coating technology is straightforward, and release is zero-order. When the tablet is swallowed, the semi-permeable membrane permits aqueous fluid to enter from the stomach into the core tablet, dissolving or suspending the 3-hydroxy-3-methylglutaryl coenzyme A reductase inhibitor/copper antagonist. As pressure increases in the osmotic layer, it forces or pumps the 3-hydroxy-3-methylglutaryl coenzyme A reductase inhibitors/copper antagonist solution out of the delivery orifice on the side of the tablet. Only the 3-hydroxy-3-methylglutaryl coenzyme A reductase inhibitor/copper antagonist solution (not the undissolved 3-hydroxy-3-methylglutaryl coenzyme A reductase inhibitor/copper antagonist) is capable of passing through the hole in the tablet. The system is designed such that only a few drops of water are drawn into the tablet each hour. The rate of inflow of aqueous fluid and the function of the tablet depends on the existence of an osmotic gradient between the contents of the bi-layer and the fluid in the gastrointestinal tract. Delivery is essentially constant as long as the osmotic gradient remains unchanged. The 3-hydroxy-3-methylglutaryl coenzyme A reductase inhibitor/copper antagonist release rate may be altered by changing the surface area, the thickness or composition of the membrane, and/or by changing the diameter of the 3-hydroxy-3-methylglutaryl coenzyme A reductase inhibitor/copper antagonist release orifice. The 3-hydroxy-3-methylglutaryl coenzyme A reductase inhibitor/copper antagonist release rate is not affected by gastrointestinal acidity, alkalinity, fed conditions, or gut motility. The biologically inert components of the tablet remain intact during gut transit and are eliminated in the feces as an insoluble shell. Other examples of the application of this technology are provided by Glucotrol XL Extended Release Tablets (Pfizer Inc.) and Procardia XL Extended Release Tablets (Pfizer Inc.; see, Martindale 33rd Ed., p. 2051.3).

The invention also provides devices for compounds and formulations of the invention that utilize monolithic matrices including, for example, slowly eroding or hydrophilic polymer matrices, in which one or more 3-hydroxy-3-methylglutaryl coenzyme A reductase inhibitors/copper antagonists is/are compressed or embedded.

Monolithic matrix devices comprising compounds and formulations of the invention include those formed using either of the following systems, for example: (I), 3-hydroxy-3-methylglutaryl coenzyme A reductase inhibitor/copper antagonist dispersed in a soluble matrix, which become increasingly available as the matrix dissolves or swells; examples include hydrophilic colloid matrices, such as hydroxypropylcellulose (BP) or hydroxypropyl cellulose (USP); hydroxypropyl methylcellulose (HPMC; BP, USP); methylcellulose (MC; BP, USP); calcium carboxymethylcellulose (Calcium CMC; BP, USP); acrylic acid polymer or carboxy polymethylene (Carbopol) or Carbomer (BP, USP); or linear glycuronan polymers such as alginic acid (BP, USP), for example those formulated into microparticles from alginic acid (alginate)-gelatin hydrocolloid coacervate systems, or those in which liposomes have been encapsulated by coatings of alginic acid with poly-L-lysine membranes. 3-hydroxy-3-methylglutaryl coenzyme A reductase inhibitor/copper antagonist release occurs as the polymer swells, forming a matrix layer that controls the diffusion of aqueous fluid into the core and thus the rate of diffusion of 3-hydroxy-3-methylglutaryl coenzyme A reductase inhibitor/copper antagonist from the system. In such systems, the rate of 3-hydroxy-3-methylglutaryl coenzyme A reductase inhibitor/copper antagonist release depends upon the tortuous nature of the channels within the gel, and the viscosity of the entrapped fluid, such that different release kinetics can be achieved, for example, zero-order, or first-order combined with pulsatile release. Where such gels are not cross-linked, there is a weaker, non-permanent association between the polymer chains, which relies on secondary bonding. With such devices, high loading of the 3-hydroxy-3-methylglutaryl coenzyme A reductase inhibitor/copper antagonist is achievable, and effective blending is frequent. Devices may contain 20-80% of 3-hydroxy-3-methylglutaryl coenzyme A reductase inhibitor/copper antagonist (w/w), along with gel modifiers that can enhance 3-hydroxy-3-methylglutaryl coenzyme A reductase inhibitor/copper antagonist diffusion; examples of such modifiers include sugars that can enhance the rate of hydration, ions that can influence the content of cross-links, and pH buffers that affect the level of polymer ionization. Hydrophilic matrix devices of the invention may also contain one or more of pH buffers, surfactants, counter-ions, lubricants such as magnesium stearate (BP, USP) and a glidant such as colloidal silicon dioxide (USP; colloidal anhydrous silica, BP) in addition to 3-hydroxy-3-methylglutaryl coenzyme A reductase inhibitor/copper antagonist and hydrophilic matrix; (II) 3-hydroxy-3-methylglutaryl coenzyme A reductase inhibitor/copper antagonist particles are dissolved in an insoluble matrix, from which 3-hydroxy-3-methylglutaryl coenzyme A reductase inhibitor/copper antagonist becomes available as solvent enters the matrix, often through channels, and dissolves the 3-hydroxy-3-methylglutaryl coenzyme A reductase inhibitor/copper antagonist particles. Examples include systems formed with a lipid matrix, or insoluble polymer matrix, including preparations formed from Camauba wax (BP; USP); medium-chain triglyceride such as fractionated coconut oil (BP) or triglycerida saturata media (PhEur); or cellulose ethyl ether or ethylcellulose (BP, USP). Lipid matrices are simple and easy to manufacture, and incorporate the following blend- of powdered components: lipids (20-40% hydrophobic solids w/w) which remain intact during the release process; 3-hydroxy-3-methylglutaryl coenzyme A reductase inhibitor/copper antagonist, e.g., copper chelator; channeling agent, such as sodium chloride or sugars, which leaches from the formulation, forming aqueous micro-channels (capillaries) through which solvent enters, and through which 3-hydroxy-3-methylglutaryl coenzyme A reductase inhibitor/copper antagonist is released. In the alternative system, which employs an insoluble polymer matrix, the 3-hydroxy-3-methylglutaryl coenzyme A reductase inhibitor/copper antagonist is embedded in an inert insoluble polymer and is released by leaching of aqueous fluid, which diffuses into the core of the device through capillaries formed between particles, and from which 3-hydroxy-3-methylglutaryl coenzyme A reductase inhibitor/copper antagonist diffuses out of the device. The rate of release is controlled by the degree of compression, particle size, and the nature and relative content (w/w) of excipients. An example of such a device is that of Ferrous Gradumet (Martindale 33rd Ed., 1360.3). A further example of a suitable insoluble matrix is an inert plastic matrix. By this method, 3-hydroxy-3-methylglutaryl coenzyme A reductase inhibitor/copper antagonist is granulated with an inert plastic material such as polyethylene, polyvinyl acetate, or polymethacrylate, and the granulated mixture is then compressed into tablets. Once ingested, the 3-hydroxy-3-methylglutaryl coenzyme A reductase inhibitor/copper antagonist is slowly released from the inert plastic matrix by diffusion (see, for example, Bodmeier, R. & Paeratakul, O., “Drug release from laminated polymeric films prepared from aqueous latexes,” J Pharm Sci 79:32-26 (1990); Laghoueg, N., et al., “Oral polymer-drug devices with a core and an erodible shell for constant drug delivery,” Int J Pharm 50:133-139 (1989); Buckton, G., et al., “The influence of surfactants on drug release from acrylic matrices. Int J Pharm 74:153-158 (1991)). The compression of the tablet creates the matrix or plastic form that retains its shape during the leaching of the 3-hydroxy-3-methylglutaryl coenzyme A reductase inhibitor/copper antagonist and through its passage through the gastrointestinal tract. An immediate-release portion of 3-hydroxy-3-methylglutaryl coenzyme A reductase inhibitor/copper antagonist may be compressed onto the surface of the tablet. The inert tablet matrix, expended of 3-hydroxy-3-methylglutaryl coenzyme A reductase inhibitor/copper antagonist, is excreted with the feces. An example of a successful dosage form of this type is Gradumet (Abbott; see, for example, Ferro-Gradumet, Martindale 33rd Ed., p. 1860.4).

Further examples of monolithic matrix devices of the invention have compositions and formulations of the invention incorporated in pendent attachments to a polymer matrix (see, for example, Scholsky, K. M. and Fitch, R.M., Controlled release of pendant bioactive materials from acrylic polymer colloids. J Controlled Release 3:87-108 (1986)). In these devices, 3-hydroxy-3-methylglutaryl coenzyme A reductase inhibitors/copper antagonists, e.g., copper chelators, are attached by means of an ester linkage to poly(acrylate) ester latex particles prepared by aqueous emulsion polymerization.

Yet further examples of monolithic matrix devices of the invention incorporate dosage forms of the compositions and formulations of the invention in which the 3-hydroxy-3-methylglutaryl coenzyme A reductase inhibitor/copper antagonist is/are bound to a biocompatible polymer by a labile chemical bond, e.g., polyanhydrides prepared from a substituted anhydride (itself prepared by reacting an acid chloride with the drug: methacryloyl chloride and the sodium salt of methoxy benzoic acid) have been used to form a matrix with a second polymer (Eudragit RL) which releases drug on hydrolysis in gastric fluid (see: Chafi, N., Montheard, J. P. & Vergnaud, J. M. Release of 2-aminothiazole from polymeric carriers. Int J Pharm 67:265-274 (1992)).

In formulating a successful hydrophilic matrix system for the compositions and formulations of the invention, the polymer selected for use must form a gelatinous layer rapidly enough to protect the inner core of the tablet from disintegrating too rapidly after ingestion. As the proportion of polymer is increased in a formulation so is the viscosity of the gel formed with a resulting decrease in the rate of 3-hydroxy-3-methylglutaryl coenzyme A reductase inhibitor/copper antagonist diffusion and release (see Formulating for controlled release with Methocel Premium cellulose ethers. Midland, MI: Dow Chemical Company, 1995). In general, 20% (w/w) of HPMC results in satisfactory rates of drug release for an extended-release tablet formulation. However, as with all formulations, consideration must be given to the possible effects of other formulation ingredients such as fillers, tablet binders, and disintegrants. An example of a proprietary product formulated using a hydrophilic matrix base of HPMC for extended drug release is that of Oramorph SR Tablets (Roxane; see Martindale 33rd Ed., p. 2014.4).

Two-layered tablets can be manufactured containing one or more of the compositions and formulations of the invention, with one layer containing the uncombined 3-hydroxy-3-methylglutaryl coenzyme A reductase inhibitor and/or copper antagonist for immediate release and the other layer having the 3-hydroxy-3-methylglutaryl coenzyme A reductase inhibitor and/or copper antagonist imbedded in a hydrophilic matrix for extended-release. Three-layered tablets may also be similarly prepared, with both outer layers containing the 3-hydroxy-3-methylglutaryl coenzyme A reductase inhibitor and/or copper antagonist for immediate release. Some commercial tablets are prepared with an inner core containing the extended-release portion of drug and an outer shell enclosing the core and containing drug for immediate release.

The invention also provides forming a complex between the compositions and formulations of the invention and an ion exchange resin, whereupon the complex may be tableted, encapsulated or suspended in an aqueous vehicle. Release of the 3-hydroxy-3-methylglutaryl coenzyme A reductase inhibitor/copper antagonist is dependent on the local pH and electrolyte concentration such that the choice of ion exchange resin may be made so as to preferentially release the 3-hydroxy-3-methylglutaryl coenzyme A reductase inhibitor/copper antagonist in a given region of the alimentary canal. Delivery devices incorporating such a complex are also provided. For example, a modified release dosage form of 3-hydroxy-3-methylglutaryl coenzyme A reductase inhibitor/copper antagonist can be produced by the incorporation of 3-hydroxy-3-methylglutaryl coenzyme A reductase inhibitor/copper antagonist into complexes with an anion-exchange resin. Solutions of 3-hydroxy-3-methylglutaryl coenzyme A reductase inhibitor/copper antagonist may be passed through columns containing an ion-exchange resin to form a complex by the replacement of H₃O⁺ ions. The resin-3-hydroxy-3-methylglutaryl coenzyme A reductase inhibitor/copper antagonist complex is then washed and may be tableted, encapsulated, or suspended in an aqueous vehicle. The release of the 3-hydroxy-3-methylglutaryl coenzyme A reductase inhibitor/copper antagonist is dependent on the pH and the electrolyte concentration in the gastrointestinal fluid. Release is greater in the acidity of the stomach than in the less acidic environment of the small intestine. Alternative examples of this type of extended release preparation are provided by hydrocodone polistirex and chorpheniramine polistirex suspension (Medeva; Tussionex Pennkinetic Extended Release Suspension, see: Martindale 33rd Ed., p. 2145.2) and by phentermine resin capsules (Pharmanex; lonamin Capsules see: Martindale 33rd Ed., p.1916.1). Such resin systems can additionally incorporate polymer barrier coating and bead technologies in addition to the ion-exchange mechanism. The initial dose comes from an uncoated portion, and the remainder from the coated beads, wherein release may be extended over a 12-hour period by ion exchange. The 3-hydroxy-3-methylglutaryl coenzyme A reductase inhibitor and/or copper antagonist containing particles are minute, and may also be suspended to produce a liquid with extended-release characteristics, as well as solid dosage forms. Such preparations may also be suitable for administration, for example in depot preparations suitable for intramuscular injection.

The invention also provides a method to produce modified release preparations of one or more 3-hydroxy-3-methylglutaryl coenzyme A reductase inhibitor/copper antagonists, for example, one or more copper chelators, by microencapsulation. Microencapsulation is a process by which solids, liquids, or even gasses may be encapsulated into microscopic size particles through the formation of thin coatings of “wall” material around the substance being encapsulated such as disclosed in U.S. Pat. Nos. 3,488,418; 3,391,416 and 3,155,590. Gelatin (BP, USP) is commonly employed as a wall-forming material in microencapsulated preparations, but synthetic polymers such as polyvinyl alcohol (USP), ethylcellulose (BP, USP), polyvinyl chloride, and other materials may also be used (see, for example, Zentner, G. M., Rork, G. S., and Himmelstein, K. J., Osmotic flow through controlled porosity films: an approach to delivery of water soluble compounds, J Controlled Release 2:217-229 (1985); Fites, A. L., Banker, G. S., and Smolen, V. F., Controlled drug release through polymeric films, J Pharm Sci 59:610-613 (1970); Samuelov, Y., Donbrow, M., and Friedman, M., Sustained release of drugs from ethylcellulose-polyethylene glycol films and kinetics of drug release, J Pharm Sci 68:325-329 (1979)).

Encapsulation begins with the dissolving of the prospective wall material, say gelatin, in water. One or more 3-hydroxy-3-methylglutaryl coenzyme A reductase inhibitors/copper antagonists, for example, one or more copper chelators, is then added and the two-phase mixture is thoroughly stirred. With the material to be encapsulated broken up to the desired particle size, a solution of a second material is added. This additive material, for example, acacia, is chosen to have the ability to concentrate the gelatin (polymer) into tiny liquid droplets. These droplets (the coacervate) then form a film or coat around the particles of the solid 3-hydroxy-3-methylglutaryl coenzyme A reductase inhibitor/copper antagonist as a consequence of the extremely low interfacial tension of the residual water or solvent in the wall material so that a continuous, tight, film-coating remains on the particle (see Ansel, H. C., Allen, L. V., and Popovich, N. G., Pharmaceutical Dosage Forms and Drug Delivery Systems, 7th Ed., Lippincott 1999, p. 233). The final dry microcapsules are free flowing, discrete particles of coated material. Of the total particle weight, the wall material usually represents between 2 and 20% (w/w). The coated particles are then admixed with tableting excipients and formed into dosage-sized tablets. Different rates of 3-hydroxy-3-methylglutaryl coenzyme A reductase inhibitor/copper antagonist release may be obtained by changing the core-to-wall ratio, the polymer used for the coating, or the method of microencapsulation (for example, see: Yazici, E., Oner, L., Kas, H. S. & Hincal, A. A. Phenytoin sodium microspheres: bench scale formulation, process characterization and release kinetics. Pharmaceut Dev Technol 1996; 1:175-183).

One of the advantages of microencapsulation is that the administered dose of one or more 3-hydroxy-3-methylglutaryl coenzyme A reductase inhibitors/copper antagonists, for example, one or more copper chelators, is subdivided into small units that are spread over a large area of the gastrointestinal tract, which may enhance absorption by diminishing localized 3-hydroxy-3-methylglutaryl coenzyme A reductase inhibitor/copper antagonist concentrations (see Yazici et al., supra). An example of a drug that is commercially available in a microencapsulated extended-release dosage form is potassium chloride (Micro-K Exten-caps, Wyeth-Ayerst, Martindale 33rd Ed., p 1968.1). Other useful approaches include those in which the 3-hydroxy-3-methylglutaryl coenzyme A reductase inhibitor/copper antagonist is incorporated into polymeric colloidal particles or microencapsulates (microparticles, microspheres or nanoparticles) in the form or reservoir and matrix devices (see: Douglas, S. J., et al., “Nanoparticles in drug delivery,” C. R. C. Crit Rev Therap Drug Carrier Syst 3:233-261 (1987); Oppenheim, R. C., “Solid colloidal drug delivery systems: nanoparticles,” Int J Pharm 8:217-234 (1981); Higuchi, T., “Mechanism of sustained action medication: theoretical analysis of rate of release of solid drugs dispersed in solid matrices,” J Pharm Sci 52:1145-1149 (1963)).

The invention also includes repeat action tablets containing one or more 3-hydroxy-3-methylglutaryl coenzyme A reductase inhibitors/copper antagonists, for example, one or more copper chelators. These are prepared so that an initial dose of the 3-hydroxy-3-methylglutaryl coenzyme A reductase inhibitor/copper antagonist is released immediately followed later by a second dose. The tablets may be prepared with the immediate-release dose in the tablet's outer shell or coating with the second dose in the tablet's inner core, separated by a slowly permeable barrier coating. In general, the 3-hydroxy-3-methylglutaryl coenzyme A reductase inhibitor/copper antagonist from the inner core is exposed to body fluids and released 4 to 6 hours after administration. An example of this type of product is proved by Repetabs (Schering Inc.). Repeat action dosage forms are suitable for the administration of one or more 3-hydroxy-3-methylglutaryl coenzyme A reductase inhibitors/copper antagonists for the indications noted herein.

The invention also includes delayed-release oral dosage forms containing one or more 3-hydroxy-3-methylglutaryl coenzyme A reductase inhibitors/copper antagonists, for example, one or more copper chelators. The release of one or more. 3-hydroxy-3-methylglutaryl coenzyme A reductase inhibitors/copper antagonist, for example, one or more copper chelators, from an oral dosage form can be intentionally delayed until it reaches the intestine at least in part by way of, for example, enteric coating. Enteric coatings by themselves are not an efficient method for the delivery of 3-hydroxy-3-methylglutaryl coenzyme A reductase inhibitors/copper antagonists because of the inability of such coating systems to provide or achieve a sustained therapeutic effect after release onset. Enteric coats are designed to dissolve or break down in an alkaline environment. The presence of food may increase the pH of the stomach. Therefore, the concurrent administration of enteric-coated 3-hydroxy-3-methylglutaryl coenzyme A reductase inhibitor/copper antagonists with food or the presence of food in the stomach may lead to dose dumping and unwanted secondary effects. Furthermore, in the event of gastrointestinal side effects, it would be desirable to have a 3-hydroxy-3-methylglutaryl coenzyme A reductase inhibitor/copper antagonist form that is capable of providing the controlled delivery of 3-hydroxy-3-methylglutaryl coenzyme A reductase inhibitors/copper antagonists in a predictable manner over a long period of time. See, e.g., Examples, 11, 12, 23, 24, 35, and 36 herein.

Enteric coatings have application in the present invention when combined or incorporated with one or more of the other dose delivery formulations or devices described herein. This form of delivery conveys the advantage of minimizing the gastric irritation that may be caused in some subjects by 3-hydroxy-3-methylglutaryl coenzyme A reductase inhibitor/copper antagonist such as, for example, trientine. The enteric coating may be time-dependent, pH-dependent where it breaks down in the less acidic environment of the intestine and erodes by moisture over time during gastrointestinal transit, or enzyme-dependent where it deteriorates due to the hydrolysis-catalyzing action of intestinal enzymes (see, for example, Muhammad, N. A., et al., “Modifying the release properties of Eudragit L30D,” Drug Dev Ind Pharm., 17:2497-2509 (1991)). Among the many agents used to enteric coat tablets and capsules known to those skilled in the art are fats including triglycerides, fatty acids, waxes, shellac, and cellulose acetate phthalate although further examples of enteric coated preparations can be found in the USP. See, e.g., Examples 12, 24, and 36 herein.

The invention also provides devices incorporating one or more 3-hydroxy-3-methylglutaryl coenzyme A reductase inhibitors/copper antagonists, for example, one or more copper chelators, in a membrane-control system. Such devices comprise a rate-controlling membrane enclosing a 3-hydroxy-3-methylglutaryl coenzyme A reductase inhibitor/copper antagonist reservoir. Following oral administration the membrane gradually becomes permeable to aqueous fluids, but does not erode or swell. The 3-hydroxy-3-methylglutaryl coenzyme A reductase inhibitor/copper antagonist reservoir may be composed of a conventional tablet, or a microparticle pellet containing multiple units that do not swell following contact with aqueous fluids. The cores dissolve without modifying their internal osmotic pressure, thereby avoiding the risk of membrane rupture, and typically comprise 60:40 mixtures of lactulose: microcrystalline cellulose (w/w). Active drug(s) is/are released through a two-phase process, comprising diffusion of aqueous fluids into the matrix, followed by diffusion of the 3-hydroxy-3-methylglutaryl coenzyme A reductase inhibitor/copper antagonist out of the matrix. Multiple-unit membrane-controlled systems typically comprise more than one discrete unit. They can contain discrete spherical beads individually coated with rate-controlling membrane and may be encapsulated in a hard gelatin shell (examples of such preparations include Contac 400; Martindale 33rd Ed., 1790.1 and Feospan; Martindale 33rd Ed., p.1859.4). Alternatively, multiple-unit membrane-controlled systems may be compressed into a tablet (for example, Suscard; Martindale 33rd Ed., p. 2115.1). Alternative implementations of this technology include devices in which the 3-hydroxy-3-methylglutaryl coenzyme A reductase inhibitor/copper antagonist is coated around inert sugar spheres, and devices prepared by extrusion spheronization employing a conventional matrix system. Advantages of such systems include the more consistent gastro-intestinal transit rate achieved by multiple-unit systems, and the fact that such systems infrequently suffer from catastrophic dose dumping. They are also ideal for the delivery of more than one drug at a time, as disclosed herein.

An example of a sustained release dosage form of one or more compounds and formulations of the invention is a matrix formation, such a matrix formation taking the form of film coated spheroids containing as active ingredient one or more 3-hydroxy-3-methylglutaryl coenzyme A reductase inhibitors/copper antagonists, for example, one or more copper chelators and a non water soluble spheronising agent. The term “spheroid” is known in the pharmaceutical art and means spherical granules having a diameter usually of between 0.01 mm and 4 mm. The spheronising agent may be any pharmaceutically acceptable material that, together with the 3-hydroxy-3-methylglutaryl coenzyme A reductase inhibitor/copper antagonist, can be spheronised to form spheroids. Microcrystalline cellulose is preferred. Suitable microcrystalline cellulose includes, for example, the material sold as Avicel PH 101 (Trade Mark, FMC Corporation). The film-coated spheroids may contain between 70% and 99% (by wt), especially between 80% and 95% (by wt), of the spheronising agent, especially microcrystalline cellulose. In addition to the active ingredient and spheronising agent, the spheroids may also contain a binder. Suitable binders, such as low viscosity, water soluble polymers, will be well known to those skilled in the pharmaceutical art. A suitable binder is, in particular polyvinylpyrrolidone in various degrees of polymerization. However, water-soluble hydroxy lower alkyl celluloses, such as hydroxy propyl cellulose, are preferred. Additionally (or alternatively) the spheroids may contain a water insoluble polymer, especially an acrylic polymer, an acrylic copolymer, such as a methacrylic acid-ethyl acrylate copolymer, or ethyl cellulose. Other thickening agents or binders include: the lipid type, among which are vegetable oils (cotton seed, sesame and groundnut oils) and derivatives of these oils (hydrogenated oils such as hydrogenated castor oil, glycerol behenate, the waxy type such as natural camauba wax or natural beeswax, synthetic waxes such as cetyl ester waxes, the amphiphilic type such as polymers of ethylene oxide (polyoxyethylene glycol of high molecular weight between 4000 and 100000) or propylene and ethylene oxide copolymers (poloxamers), the cellulosic type (semisynthetic derivatives of cellulose, hydroxypropylmethylcellulose, hydroxypropylcellulose, hydroxymethylcellulose, of high molecular weight and high viscosity, gum) or any other polysaccharide such as alginic acid, the polymeric type such as acrylic acid polymers (such as carbomers), and the mineral type such as colloidal silica and bentonite. See, e.g, Examples 12, 24, and 36 herein.

Suitable diluents for the 3-hydroxy-3-methylglutaryl coenzyme A reductase inhibitor(s)/copper antagonist(s) in the pellets, spheroids or core are, e.g., microcrystalline cellulose, lactose, dicalcium phosphate, calcium carbonate, calcium sulphate, sucrose, dextrates, dextrin, dextrose, dicalcium phosphate dihydrate, kaolin, magnesium carbonate, magnesium oxide, maltodextrin, cellulose, microcrystalline cellulose, sorbitol, starches, pregelatinized starch, talc, tricalcium phosphate and lactose. Suitable lubricants are e.g., magnesium stearate and sodium stearyl fumarate. Suitable binding agents include, e.g., hydroxypropyl methylcellulose, polyvidone, and methylcellulose.

Suitable binders that may be included are: gum arabic, gum tragacanth, guar gum, alginic acid, sodium alginate, sodium carboxymethylcellulose, dextrin, gelatin, hydroxyethylcellulose, hydroxypropylcellulose, liquid glucose, magnesium and aluminum. Suitable disintegrating agents are starch, sodium starch glycolate, crospovidone and croscarmalose sodium. Suitable surface active are Poloxamer 188®, polysorbate 80 and sodium lauryl sulfate. Suitable flow aids are talc colloidal anhydrous silica. Suitable lubricants that may be used are glidants (such as anhydrous silicate, magnesium trisilicate, magnesium silicate, cellulose, starch, talc or tricalcium phosphate) or alternatively antifriction agents (such as calcium stearate, hydrogenated vegetable oils, paraffin, magnesium stearate, polyethylene glycol, sodium benzoate, sodium lauryl sulphate, fumaric acid, stearic acid or zinc stearate and talc). Suitable water-soluble polymers are PEG with molecular weights in the range 1000 to 6000.

Delayed release of the composition or formulation of the invention may be achieved through the use of a tablet, pellet, spheroid or core itself, which besides having a filler and binder, other ancillary substances, in particular lubricants and nonstick agents, and disintegrants. Examples of lubricants and nonstick agents are higher fatty acids and their alkali metal and alkaline-earth-metal salts, such as calcium stearate. Suitable disintegrants are, in particular, chemically inert agents, for example, cross-linked polyvinylpyrrolidone, cross-linked sodium carboxymethylcelluloses, and sodium starch glycolate.

Yet further embodiments of the invention include formulations of one or more 3-hydroxy-3-methylglutaryl coenzyme A reductase inhibitors/copper antagonists, for example, one or more copper chelators, incorporated into transdermal drug delivery systems, such as those described in: Transdermal Drug Delivery Systems, Chapter 10. In: Ansel, H. C., Allen, L. V. and Popovich, N. G. Pharmaceutical Dosage Forms and Drug Delivery Systems, 7th Ed., Lippincott 1999, pp. 263 - 278). Transdermal drug delivery systems facilitate the passage of therapeutic quantities of drug substances through the skin and into the systemic circulation to exert systemic effects, as originally described (see Stoughton, R. D. Percutaneous absorption, Toxicol Appl Pharmacol 7:1-8 (1965)). Evidence of percutaneous drug absorption may be found through measurable blood levels of the drug, detectable excretion of the drug and/or its metabolites in the urine, and through the clinical response of the subject to its administration. For transdermal drug delivery, it is considered ideal if the drug penetrates through the skin to the underlying blood supply without drug build up in the dermal layers (Black, C. D., “Transdermal drug delivery systems,” U.S. Pharm 1:49 (1982)). Formulations of drugs suitable for trans-dermal delivery are known to those skilled in the art, and are described in references such as Ansel et al., (supra). Methods known to enhance the delivery of drugs by the percutaneous route include chemical skin penetration enhancers, which increase skin permeability by reversibly damaging or otherwise altering the physicochemical nature of the stratum corneum to decrease its resistance to drug diffusion (see Shah, V., Peck, C. C., and Williams, R. L., Skin penetration enhancement: clinical pharmacological and regulatory considerations, In: Walters, K. A. and Hadgraft, J. (Eds.) Pharmaceutical skin penetration enhancement. New York: Dekker, 1993). Among effective alterations are increased hydration of the stratum corneum and/or a change in the structure of the lipids and lipoproteins in the intercellular channels brought about through solvent action or denaturation (see Walters K. A., “Percutaneous absorption and transdermal therapy,” Pharm Tech 10:30-42 (1986)). Skin penetration enhancers suitable for formulation with 3-hydroxy-3-methylglutaryl coenzyme A reductase inhibitor/copper antagonist in transdermal drug delivery systems may be chosen from the following list: acetone, laurocapram, dimethylacetamide, dimethylformamide, dimethylsulphoxide, ethanol, oleic acid, polyethylene glycol, propylene glycol and sodium lauryl sulphate. Further skin penetration enhancers may be found in publications known to those skilled in the art (see, for example, Osborne, D. W., & Henke, J. J., “Skin penetration enhancers cited in the technical literature,” Pharm Tech 21:50-66 (1997); Rolf, D., “Chemical and physical methods of enhancing transdermal drug delivery,” Pharm Tech 12:130-139 (1988)).

In addition to chemical means, there are physical methods that enhance transdermal drug delivery and penetration of the compounds and formulations of the invention. These include iontophoresis and sonophoresis. lontophoresis involves the delivery of charged chemical compounds across the skin membrane using an applied electrical field. Such methods have proven suitable for delivery of a number of drugs. Accordingly, another embodiment of the invention comprises one or more 3-hydroxy-3-methylglutaryl coenzyme A reductase inhibitors/copper antagonists, for example, one or more copper chelators, formulated in such a manner suitable for administration by iontophoresis or sonophoresis. Formulations suitable for administration by iontophoresis or sonophoresis may be in the form of gels, creams, or lotions. Transdermal delivery, methods or formulations of the invention, may utilize, among others, monolithic delivery systems, drug-impregnated adhesive delivery systems (e.g., the Latitude™ drug-in-adhesive system from 3M), active transport devices and membrane-controlled systems. Monolithic systems of the invention incorporate a 3-hydroxy-3-methylglutaryl coenzyme A reductase inhibitor/copper antagonist matrix, comprising a polymeric material in which the 3-hydroxy-3-methylglutaryl coenzyme A reductase inhibitor/copper antagonist is dispersed between backing and frontal layers. Drug impregnated adhesive delivery systems comprise an adhesive polymer in which one or more compositions and formulations of the invention and any excipients are incorporated into the adhesive polymer. Active transport devices incorporate a 3-hydroxy-3-methylglutaryl coenzyme A reductase inhibitor/copper antagonist reservoir, often in liquid or gel form, a membrane that may be rate controlling, and a driving force to propel the 3-hydroxy-3-methylglutaryl coenzyme A reductase inhibitor/copper antagonist across the membrane. Membrane-controlled transdermal systems of the invention comprise a 3-hydroxy-3-methylglutaryl coenzyme A reductase inhibitor/copper antagonist reservoir(s), often in liquid or gel form, a membrane that may be rate controlling and backing, adhesive and/or protecting layers. Transdermal delivery dosage forms of the invention include those which substitute the 3-hydroxy-3-methylglutaryl coenzyme A reductase inhibitor/copper antagonist, for the diclofenic or other pharmaceutically acceptable salt thereof referred to in the transdermal delivery systems disclosed in, by way of example, U.S. Pat. Nos. 6,193,996, and 6,262,121.

Formulations and/or compositions for topical administration of one or more compositions and formulations of the invention ingredient can be prepared as an admixture or other pharmaceutical formulation to be applied in a wide variety of ways including, but are not limited to, lotions, creams gels, sticks, sprays, ointments and pastes. These product types may comprise several types of formulations including, but not limited to solutions, emulsions, gels, solids, and liposomes. If the topical composition of the invention is formulated as an aerosol and applied to the skin as a spray-on, a propellant may be added to a solution composition. Suitable propellants as used in the art can be utilized. By way of example of topical administration of an active agent, reference is made to U.S. Pat. Nos. 5,602,125, 6,426,362 and 6,420,411.

Also included in the dosage forms in accordance with the present invention are any variants of the oral dosage forms that are adapted for suppository or other parenteral use. When rectally administered in the form of suppositories, for example, these compositions may be prepared by mixing one or more compounds and formulations of the invention with a suitable non-irritating excipient, such as cocoa butter, synthetic glyceride esters or polyethylene glycols, which are solid at ordinary temperatures, but liquify and/or dissolve in the rectal cavity to release the 3-hydroxy-3-methylglutaryl coenzyme A reductase inhibitor/copper antagonist. Suppositories are generally solid dosage forms intended for insertion into body orifices including rectal, vaginal and occasionally urethrally and can be long acting or slow release. Suppositories include a base that can include, but is not limited to, materials such as alginic acid, which will prolong the release of the pharmaceutically acceptable active ingredient over several hours (5-7). Such bases can be characterized into two main categories and a third miscellaneous group: 1) fatty or oleaginous bases, 2) water-soluble or water-miscible bases and 3) miscellaneous bases, generally combinations of lipophilic and hydrophilic substances. Fatty or oleaginous bases include hydrogenated fatty acids of vegetable oils such as palm kernel oil and cottonseed oil, fat-based compound containing compounds of glycerin with the higher molecular weight fatty acids such as palmitic and stearic acids, cocoa butter is also used where phenol and chloral hydrate lower the melting point of cocoa butter when incorporated, solidifying agents like cetyl esters wax (about 20%) or beeswax (about 4%) may be added to maintain a solid suppository. Other bases include other commercial products such as Fattibase (triglycerides from palm, palm kernel and coconut oils with self-emulsifying glycerol monostearate and poloxyl stearate), Wecobee and Witepsol bases. Water-soluble bases are generally glycerinated gelatin and water-miscible bases are generally polyethylene glycols. The miscellaneous bases include mixtures of the oleaginous and water-soluble or water-miscible materials. An example of such a base in this group is polyoxyl 40 stearate and polyoxyethylene diols and the free glycols.

Transmucosal administration of the compounds and formulations of the invention may utilize any mucosal membrane but commonly utilizes the nasal, buccal, vaginal and rectal tissues.

Formulations suitable for nasal administration of the compounds and formulations of the invention may be administered in a liquid form, for example, nasal spray, nasal drops, or by aerosol administration by nebulizer, including aqueous or oily solutions of the 3-hydroxy-3-methylglutaryl coenzyme A reductase inhibitor/copper antagonist. Formulations for nasal administration, wherein the carrier is a solid, include a coarse powder having a particle size, for example, of less than about 100 microns, preferably less, most preferably one or two times per day than about 50 microns, which is administered in the manner in which snuff is taken, i.e., by rapid inhalation through the nasal passage from a container of the powder held close up to the nose. Compositions in solution may be nebulized by the use of inert gases and such nebulized solutions may be breathed directly from the nebulizing device or the nebulizing device may be attached to a facemask, tent or intermittent positive-pressure breathing machine. Solutions, suspensions or powder compositions of the 3-hydroxy-3-methylglutaryl coenzyme A reductase inhibitor/copper antagonist may be administered orally or nasally from devices that deliver the formulation in an appropriate manner. Formulations of the invention may be prepared as aqueous solutions for example in saline, solutions employing benzyl alcohol or other suitable preservatives, absorption promoters to enhance bio-availability, fluorocarbons, and/or other solubilising or dispersing agents known in the art.

The invention provides extended-release formulations containing one or more 3-hydroxy-3-methylglutaryl coenzyme A reductase inhibitors/copper antagonists, for example, one or more copper chelators, for parenteral administration. Extended rates of 3-hydroxy-3-methylglutaryl coenzyme A reductase inhibitor/copper antagonist action following injection may be achieved in a number of ways, including the following: crystal or amorphous 3-hydroxy-3-methylglutaryl coenzyme A reductase inhibitor/copper antagonist forms having prolonged dissolution characteristics; slowly dissolving chemical complexes of the 3-hydroxy-3-methylglutaryl coenzyme A reductase inhibitor/copper antagonist formulation; solutions or suspensions of 3-hydroxy-3-methylglutaryl coenzyme A reductase inhibitor/copper antagonist in slowly absorbed carriers or vehicles (as oleaginous); increased particle size of 3-hydroxy-3-methylglutaryl coenzyme A reductase inhibitor copper antagonist in suspension; or, by injection of slowly eroding microspheres of 3-hydroxy-3-methylglutaryl coenzyme A reductase inhibitor/copper antagonist (for example, see: Friess, W., Lee, G. and Groves, M. J. Insoluble collagen matrices for prolonged delivery of proteins. Pharmaceut Dev Technol 1:185-193 (1996)). The duration of action of the various forms of insulin for example is based in part on its physical form (amorphous or crystalline), complex formation with added agents, and its dosage form (solution of suspension).

The compositions of the invention can be formulated into a pharmaceutical composition suitable for administration to a patient. See, e.g., Examples 1-36 herein, regarding oral tablets and capsules.

The 3-hydroxy-3-methylglutaryl coenzyme A reductase inhibitor/copper antagonist can be provided and administered in forms suitable for once-a-day dosing. An acetate, phosphate, citrate or glutamate buffer may be added allowing a pH of the final composition to be from about 5.0 to about 9.5; optionally a carbohydrate or polyhydric alcohol tonicifier and, a preservative selected from the group consisting of m-cresol, benzyl alcohol, methyl, ethyl, propyl and butyl parabens and phenol may also be added. Water for injection, tonicifying agents such as sodium chloride, as well as other excipients, may also be present, if desired. For parenteral administration, formulations are isotonic or substantially isotonic to avoid irritation and pain at the site of administration.

The terms buffer, buffer solution and buffered solution, when used with reference to hydrogen-ion concentration or pH, refer to the ability of a system, particularly an aqueous solution, to resist a change of pH on adding acid or alkali, or on dilution with a solvent. Characteristic of buffered solutions, which undergo small changes of pH on addition of acid or base, is the presence either of a weak acid and a salt of the weak acid, or a weak base and a salt of the weak base. An example of the former system is acetic acid and sodium acetate. The change of pH is slight as long as the amount of hydroxyl ion added does not exceed the capacity of the buffer system to neutralize it.

Maintaining the pH of the formulation in the range of approximately 5.0 to 9.5 can enhance the stability of the parenteral formulation of the present invention. Other pH ranges, for example, include, 5.5 to 9.0, or 6.0 to 8.5, or 6.5 to 8.0, or 7.0 to 7.5.

The buffer used in the practice of the present invention is selected from any of the following, for example, an acetate buffer, a phosphate buffer or glutamate buffer, the most preferred buffer being a phosphate buffer.

Carriers or excipients can also be used to facilitate administration of the compositions and formulations of the invention. Examples of carriers and excipients include calcium carbonate, calcium phosphate, various sugars such as lactose, glucose, or sucrose, or types of starch, cellulose derivatives, gelatin, polyethylene glycols and physiologically compatible solvents.

A stabilizer may be included in the formulations of the invention, but will generally not be needed. If included, however, a stabilizer useful in the practice of the invention is a carbohydrate or a polyhydric alcohol. The polyhydric alcohols include such compounds as sorbitol, mannitol, glycerol, xylitol, and polypropylene/ethylene glycol copolymer, as well as various polyethylene glycols (PEG) of molecular weight 200, 400, 1450, 3350, 4000, 6000, and 8000). The carbohydrates include, for example, mannose, ribose, trehalose, maltose, inositol, lactose, galactose, arabinose, or lactose.

The United States Pharmacopeia (USP) states that anti-microbial agents in bacteriostatic or flngistatic concentrations must be added to preparations contained in multiple dose containers. They must be present in adequate concentration at the time of use to prevent the multiplication of microorganisms inadvertently introduced into the preparation while withdrawing a portion of the contents with a hypodermic needle and syringe, or using other invasive means for delivery, such as pen injectors. Antimicrobial agents should be evaluated to ensure compatibility with all other components of the formula, and their activity should be evaluated in the total formula to ensure that a particular agent that is effective in one formulation is not ineffective in another. It is not uncommon to find that a particular agent will be effective in one formulation but not effective in another formulation.

A preservative is, in the common pharmaceutical sense, a substance that prevents or inhibits microbial growth and may be added to a pharmaceutical formulation for this purpose to avoid consequent spoilage of the formulation by microorganisms. While the amount of the preservative is not great, it may nevertheless affect the overall stability of the 3-hydroxy-3-methylglutaryl coenzyme A reductase inhibitor/copper antagonist.

While the preservative for use in the practice of the invention can range from 0.005 to 1.0% (w/v), the preferred range for each preservative, alone or in combination with others, is: benzyl alcohol (0.1-1.0%), or m-cresol (0.1-0.6%), or phenol (0.1-0.8%) or combination of methyl (0.05-0.25%) and ethyl or propyl or butyl (0.005%-0.03%) parabens. The parabens are lower alkyl esters of para-hydroxybenzoic acid.

A detailed description of each preservative is set forth in “Remington's Pharmaceutical Sciences” as well as Pharmaceutical Dosage Forms: Parenteral Medications, Vol. 1, 1992, Avis et al. For these purposes, the 3-hydroxy-3-methylglutaryl coenzyme A reductase inhibitor/copper antagonist may be administered parenterally (including subcutaneous injections, intravenous, intramuscular, intradermal injection or infusion techniques) or by inhalation spray in dosage unit formulations containing conventional non-toxic pharmaceutically acceptable carriers, adjuvants and vehicles.

If desired, the parenteral formulation may be thickened with a thickening agent such as a methylcellulose. The formulation may be prepared in an emulsified form, either water in oil or oil in water. Any of a wide variety of pharmaceutically acceptable emulsifying agents may be employed including, for example, acacia powder, a non-ionic surfactant or an ionic surfactant.

It may also be desirable to add suitable dispersing or suspending agents to the pharmaceutical formulation. These may include, for example, aqueous suspensions such as synthetic and natural gums, e.g., tragacanth, acacia, alginate, dextran, sodium carboxymethylcellulose, methylcellulose, polyvinyl-pyrrolidone or gelatin.

It is possible that other ingredients may be present in the parenteral pharmaceutical formulation of the invention. Such additional ingredients may include wetting agents, oils (e.g., a vegetable oil such as sesame, peanut or olive), analgesic agents, emulsifiers, antioxidants, bulking agents, tonicity modifiers, metal ions, oleaginous vehicles, proteins (e.g., human serum albumin, gelatin or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

Containers and kits are also a part of a composition and may be considered a component. Therefore, the selection of a container is based on a consideration of the composition of the container, as well as of the ingredients, and the treatment to which it will be subjected.

Regarding pharmaceutical formulations, see also, Pharmaceutical Dosage Forms: Parenteral Medications, Vol. 1, 2nd ed., Avis et al., Eds., Mercel Dekker, New York, N.Y. 1992.

Suitable routes of parenteral administration include intramuscular, intravenous, subcutaneous, intraperitoneal, subdermal, intradermal, intraarticular, intrathecal and the like. Mucosal delivery is also permissible. The dose and dosage regimen will depend upon the weight and health of the subject.

In addition to the above means of achieving extended drug action, the rate and duration of 3-hydroxy-3-methylglutaryl coenzyme A reductase inhibitor/copper antagonist delivery may be controlled by, for example by using mechanically controlled drug infusion pumps.

The 3-hydroxy-3-methylglutaryl coenzyme A reductase inhibitor(s)/copper antagonist(s), such as, for example, a copper chelator(s), can be administered in the form of a depot injection that may be formulated in such a manner as to permit a sustained release of the 3-hydroxy-3-methylglutaryl coenzyme A reductase inhibitor/copper antagonist. The 3-hydroxy-3-methylglutaryl coenzyme A reductase inhibitor/copper antagonist can be compressed into pellets or small cylinders and implanted subcutaneously or intramuscularly. The pellets or cylinders may additionally be coated with a suitable biodegradable polymer chosen so as to provide a desired release profile. The 3-hydroxy-3-methylglutaryl coenzyme A reductase inhibitor/copper antagonist may alternatively be micropelleted. The 3-hydroxy-3-methylglutaryl coenzyme A reductase inhibitor/copper antagonist micropellets using bioacceptable polymers can be designed to allow release rates to be manipulated to provide a desired release profile. Alternatively, injectable depot forms can be made by forming microencapsulated matrices of the 3-hydroxy-3-methylglutaryl coenzyme A reductase inhibitor/copper antagonist in biodegradable polymers such as polylactide-polyglycolide. Depending on the ratio of 3-hydroxy-3-methylglutaryl coenzyme A reductase inhibitor/copper antagonist to polymer, and the nature of the particular polymer employed, the rate of 3-hydroxy-3-methylglutaryl coenzyme A reductase inhibitor/copper antagonist release can be controlled. Examples of other biodegradable polymers include poly(orthoesters) and poly(anhydrides). Depot injectable formulations can also be prepared by entrapping the 3-hydroxy-3-methylglutaryl coenzyme A reductase inhibitor/copper antagonist in liposomes, examples of which include unilamellar vesicles, large unilamellar vesicles and multilamellar vesicles. Liposomes can be formed from a variety of phospholipids, such as cholesterol, stearyl amine or phosphatidylcholines. Depot injectable formulations can also be prepared by entrapping the 3-hydroxy-3-methylglutaryl coenzyme A reductase inhibitor/copper antagonist in microemulsions that are compatible with body tissue. By way of example. reference is made to U.S. Pat. Nos. 6,410,041 and 6,362,190.

The invention in part provides infusion dose delivery formulations and devices, including but not limited to implantable infusion devices for delivery of compositions and formulations of the invention. Implantable infusion devices may employ inert material such as biodegradable polymers listed above or synthetic silicones, for example, cylastic, silicone rubber or other polymers manufactured by the Dow-Corning Corporation. The polymer may be loaded with 3-hydroxy-3-methylglutaryl coenzyme A reductase inhibitor/copper antagonist and any excipients. Implantable infusion devices may also comprise a coating of, or a portion of, a medical device wherein the coating comprises the polymer loaded with 3-hydroxy-3-methylglutaryl coenzyme A reductase inhibitor/copper antagonist and any excipient. Such an implantable infusion device may be prepared as disclosed in U.S. Pat. No. 6,309,380 by coating the device with an in vivo biocompatible and biodegradable or bioabsorbable or bioerodible liquid or gel solution containing a polymer with the solution comprising a desired dosage amount of 3-hydroxy-3-methylglutaryl coenzyme A reductase inhibitor/copper antagonist and any excipients. The solution is converted to a film adhering to the medical device thereby forming the implantable 3-hydroxy-3-methylglutaryl coenzyme A reductase inhibitor/copper antagonist-deliverable medical device.

An implantable infusion device may also be prepared by the in situ formation of a 3-hydroxy-3-methylglutaryl coenzyme A reductase inhibitor/copper antagonist containing solid matrix as disclosed in U.S. Pat. No. 6,120,789, herein incorporated in its entirety. Implantable infusion devices may be passive or active. An active implantable infusion device may comprise a 3-hydroxy-3-methylglutaryl coenzyme A reductase inhibitor/copper antagonist reservoir, a means of allowing the 3-hydroxy-3-methylglutaryl coenzyme A reductase inhibitor/copper antagonist to exit the reservoir, for example a permeable membrane, and a driving force to propel the 3-hydroxy-3-methylglutaryl coenzyme A reductase inhibitor/copper antagonist from the reservoir. Such an active implantable infusion device may additionally be activated by an extrinsic signal, such as that disclosed in WO 02/45779, wherein the implantable infusion device comprises a system configured to deliver the 3-hydroxy-3-methylglutaryl coenzyme A reductase inhibitor/copper antagonist comprising an external activation unit operable by a user to request activation of the implantable infusion device, including a controller to reject such a request prior to the expiration of a lockout interval. Examples of an active implantable infusion device include implantable drug pumps. Implantable drug pumps include, for example, miniature, computerized, programmable, refillable drug delivery systems with an attached catheter that inserts into a target organ system, usually the spinal cord or a vessel. See Medtronic Inc. Publications: UC9603124EN NP-2687, 1997; UC199503941b EN NP-2347 182577-101,2000; UC199801017a EN NP3273a 182600-101, 2000; UC200002512 EN NP4050, 2000; UC199900546bEN NP-3678EN, 2000. Minneapolis, Minn: Medtronic Inc; 1997-2000. Many pumps have 2 ports: one into which drugs can be injected and the other that is connected directly to the catheter for bolus administration or analysis of fluid from the catheter. Implantable drug infusion pumps (SynchroMed EL and Synchromed programmable pumps; Medtronic) are indicated for long-term intrathecal infusion of morphine sulfate for the treatment of chronic intractable pain; intravascular infusion of floxuridine for treatment of primary or metastatic cancer; intrathecal injection (baclofen injection) for severe spasticity; long-term epidural infusion of morphine sulfate for treatment of chronic intractable pain; long-term intravascular infusion of doxorubicin, cisplatin, or methotrexate for the treatment or metastatic cancer; and long-term intravenous infusion of clindamycin for the treatment of osteomyelitis. Such pumps may also be used for the long-term infusion of one or more 3-hydroxy-3-methylglutaryl coenzyme A reductase inhibitor/copper antagonists, for example, one or more copper chelators, at a desired amount for a desired number of doses or steady state administration. One form of a typical implantable drug infusion pump (Synchromed EL programmable pump; Medtronic) is titanium covered and roughly disk shaped, measures 85.2 mm in diameter and 22.86 mm in thickness, weighs 185 g, has a drug reservoir of 10 mL, and runs on a lithium thionyl-chloride battery with a 6- to 7-year life, depending on use. The downloadable memory contains programmed drug delivery parameters and calculated amount of drug remaining, which can be compared with actual amount of drug remaining to access accuracy of pump function, but actual pump function over time is not recorded. The pump is usually implanted in the right or left abdominal wall. Other pumps useful in the invention include, for example, portable disposable infuser pumps (PDIPs). Additionally, implantable infusion devices may employ liposome delivery systems, such as a small unilamellar vesicles, large unilamellar vesicles, and multilamellar vesicles can be formed from a variety of phospholipids, such as cholesterol, stearyl amine or phosphatidylcholines.

The invention also includes delayed-release ocular preparations containing one or more 3-hydroxy-3-methylglutaryl coenzyme A reductase inhibitors/copper antagonists, for example, one or more copper chelators. One of the problems associated with the use of ophthalmic solutions is the rapid loss of administered drug due to blinking of the eye and the flushing effect of lacrimal fluids. Up to 80% of an administered dose may be lost through tears and the action of nasolacrimal drainage within 5 minutes of installation. Extended periods of therapy may be achieved by formulations of the invention that increase the contact time between the 3-hydroxy-3-methylglutaryl coenzyme A reductase inhibitor/copper antagonist and the corneal surface. This may be accomplished through use of agents that increase the viscosity of solutions; by ophthalmic suspensions in which the 3-hydroxy-3-methylglutaryl coenzyme A reductase inhibitor/copper antagonist particles slowly dissolve; by slowly dissipating ophthalmic ointments; or by use of ophthalmic inserts. Preparations of one or more 3-hydroxy-3-methylglutaryl coenzyme A reductase inhibitors/copper antagonists, for example, one or more copper chelators, suitable for ocular administration to humans may be formulated using synthetic high molecular weight cross-linked polymers such as those of acrylic acid (e.g., Carbopol 940) or gellan gum (Gelrite; see, Merck Index 12th Ed., 4389), a compound that forms a gel upon contact with the precorneal tear film (e.g. as employed in Timoptic-XE by Merck, Inc.).

Further examples include delayed-release ocular preparations containing 3-hydroxy-3-methylglutaryl coenzyme A reductase inhibitor/copper antagonist in ophthalmic inserts, such as the OCUSERT system (Alza Inc.). Typically, such inserts are elliptical with dimensions of about 13.4 mm by 5.4 mm by 0.3 mm (thickness). The insert is flexible and has a 3-hydroxy-3-methylglutaryl coenzyme A reductase inhibitor/copper antagonist -containing core surrounded on each side by a layer of hydrophobic ethylene/vinyl acetate copolymer membranes through which the 3-hydroxy-3-methylglutaryl coenzyme A reductase inhibitor/copper antagonist diffuses at a constant rate. The white margin around such devices contains white titanium dioxide, an inert compound that confers visibility. The rate of 3-hydroxy-3-methylglutaryl coenzyme A reductase inhibitor/copper antagonist diffusion is controlled by the polymer composition, the membrane thickness, and the copper antagonist solubility. During the first few hours after insertion, the 3-hydroxy-3-methylglutaryl coenzyme A reductase inhibitor/copper antagonist release rate is greater than that which occurs thereafter in order to achieve initially therapeutic 3-hydroxy-3-methylglutaryl coenzyme A reductase inhibitor/copper antagonist levels. The 3-hydroxy-3-methylglutaryl coenzyme A reductase inhibitor/copper antagonist -containing inserts may be placed in the conjunctival sac from which they release their medication over a treatment period. Another form of an ophthalmic insert is a rod shaped, water-soluble structure composed of hydroxypropyl cellulose in which 3-hydroxy-3-methylglutaryl coenzyme A reductase inhibitor/copper antagonist is embedded. The insert is placed into the inferior cul-de-sac of the eye once or twice daily as required for therapeutic efficacy. The inserts soften and slowly dissolve, releasing the 3-hydroxy-3-methylglutaryl coenzyme A reductase inhibitor/copper antagonist that is then taken up by the ocular fluids. A further example of such a device is that furnished by Lacrisert (Merck Inc.).

The invention also provides in part dose delivery formulations and devices formulated to enhance bioavailability of 3-hydroxy-3-methylglutaryl coenzyme A reductase inhibitor/copper antagonist. This may be in addition to or in combination with any of the formulations or devices described above.

Despite good hydrosolubility, one or more 3-hydroxy-3-methylglutaryl coenzyme A reductase inhibitors/copper antagonists, such as a copper chelator, for example, trientine, may be poorly absorbed in the digestive tract. A therapeutically effective amount of 3-hydroxy-3-methylglutaryl coenzyme A reductase inhibitor/copper antagonist is an amount capable of providing an appropriate level of 3-hydroxy-3-methylglutaryl coenzyme A reductase inhibitor/copper antagonist in the bloodstream. By increasing the bioavailability of 3-hydroxy-3-methylglutaryl coenzyme A reductase inhibitor i copper antagonist, a therapeutically effective level of 3-hydroxy-3-methylglutaryl coenzyme A reductase inhibitor/copper antagonist may be achieved by administering lower dosages than would otherwise be necessary.

An increase in bioavailability of 3-hydroxy-3-methylglutaryl coenzyme A reductase inhibitor/copper antagonist may be achieved by complexation of 3-hydroxy-3-methylglutaryl coenzyme A reductase inhibitor/copper antagonist with one or more bioavailability or absorption enhancing agents or in bioavailability or absorption enhancing formulations.

The invention in part provides for the formulation of 3-hydroxy-3-methylglutaryl coenzyme A reductase inhibitor/copper antagonist, e.g., copper chelator, with other agents useful to enhance bioavailability or absorption. Such bioavailability or absorption enhancing agents include, but are not limited to, various surfactants such as various triglycerides, such as from butter oil, monoglycerides, such as of stearic acid and vegetable oils, esters thereof, esters of fatty acids, propylene glycol esters, the polysorbates, sodium lauryl sulfate, sorbitan esters, sodium sulfosuccinate, among other compounds. By altering the surfactant properties of the delivery vehicle it is possible to, for example, allow a 3-hydroxy-3-methylglutaryl coenzyme A reductase inhibitor/copper antagonist to have greater intestinal contact over a longer period of time that increases uptake and reduces side effects. Further examples of such agents include carrier molecules such as cyclodextrin and derivatives thereof, well known in the art for their potential as complexation agents capable of altering the physicochemical attributes of drug molecules. For example, cyclodextrins may stabilize (both thermally and oxidatively), reduce the volatility of, and alter the solubility of, 3-hydroxy-3-methylglutaryl coenzyme A reductase inhibitor/copper antagonist with which they are complexed. Cyclodextrins are cyclic molecules composed of glucopyranose ring units that form toroidal structures. The interior of the cyclodextrin molecule is hydrophobic and the exterior is hydrophilic, making the cyclodextrin molecule water-soluble. The degree of solubility can be altered through substitution of the hydroxyl groups on the exterior of the cyclodextrin. Similarly, the hydrophobicity of the interior can be altered through substitution, though generally the hydrophobic nature of the interior allows accommodation of relatively hydrophobic guests within the cavity. Accommodation of one molecule within another is known as complexation and the resulting product is referred to as an inclusion complex. Examples of cyclodextrin derivatives include sulfobutylcyclodextrin, maltosylcyclodextrin, hydroxypropylcyclodextrin, and salts thereof. Complexation of 3-hydroxy-3-methylglutaryl coenzyme A reductase inhibitor/copper antagonist with a carrier molecule such as cyclodextrin to form an inclusion complex may thereby reduce the size of the 3-hydroxy-3-methylglutaryl coenzyme A reductase inhibitor/copper antagonist dose needed for therapeutic efficacy by enhancing the bioavailability of the administered active agent.

The invention in part also provides for the formulation of 3-hydroxy-3-methylglutaryl coenzyme A reductase inhibitor/copper antagonist, e.g., copper chelator, in a microemulsion to enhance bioavailability. A microemulsion is a fluid and stable homogeneous solution composed of four major constituents, respectively, a hydrophilic phase, a lipophilic phase, at least one surfactant (SA) and at least one cosurfactant (CoSA). A surfactant is a chemical compound possessing two groups, the first polar or ionic, which has a great affinity for water, the second which contains a longer or shorter aliphatic chain and is hydrophobic. These chemical compounds having marked hydrophilic character are intended to cause the formation of micelles in aqueous or oily solution. Examples of suitable surfactants include mono-, di- and triglycerides and polyethylene glycol (PEG) mono- and diesters. A cosurfactant, also sometimes known as “co-surface-active agent”, is a chemical compound having hydrophobic character, intended to cause the mutual solubilization of the aqueous and oily phases in a microemulsion. Examples of suitable co-surfactants include ethyl diglycol, lauric esters of propylene glycol, oleic esters of polyglycerol, and related compounds.

The invention in part also provides for the formulation of 3-hydroxy-3-methylglutaryl coenzyme A reductase inhibitors/copper antagonists with various polymers to enhance bioavailability by increasing adhesion to mucosal surfaces, by decreasing the rate of degradation by hydrolysis or enzymatic degradation of the 3-hydroxy-3-methylglutaryl coenzyme A reductase inhibitor/copper antagonist, and by increasing the surface area of the 3-hydroxy-3-methylglutaryl coenzyme A reductase inhibitor/copper antagonist relative to the size of the particle. Suitable polymers can be natural or synthetic, and can be biodegradable or non-biodegradable. Delivery of low molecular weight active agents, such as for example 3-hydroxy-3-methylglutaryl coenzyme A reductase inhibitor/copper antagonist, including compounds of Formulae I, I(a) and II and trientine active agents, may occur by either diffusion or degredation of the polymeric system. Representative natural polymers include proteins such as zein, modified zein, casein, gelatin, gluten, serum albumin, and collagen, polysaccharides such as cellulose, dextrans, and polyhyaluronic acid. Synthetic polymers are generally preferred due to the better characterization of degradation and release profiles. Representative synthetic polymers include polyphosphazenes, poly(vinyl alcohols), polyamides, polycarbonates, polyacrylates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes and copolymers thereof. Examples of suitable polyacrylates include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate) and poly(octadecyl acrylate). Synthetically modified natural polymers include cellulose derivatives such as alkyl celluloses, hydroxyalkyl celluloses, cellulose ethers, cellulose esters, and nitrocelluloses. Examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate and cellulose sulfate sodium salt. Each of the polymers described above can be obtained from commercial sources such as Sigma Chemical Co., St. Louis, Mo., Polysciences, Warrenton, Pa., Aldrich Chemical Co., Milwaukee, Wis., Fluka, Ronkonkoma, N.Y., and BioRad, Richmond, Calif. or can be synthesized from monomers obtained from these suppliers using standard techniques. The polymers described above can be separately characterized as biodegradable, non-biodegradable, and bioadhesive polymers, as discussed in more detail below. Representative synthetic degradable polymers include polyhydroxy acids such as polylactides, polyglycolides and copolymers thereof, poly(ethylene terephthalate), poly(butic acid), poly(valeric acid), poly(lactide-co-caprolactone), polyanhydrides, polyorthoesters and blends and copolymers thereof. Representative natural biodegradable polymers include polysaccharides such as alginate, dextran, cellulose, collagen, and chemical derivatives thereof (substitutions, additions of chemical groups, for example, alkyl, alkylene, hydroxylations, oxidations, and other modifications routinely made by those skilled in the art), and proteins such as albumin, zein and copolymers and blends thereof, alone or in combination with synthetic polymers. In general, these materials degrade either by enzymatic hydrolysis or exposure to water in vivo, by surface or bulk erosion. Examples of non-biodegradable polymers include ethylene vinyl acetate, poly(meth)acrylic acid, polyamides, polyethylene, polypropylene, polystyrene, polyvinyl chloride, polyvinylphenol, and copolymers and mixtures thereof. Hydrophilic polymers and hydrogels tend to have bioadhesive properties. Hydrophilic polymers that contain carboxylic groups (e.g., poly[acrylic acid]) tend to exhibit the best bioadhesive properties. Polymers with the highest concentrations of carboxylic groups are preferred when bioadhesiveness on soft tissues is desired. Various cellulose derivatives, such as sodium alginate, carboxymethylcellulose, hydroxymethylcellulose and methylcellulose also have bioadhesive properties. Some of these bioadhesive materials are water-soluble, while others are hydrogels. Polymers such as hydroxypropylmethylcellulose acetate succinate (HPMCAS), cellulose acetate trimellitate (CAT), cellulose acetate phthalate (CAP), hydroxypropylcellulose acetate phthalate (HPCAP), hydroxypropylmethylcellulose acetate phthalate (HPMCAP), and methylcellulose acetate phthalate (MCAP) may be utilized to enhance the bioavailability of 3-hydroxy-3-methylglutaryl coenzyme A reductase inhibitor/copper antagonist with which they are complexed. Rapidly bioerodible polymers such as poly(lactide-co-glycolide), polyanhydrides, and polyorthoesters, whose carboxylic groups are exposed on the external surface as their smooth surface erodes, can also be used for bioadhesive 3-hydroxy-3-methylglutaryl coenzyme A reductase inhibitor/copper antagonist delivery systems. In addition, polymers containing labile bonds, such as polyanhydrides and polyesters, are well known for their hydrolytic reactivity. Their hydrolytic degradation rates can generally be altered by simple changes in the polymer backbone. Upon degradation, these materials also expose carboxylic groups on their external surface, and accordingly, these can also be used for bioadhesive 3-hydroxy-3-methylglutaryl coenzyme A reductase inhibitor/copper antagonist delivery systems.

Other agents that may enhance bioavailability or absorption of one or more 3-hydroxy-3-methylglutaryl coenzyme A reductase inhibitors/copper antagonists can act by facilitating or inhibiting transport across the intestinal mucosa. For example, it has long been suggested that blood flow in the stomach and intestine is a factor in determining intestinal drug absorption and drug bioavailability, so that agents that increase blood flow, such as vasodilators, may increase the rate of absorption of orally administered 3-hydroxy-3-methylglutaryl coenzyme A reductase inhibitor/copper antagonist by increasing the blood flow to the gastrointestinal tract. Vasodilators have been used in combination with other drugs. For example, in EPO Publication 106335, the use of a coronary vasodilator, diltiazem, is reported to increase oral bioavailability of drugs which have an absolute bioavailability of not more than 20%, such as adrenergic beta-blocking agents (e.g., propranolol), catecholamines (e.g., dopamine), benzodiazepine derivatives (e.g., diazepam), vasodilators (e.g., isosorbide dinitrate, nitroglycerin or amyl nitrite), cardiotonics or antidiabetic agents, bronchodilators (e.g., tetrahydroisoquinoline), hemostatics (e.g., carbazochrome sulfonic acid), antispasmodics (e.g., timepidium halide) and antitussives (e.g., tipepidine). Vasodilators therefore constitute another class of agents that may enhance the bioavailability of 3-hydroxy-3-methylglutaryl coenzyme A reductase inhibitor/copper antagonist.

Other mechanisms of enhancing bioavailability of the compositions and formulations of the invention include the inhibition of reverse active transport mechanisms. For example, it is now thought that one of the active transport mechanisms present in the intestinal epithelial cells is p-glycoprotein transport mechanism which facilitates the reverse transport of substances, which have diffused or have been transported inside the epithelial cell, back into the lumen of the intestine. It has been speculated that the p-glycoprotein present in the intestinal epithelial cells may function as a protective reverse pump which prevents toxic substances which have been ingested and diffused or transported into the epithelial cell from being absorbed into the circulatory system and becoming bioavailable. One of the unfortunate aspects of the function of the p-glycoprotein in the intestinal cell however is that it can also function to prevent bioavailability of substances which are beneficial, such as certain drugs which happen to be substrates for the p-glycoprotein reverse transport system. Inhibition of this p-glycoprotein mediated active transport system will cause less drug to be transported back into the lumen and will thus increase the net drug transport across the gut epithelium and will increase the amount of drug ultimately available in the blood. Various p-glycoprotein inhibitors are well known and appreciated in the art. These include, water soluble vitamin E; polyethylene glycol; poloxamers including Pluronic F-68; Polyethylene oxide; polyoxyethylene castor oil derivatives including Cremophor EL and Cremophor RH 40; Chrysin, (+)-Taxifolin; Naringenin; Diosmin; Quercetin; and the like. Inhibition of a reverse active transport system of which, for example, a 3-hydroxy-3-methylglutaryl coenzyme A reductase inhibitor/copper antagonist is a substrate may thereby enhance the bioavailability of said 3-hydroxy-3-methylglutaryl coenzyme A reductase inhibitor/copper antagonist.

A better understanding of the invention will be gained by reference to the following experimental section. The following experiments are illustrative and are not intended to limit the invention or the claims in any way.

### EXAMPLE 1

### Combination Tablet

This Example describes preparation of tablets having a copper antagonist(s) such as, for example, one or more copper chelators (e.g., a trientine, such as triethylenetetramine dihydrochloride or triethylenetetramine disuccinate), and one or more 3-hydroxy-3-methylglutaryl coenzyme A reductase inhibitors, for example, statins (e.g., simvastatin), which may be prepared using. compaction and direct compression methods.

Ingredients for tablets including, for example, triethylenetetramine dihydrochloride or triethylenetetramine disuccinate and simvastatin, are provided in the below table:

| | Exemplary | Mg per | | Kg per | |
|---|---|---|---|---|---|
| Ingredient | Function | tablet | % | 10 kg | |
| Triethylenetetramine | Copper | 300 | 95.81 | 9.581 | |
| dihydrochloride or | binding | | | | |
| triethylenetetramine | | | | | |
| disuccinate | | | | | |
| Simvastatin | HMG-CoA | 10 | 3.19 | 0.319 | |
| | reductase | | | | |
| | inhibitor | | | | |
| Silicon dioxide | Glidant; | 1.57 | 0.50 | 0.05 | |
| | desiccant | | | | |
| Magnesium stearate | Lubricant | 1.57 | 0.50 | 0.05 | |
| Total | | 313.14 | 100 | 10 | Kg |

The process employs compaction and direct compression. The copper antagonist(s) is/are first compacted, for example, in a suitable roller compacter. Suitable roller compactors include, for example, the Vector Mini-Model TF (Vector Corp., Marion, Iowa). It is then milled with a Fitz mill (Fitzpatrick Company, Elmhurst, Ill.) or other suitable mill, such as a Quadro Comill, oscillator mill, or pin mill, for example.

The milled copper antagonist is blended with the statin, silicon dioxide and magnesium stearate, for example, in a suitable blender. Suitable blenders include v-Blenders (Patterson-Kelly), planetary blenders (Hobart Corp., Troy Ohio).

The final blend is compressed into tablets using a suitable tablet machine, such as a Manesty beta-press (Manesty, Knowsley, Merseyside, UK).

Copper antagonists other than triethylenetetramine dihydrochloride or triethylenetetramine disuccinate may be used, as may statins other than simvastatin. Additionally, in certain cases, a copper antagonist compound which has already been considerably precomplexed with a non-copper metal ion as disclosed herein may also be used, for example, a triethylenetetramine precomplexed with calcium or another non-copper metal ion. Pentacoordinate copper antagonists may also be used, including for example, a triethylenetetramine complexed with calcium (or another non-copper metal) and another complexing agent, such as, for example, chloride, as disclosed herein.

Amounts of the copper antagonist(s) and statin(s), including the amounts of triethylenetetramine dihydrochloride or triethylenetetramine disuccinate and simvastatin set forth in this Example, may be varied, as appropriate. By way of example only, the amount of triethylenetetramine dihydrochloride or triethylenetetramine disuccinate (or other copper antagonist) may range from about 1 mg to about 750 mg (for example, 1 mg, 5 mg, 10 mg, 25 mg, 50 mg, 100 mg, 125 mg, 150 mg 200 mg, 250 mg, 400 mg, 500 mg, 600 mg, and 750 mg), and the amount of simvastatin may range from about 1 mg to about 40 mg (for example, 1 mg, 2 mg, 2.5 mg, 3 mg, 4 mg, 5 mg, 10 mg, 20 mg, 25 mg, 30 mg, and 40 mg). Other amounts may also be used. The amounts are not inflexible and may be determined, in part, for example, based on the number of tablets to be taken per day.

The tablet may be prepared for administration of drug, by way of example, in one or more doses, for example, one or two or more tablets once, twice, or more per day. Tablets are generally prepared for administration no more than four times per day, preferably less, and most preferably one or two times per day.

### EXAMPLE 2

### Combination Tablet with Fillers

This Example describes preparation of tablets including fillers having a copper antagonist(s) such as, for example, one or more copper chelators (e.g., a trientine, such as triethylenetetramine dihydrochloride or triethylenetetramine disuccinate), and one or more 3-hydroxy-3-methylglutaryl coenzyme A reductase inhibitors, for example, statins (e.g., simvastatin), which may be prepared using blending and direct compression methods.

Ingredients for tablets including, for example, triethylenetetramine dihydrochloride or triethylenetetramine disuccinate and simvastatin, are provided in the below table:

| | | (i) Mg | | |
|---|---|---|---|---|
| | Exemplary | per | | |
| Ingredient | Function | tablet | % | Kg per 10 Kg |
| Triethylenetetramine | Copper | 300 | 74.52 | 7.452 |
| dihydrochloride or | binding | | | |
| triethylenetetramine | | | | |
| disuccinate | | | | |
| Simvastatin | HMG-CoA | 10 | 2.48 | 0.248 |
| | reductase | | | |
| | inhibitor | | | |
| Microcrystalline | Filler-binder | 80.52 | 20.00 | 2.00 |
| cellulose | | | | |
| Croscarmellose | Disintegrant | 8.05 | 2.00 | 0.20 |
| sodium | | | | |
| Silicon dioxide | Glidant; | 2.01 | 0.50 | 0.05 |
| | desiccant | | | |
| Magnesium stearate | Lubricant | 2.01 | 0.50 | 0.05 |
| Total | | 402.59 | 100 | 10 |

The process employs a combination of blending and direct compression techniques. The copper antagonist is blended, for example, with the statin in a suitable blender. Suitable blenders include, for example, V-Blenders (Patterson-Kelly), planetary blenders (Hobart Corp).

The resulting blend is mixed with microcrystalline cellulose, which may also be done in a suitable blender. This blend is milled and screened in a Fitz mill (Fitzpatrick Corp) or other suitable mill, such as a Quadro Comill, oscillating mill, or pin mill, for example.

The resulting blend is mixed with the silicon dioxide and magnesium stearate, which may also be accomplished in a suitable blender.

The final blend is compressed into tablets on a suitable tablet machine, such as a Manesty beta-press.

Copper antagonists other than triethylenetetramine dihydrochloride or triethylenetetramine disuccinate may be used, as may statins other than simvastatin. Additionally, in certain cases, a copper antagonist compound which has already been considerably precomplexed with a non-copper metal ion as disclosed herein may also be used, for example, a triethylenetetramine precomplexed with calcium or another non-copper metal ionmetal ion as disclosed herein may be used, for example a triethylenetetramine precomplexed with calcium or another non-copper metal ion. Pentacoordinate copper antagonists may also be used, including for example, a triethylenetetramine complexed with calcium (or another non-copper metal) and another complexing agent, such as, for example, chloride, as disclosed herein.

Amounts of the copper antagonist(s) and statin(s), including the amounts of triethylenetetramine dihydrochloride or triethylenetetramine disuccinate and simvastatin set forth in this Example, may be varied, as appropriate. By way of example only, the amount of triethylenetetramine dihydrochloride or triethylenetetramine disuccinate (or other copper antagonist) may range from about 1 mg to about 750 mg (for example, 1 mg, 5 mg, 10 mg, 25 mg, 50 mg, 100 mg, 125 mg, 150 mg 200 mg, 250 mg, 400 mg, 500 mg, 600 mg, and 750 mg), and the amount of simvastatin may range from about 1 mg to about 40 mg (for example, 1 mg, 2 mg, 2.5 mg, 3 mg, 4 mg, 5 mg, 10 mg, 20 mg, 25 mg, 30 mg, and 40 mg). Other amounts may also be used. The amounts are not inflexible and may be determined, in part, for example, based on the number of tablets to be taken per day.

The tablet may be prepared for administration of drug, by way of example, in one or more doses, for example, one or two or more tablets once, twice, or more per day. Tablets are generally prepared for administration no more than four times per day, preferably less, and most preferably one or two times per day.

### EXAMPLE 3

### Combination Tablet with Desiccant(s)

This Example describes preparation of tablets having a copper antagonist(s) such as, for example, one or more copper chelators (e.g., a trientine, such as triethylenetetramine dihydrochloride or triethylenetetramine disuccinate) and one or more 3-hydroxy-3-methylglutaryl coenzyme A reductase inhibitors, for example, statins (e.g., simvastatin), together with one or more desiccants, which may be prepared using direct compression methods.

Ingredients for tablets including, for example, triethylenetetramine dihydrochloride or triethylenetetramine disuccinate and simvastatin, are provided in the below table:

| | Exemplary | Mg per | | Kg per |
|---|---|---|---|---|
| Ingredient | Function | tablet | % | 10 Kg |
| Triethylenetetramine | Copper | 300 | 90.97 | 9.097 |
| dihydrochloride or | binding | | | |
| triethylenetetramine | | | | |
| disuccinate | | | | |
| Simvastatin | HMG-CoA | 10 | 3.03 | 0.303 |
| | reductase | | | |
| | inhibitor | | | |
| Disodium phosphate | Desiccant | 16.49 | 5.0 | 0.50 |
| anhydrous | | | | |
| Silicon dioxide | Glidant; | 1.65 | 0.5 | 0.05 |
| | desiccant | | | |
| Magnesium stearate | Lubricant | 1.65 | 0.5 | 0.05 |
| Total | | 329 | 100 | 10 |

The process employs compaction, blending and direct compression. The copper antagonist(s), statin(s) and desiccant(s) are blended in a suitable blender. Suitable blenders include, for example, v-blenders (Patterson-Kelly), planetary blenders (Hobart). This blend is compacted in a suitable roller compacter, such as a Vector Mini-model TF. It is then milled and screened in a Fitz mill or other suitable mill. Suitable mills include a Quadro Comill, oscillating mills, and pin mills, for example.

The resulting blend is blended with the simvastatin, silicon dioxide and magnesium stearate in a suitable blender.

The final blend is compacted into tablets on a suitable tablet machine, such as a Manesty beta press.

Copper antagonists other than triethylenetetramine dihydrochloride or triethylenetetramine disuccinate may be used, as may statins other than simvastatin. Additionally, in certain cases, a copper antagonist compound which has already been considerably precomplexed with a non-copper metal ion as disclosed herein may also be used, for example, a triethylenetetramine precomplexed with calcium or another non-copper metal ionmetal ion as disclosed herein may be used, for example a triethylenetetramine precomplexed with calcium or another non-copper metal ion. Pentacoordinate copper antagonists may also be used, including for example, a triethylenetetramine complexed with calcium (or another non-copper metal) and another complexing agent, such as, for example, chloride, as disclosed herein.

Amounts of the copper antagonist(s) and statin(s), including the amounts of triethylenetetramine dihydrochloride or triethylenetetramine disuccinate and simvastatin set forth in this Example, may be varied, as appropriate. By way of example only, the amount of triethylenetetramine dihydrochloride or triethylenetetramine disuccinate (or other copper antagonist) may range from about 1 mg to about 750 mg (for example, 1 mg, 5 mg, 10 mg, 25 mg, 50 mg, 100 mg, 125 mg, 150 mg 200 mg, 250 mg, 400 mg, 500 mg, 600 mg, and 750 mg), and the amount of simvastatin may range from about 1 mg to about 40 mg (for example, 1 mg, 2 mg, 2.5 mg, 3 mg, 4 mg, 5 mg, 10 mg, 20 mg, 25 mg, 30 mg, and 40 mg). Other amounts may also be used. The amounts are not inflexible and may be determined, in part, for example, based on the number of tablets to be taken per day.

The tablet may be prepared for administration of drug, by way of example, in one or more doses, for example, one or two or more tablets once, twice, or more per day. Tablets are generally prepared for administration no more than four times per day, preferably less, and most preferably one or two times per day.

### EXAMPLE 4

### Combination Tablet with Wet Granulation Binder(s)

This Example describes preparation of tablets having a copper antagonist(s) such as, for example, one or more copper chelators (e.g., a trientine, such as triethylenetetramine dihydrochloride or triethylenetetramine disuccinate) and one or more 3-hydroxy-3-methylglutaryl coenzyme A reductase inhibitors, for example, statins (e.g., simvastatin), which may be prepared using wet granulation methods.

Ingredients for tablets including, for example, triethylenetetramine dihydrochloride or triethylenetetramine disuccinate and simvastatin, are provided in the below table:

| | Exemplary | Mg per | | Kg per |
|---|---|---|---|---|
| Ingredient | Function | Tablet | % | 10 Kg |
| Triethylenetetramine | Copper | 300 | 72.10 | 7.21 |
| dihydrochloride or | binding | | | |
| triethylenetetramine | | | | |
| disuccinate | | | | |
| Simvastatin | HMG-CoA | 10 | 2.40 | 0.240 |
| | reductase | | | |
| | inhibitor | | | |
| Lactose USP | Filler | 41.61 | 10.00 | 1.00 |
| Dicalcium phosphate | Filler | 41.61 | 10.00 | 1.00 |
| Crosscarmellose | Disintegrant | 8.32 | 2.00 | 0.20 |
| sodium | | | | |
| Hydroxypropylcellulose | Granulation | 12.48 | 3.00 | 0.30 |
| | binder | | | |
| Magnesium stearate | Lubricant | 2.06 | 0.50 | 0.05 |
| Total | | 416.08 | 100 | 10.0 |

This tablet is prepared using wet granulation methods. The copper antagonist(s) is blended with the statin(s), lactose and dicalcium phosphate in a suitable fluid bed granulator/dryer. Suitable granulator/dryers include Glatt or Niro fluid bed granulator/dryers. The hydroxypropylcellulose is dissolved in water or ethanol, and the blend is wet granulated with the solution of hydroxypropylcellulose in the suitable granulator/dryer. The wet granulation is dried in the granulator dryer. Where triethylenetetramine dihydrochloride is included in this formulation it is preferably precomplexed with a non-copper metal ion as disclosed herein, e.g., calcium, to enhance stability.

Alternatively this granulation can be prepared by blending the copper antagonist(s) with lactose and dicalcium phosphate and wet granulating with the hydroxypropylcellulose solution in a Niro or Glatt high speed granulator and drying it in a Glatt fluid bed dryer.

This final granulation is mixed with the crosscarmellose sodium and magnesium stearate or other lubricant in a suitable blender, such as Patterson Kelly V-blender.

The final blend is compressed into tablets on a suitable tablet machine, such as a Manesty beta-press.

Copper antagonists other than triethylenetetramine dihydrochloride or triethylenetetramine disuccinate may be used, as may statins other than simvastatin. Additionally, in certain cases, a copper antagonist compound which has already been considerably precomplexed with a non-copper metal ion as disclosed herein may be used, for example a triethylenetetramine precomplexed with calcium or another non-copper metal ion. Pentacoordinate copper antagonists may also be used, including for example, a triethylenetetramine complexed with calcium (or another non-copper metal) and another complexing agent, such as, for example, chloride, as disclosed herein.

Amounts of the copper antagonist(s) and statin(s), including the amounts of triethylenetetramine dihydrochloride or triethylenetetramine disuccinate and simvastatin set forth in this Example, may be varied, as appropriate. By way of example only, the amount of triethylenetetramine dihydrochloride or triethylenetetramine disuccinate (or other copper antagonist) may range from about 1 mg to about 750 mg (for example, 1 mg, 5 mg, 10 mg, 25 mg, 50 mg, 100 mg, 125 mg, 150 mg 200 mg, 250 mg, 400 mg, 500 mg, 600 mg, and 750 mg), and the amount of simvastatin may range from about 1 mg to about 40 mg (for example, 1 mg, 2 mg, 2.5 mg, 3 mg, 4 mg, 5 mg, 10 mg, 20 mg, 25 mg, 30 mg, and 40 mg). Other amounts may also be used. The amounts are not inflexible and may be determined, in part, for example, based on the number of tablets to be taken per day.

The tablet may be prepared for administration of drug, by way of example, in one or more doses, for example, one or two or more tablets once, twice, or more per day. Tablets are generally prepared for administration no more than four times per day, preferably less, and most preferably one or two times per day.

### EXAMPLE 5

### A Combination Tablet Employing a Wet Granulation and a Desiccant

This Example describes preparation of tablets having a copper antagonist(s) such as, for example, one or more copper chelators (e.g., a trientine, such as triethylenetetramine dihydrochloride or triethylenetetramine disuccinate) and one or more 3-hydroxy-3-methylglutaryl coenzyme A reductase inhibitors, for example, statins (e.g., simvastatin), together with one or more desiccants, which may be prepared using wet granulation methods.

Ingredients for tablets including, for example, triethylenetetramine dihydrochloride or triethylenetetramine disuccinate and simvastatin, are provided in the below table:

| | Exemplary | Mg per | | Kg per |
|---|---|---|---|---|
| Ingredient | Function | tablet | % | 10 Kg |
| Triethylenetetramine | Copper | 300 | 67.26 | 6.726 |
| dihydrochloride or | binding | | | |
| triethylenetetramine | | | | |
| disuccinate | | | | |
| Simvastatin | HMG-CoA | 10 | 2.24 | 0.224 |
| | reductase | | | |
| | inhibitor | | | |
| Lactose USP | Filler | 89.21 | 20.00 | 2.00 |
| Crosscarmellose | Disintegrant | 8.92 | 2.00 | 0.200 |
| sodium | | | | |
| Hydroxypropylcellulose | Granulation | 13.38 | 3.00 | 0.300 |
| | Binder | | | |
| Disodium phosphate | Desiccant | 22.30 | 5.00 | 0.500 |
| anhydrous | | | | |
| Magnesium stearate | Lubricant | 2.23 | 0.50 | 0.050 |
| Total | | 446.04 | 100 | 10 |

This tablet is prepared using wet granulation methods. The copper antagonist(s) is blended with the statin(s), and lactose in a suitable fluid bed granulator/dryer. Suitable granulator/dryers include Glatt or Niro fluid bed granulator/dryers. The hydroxypropylcellulose is dissolved in water or ethanol, and the blend is wet granulated with the solution of hydroxypropylcellulose in a suitable granulator/dryer. The wet granulation is dried in the granulator dryer. Where triethylenetetramine dihydrochloride is included in this formulation it is preferably precomplexed with a non-copper metal ion as disclosed herein, e.g., calcium, to enhance stability.

Alternatively this granulation can be prepared by blending the copper antagonist(s) with lactose and dicalcium phosphate and wet granulating with the hydroxypropylcellulose solution in a Niro or Glatt high speed granulator and drying it in, for example, a Glatt fluid bed dryer.

This granulation is mixed with the disodium phosphate and magnesium stearate or other lubricant in a suitable blender, such as Patterson Kelly V-blender. The final blend is compressed into tablets on a suitable tablet machine, such as a Manesty beta-press.

Copper antagonists other than triethylenetetramine dihydrochloride or triethylenetetramine disuccinate may be used, as may statins other than simvastatin. Additionally, in certain cases, a copper antagonist compound which has already been considerably precomplexed with a non-copper metal ion as disclosed herein may be used, for example a triethylenetetramine precomplexed with calcium or another non-copper metal ion. Pentacoordinate copper antagonists may also be used, including for example, a triethylenetetramine complexed with calcium (or another non-copper metal) and another complexing agent, such as, for example, chloride, as disclosed herein.

Amounts of the copper antagonist(s) and statin(s), including the amounts of triethylenetetramine dihydrochloride or triethylenetetramine disuccinate and simvastatin set forth in this Example, may be varied, as appropriate. By way of example only, the amount of triethylenetetramine dihydrochloride or triethylenetetramine disuccinate (or other copper antagonist) may range from about 1 mg to about 750 mg (for example, 1 mg, 5 mg, 10 mg, 25 mg, 50 mg, 100 mg, 125 mg, 150 mg 200 mg, 250 mg, 400 mg, 500 mg, 600 mg, and 750 mg), and the amount of simvastatin may range from about 1 mg to about 40 mg (for example, 1 mg, 2 mg, 2.5 mg, 3 mg, 4 mg, 5 mg, 10 mg, 20 mg, 25 mg, 30 mg, and 40 mg). Other amounts may also be used. The amounts are not inflexible and may be determined, in part, for example, based on the number of tablets to be taken per day.

The tablet may be prepared for administration of drug, by way of example, in one or more doses, for example, one or two or more tablets once, twice, or more per day. Tablets are generally prepared for administration no more than four times per day, preferably less, and most preferably one or two times per day.

### EXAMPLE 6

### A Combination Capsule Employing Direct Filling

This Example describes preparation of capsules having a copper antagonist(s) such as, for example, one or more copper chelators (e.g., a trientine, such as triethylenetetramine dihydrochloride or triethylenetetramine disuccinate), and one or more 3-hydroxy-3-methylglutaryl coenzyme A reductase inhibitors, for example, statins (e.g., simvastatin), which may be prepared using compaction and direct compression methods.

Ingredients for capsules including, for example, triethylenetetramine dihydrochloride or triethylenetetramine disuccinate and simvastatin, are provided in the below table:

| | Exemplary | Mg per |
|---|---|---|
| Ingredient | Function | capsule |
| Triethylenetetramine | Copper | 100-300 |
| dihydrochloride or | binding | |
| triethylenetetramine | | |
| disuccinate | | |
| Simvastatin | HMG-CoA | 2-10 |
| | reductase | |
| | inhibitor | |
| Silicon dioxide | Glidant; | 1.56 |
| | desiccant | |
| Magnesium stearate | Lubricant | 1.56 |

The process employs compaction. The copper antagonist(s) is/are first compacted, for example, in a suitable roller compacter. Suitable roller compactors include, for example, the Vector Mini-Model TF (Vector Corp., Marion, Iowa). It is then milled with a Fitz mill (Fitzpatrick Company, Elmhurst, Ill.) or other suitable mill, such as a Quadro Comill, oscillating mill, or pin mill, for example.

The milled copper antagonist is blended with the statin, silicon dioxide and magnesium stearate or other lubricant in a suitable blender. Suitable blenders include, for example, v-Blenders (Patterson-Kelly), planetary blenders (Hobart Corp., Troy Ohio).

The final blend is filled into hard gelatin capsules with on a suitable encapsulation machine, such as a Zanasi 40 E capsule machine.

Copper antagonists other than triethylenetetramine dihydrochloride or triethylenetetramine disuccinate may be used, as may statins other than simvastatin. Additionally, in certain cases, a copper antagonist compound which has already been considerably precomplexed with a non-copper metal ion as disclosed herein may be used, for example a triethylenetetramine precomplexed with calcium or another non-copper metal ion. Pentacoordinate copper antagonists may also be used, including for example, a triethylenetetramine complexed with calcium (or another non-copper metal) and another complexing agent, such as, for example, chloride, as disclosed herein.

Amounts of the copper antagonist(s) and statin(s), including the amounts of triethylenetetramine dihydrochloride or triethylenetetramine disuccinate and simvastatin set forth in this Example, may be varied, as appropriate. By way of example only, the amount of triethylenetetramine dihydrochloride or triethylenetetramine disuccinate (or other copper antagonist) may range from about 1 mg to about 750 mg (for example, 1 mg, 5 mg, 10 mg, 25 mg, 50 mg, 100 mg, 125 mg, 150 mg 200 mg, 250 mg, 400 mg, 500 mg, 600 mg, and 750 mg), and the amount of simvastatin may range from about 1 mg to about 40 mg (for example, 1 mg, 2 mg, 2.5 mg, 3 mg, 4 mg, 5 mg, 10 mg, 20 mg, 25 mg, 30 mg, and 40 mg). Other amounts may also be used. The amounts are not inflexible and may be determined, in part, for example, based on the number of capsules to be taken per day.

A preferred capsule size is 300 mg. Thus, capsules may be prepared using appropriate doses within the ranges provided in order to yield a 300 mg size. The capsule may be prepared for administration of drug, by way of example, in one or more doses, for example, one or two or more tablets once, twice, or more per day. Capsules are generally prepared for administration no more than four times per day, preferably less, and most preferably one or two times per day.

### EXAMPLE 7

### A Combination Capsule Employing a Desiccant and Direct Filling

This Example describes preparation of capsules having a copper antagonist(s) such as, for example, one or more copper chelators (e.g., a trientine, such as triethylenetetramine dihydrochloride or triethylenetetramine disuccinate) and one or more 3-hydroxy-3-methylglutaryl coenzyme A reductase inhibitors, for example, statins (e.g., simvastatin), together with one or more desiccants, which may be prepared using compaction and direct compression methods.

Ingredients for capsules including, for example, triethylenetetramine dihydrochloride or triethylenetetramine disuccinate and simvastatin, are provided in the below table:

| | Exemplary | Mg per |
|---|---|---|
| Ingredient | Function | capsule |
| Triethylenetetramine | Copper | 100-300 |
| dihydrochloride or | binding | |
| triethylenetetramine | | |
| disuccinate | | |
| Simvastatin | HMG-CoA | 2-10 |
| | reductase | |
| | inhibitor | |
| Disodium phosphate | Desiccant | 16.49 |
| anhydrous | | |
| Silicon dioxide | Glidant; | 1.65 |
| | desiccant | |
| Magnesium stearate | Lubricant | 1.65 |

The process employs compaction, blending and encapsulation. The copper antagonist(s) is/are first compacted, for example, in a suitable roller compacter. Suitable roller compactors include, for example, the Vector Mini-Model TF (Vector Corp., Marion, Iowa). It is then milled with a Fitz mill (Fitzpatrick Company, Elmhurst, Ill.) or other suitable mill, such as a Quadro Comill, oscillating mill, or pin mill, for example.

The milled copper antagonist is blended with the statin, disodium phosphate, silicon dioxide and magnesium stearate or other lubricant in a suitable blender. Suitable blenders include, for example, V-blenders (Patterson-Kelly), planetary blenders (Hobart Corp., Troy Ohio).

The final blend is encapsulated into hard gelatin capsules on a suitable capsule machine, such as a Zanasi 40E capsule machine.

Copper antagonists other than triethylenetetramine dihydrochloride or triethylenetetramine disuccinate may be used, as may statins other than simvastatin. Additionally, in certain cases, a copper antagonist compound which has already been considerably precomplexed with a non-copper metal ion as disclosed herein may be used, for example a triethylenetetramine precomplexed with calcium or another non-copper metal ion. Pentacoordinate copper antagonists may also be used, including for example, a triethylenetetramine complexed with calcium (or another non-copper metal) and another complexing agent, such as, for example, chloride, as disclosed herein. Amounts of the copper antagonist(s) and statin(s), including the amounts of triethylenetetramine dihydrochloride or triethylenetetramine disuccinate and simvastatin set forth in this Example, may be varied, as appropriate. By way of example only, the amount of triethylenetetramine dihydrochloride or triethylenetetramine disuccinate (or other copper antagonist) may range from about 1 mg to about 750 mg (for example, 1 mg, 5 mg, 10 mg, 25 mg, 50 mg, 100 mg, 125 mg, 150 mg 200 mg, 250 mg, 400 mg, 500 mg, 600 mg, and 750 mg), and the amount of simvastatin may range from about 1 mg to about 40 mg (for example, 1 mg, 2 mg, 2.5 mg, 3 mg, 4 mg, 5 mg, 10 mg, 20 mg, 25 mg, 30 mg, and 40 mg). Other amounts may also be used. The amounts are not inflexible and may be determined, in part, for example, based on the number of capsules to be taken per day.

A preferred capsule size is 300 mg. Thus, capsules may be prepared using appropriate doses within the ranges provided in order to yield a 300 mg size. The capsule may be prepared for administration of drug, by way of example, in one or more doses, for example, one or two or more tablets once, twice, or more per day. Capsules are generally prepared for administration no more than four times per day, preferably less, and most preferably one or two times per day.

### EXAMPLE 8

### A Combination Capsule Employing Fillers and Direct Filling

This Example describes preparation of capsules having a copper antagonist(s) such as, for example, one or more copper chelators (e.g., a trientine, such as triethylenetetramine dihydrochioride or triethylenetetramine disuccinate) and one or more 3-hydroxy-3-methylglutaryl coenzyme A reductase inhibitors, for example, statins (e.g., simvastatin), together with one or more fillers, which may be prepared using direct filling methods.

Ingredients for capsules including, for example, triethylenetetramine dihydrochloride or triethylenetetramine disuccinate and simvastatin, are provided in the below table:

| | Exemplary | Mg per |
|---|---|---|
| Ingredient | Function | capsule |
| Triethylenetetramine | Copper | 100-300 |
| dihydrochloride or | binding | |
| triethylenetetramine | | |
| disuccinate | | |
| Simvastatin | HMG-CoA | 2-10 |
| | reductase | |
| | inhibitor | |
| Lactose USP | Filler | 80.00 |
| Crosscarmellose | Disintegrant | 8.00 |
| sodium | | |
| Magnesium stearate | Lubricant | 2.00 |

The process employs a combination of blending and direct encapsulation techniques. The copper antagonist is blended, for example, with the statin and lactose in a suitable blender. Suitable blenders include, for example, v-blenders (Patterson-Kelly), and planetary blenders (Hobart Corp).

The resulting blend is mixed with the crosscarmellose sodium in the same blender. This blend may be milled and screened in a Fitz mill (Fitzpatrick Corp) or other suitable mill, such as a Quadro Comill, oscillating mill, or pin mill, for example.

The resulting blend is mixed with the magnesium stearate, or other lubricant, which may also be accomplished in a suitable blender.

The final blend is filled into hard gelatin capsules on a suitable encapsulation machine, such as a Zanasi capsule machine.

Copper antagonists other than triethylenetetramine dihydrochloride or triethylenetetramine disuccinate may be used, as may statins other than simvastatin. Additionally, in certain cases, a copper antagonist compound which has already been considerably precomplexed with a non-copper metal ion as disclosed herein may be used, for example a triethylenetetramine precomplexed with calcium or another non-copper metal ion. Pentacoordinate copper antagonists may also be used, including for example, a triethylenetetramine complexed with calcium (or another non-copper metal) and another complexing agent, such as, for example, chloride, as disclosed herein.

Amounts of the copper antagonist(s) and statin(s), including the amounts of triethylenetetramine dihydrochloride or triethylenetetramine disuccinate and simvastatin set forth in this Example, may be varied, as appropriate. By way of example only, the amount of triethylenetetramine dihydrochloride or triethylenetetramine disuccinate (or other copper antagonist) may range from about 1 mg to about 750 mg (for example, 1 mg, 5 mg, 10 mg, 25 mg, 50 mg, 100 mg, 125 mg, 150 mg 200 mg, 250 mg, 400 mg, 500 mg, 600 mg, and 750 mg), and the amount of simvastatin may range from about 1 mg to about 40 mg (for example, 1 mg, 2 mg, 2.5 mg, 3 mg, 4 mg, 5 mg, 10 mg, 20 mg, 25 mg, 30 mg, and 40 mg). Other amounts may also be used. The amounts are not inflexible and may be determined, in part, for example, based on the number of capsules to be taken per day.

A preferred capsule size is 300 mg. Thus, capsules may be prepared using appropriate doses within the ranges provided in order to yield a 300 mg size. The capsule may be prepared for administration of drug, by way of example, in one or more doses, for example, one or two or more tablets once, twice, or more per day. Capsules are generally prepared for. administration no more than four times per day, preferably less, and most preferably one or two times per day.

### EXAMPLE 9

### A Combination Capsule Employing Wet Granulation

This Example describes preparation of capsules having a copper antagonist(s) such as, for example, one or more copper chelators (e.g., a trientine, such as triethylenetetramine dihydrochloride or triethylenetetramine disuccinate) and one or more 3-hydroxy-3-methylglutaryl coenzyme A reductase inhibitors, for example, statins (e.g., simvastatin), which may be prepared using wet granulation methods.

Ingredients for capsules including, for example, triethylenetetramine dihydrochloride or triethylenetetramine disuccinate and simvastatin, are provided in the below table:

| | Exemplary | Mg per |
|---|---|---|
| Ingredient | Function | capsule |
| Triethylenetetramine | Copper | 100-300 |
| dihydrochloride or | binding | |
| triethylenetetramine | | |
| disuccinate | | |
| Simvastatin | HMG-CoA | 2-10 |
| | reductase | |
| | inhibitor | |
| Lactose USP | Filler | 36.69 |
| Sodium starch glycollate | Disintegrant | 7.34 |
| Magnesium stearate | Lubricant | 1.83 |
| Hydroxypropylcellulose | Granulation | 11.00 |
| | binder | |

This tablet is prepared using wet granulation methods. The copper antagonist(s) is blended with the statin(s), and lactose and in a suitable fluid bed granulator/dryer. Suitable granulator/dryers include Glatt or Niro fluid bed granulator/dryers. The hydroxypropylcellulose is dissolved in water or ethanol, and the blend is wet granulated with the solution of hydroxypropylcellulose in a suitable granulator/dryer. The wet granulation is dried in the granulator dryer. Where triethylenetetramine dihydrochloride is included in this formulation it is preferably precomplexed with a non-copper metal ion as disclosed herein, e.g., calcium, to enhance stability.

Alternatively this granulation can be prepared by blending the copper antagonist(s) with lactose and wet granulating with the hydroxypropylcellulose solution in a Niro or Glatt high speed granulator and drying it in a Glatt fluid bed dryer.

This granulation is mixed with the sodium starch glycolate and magnesium stearate in a suitable blender, such as Patterson Kelly V-blender. The final blend is compressed into tablets on a suitable tablet machine, such as a Manesty beta-press.

Copper antagonists other than triethylenetetramine dihydrochloride or triethylenetetramine disuccinate may be used, as may statins other than simvastatin. Additionally, in certain cases, a copper antagonist compound which has already been considerably precomplexed with a non-copper metal ion as disclosed herein may be used, for example a triethylenetetramine precomplexed with calcium or another non-copper metal ion. Pentacoordinate copper antagonists may also be used, including for example, a triethylenetetramine complexed with calcium (or another non-copper metal) and another complexing agent, such as, for example, chloride, as disclosed herein.

Amounts of the copper antagonist(s) and statin(s), including the amounts of triethylenetetramine dihydrochloride or triethylenetetramine disuccinate and simvastatin set forth in this Example, may be varied, as appropriate. By way of example only, the amount of triethylenetetramine dihydrochloride or triethylenetetramine disuccinate (or other copper antagonist) may range from about 1 mg to about 750 mg (for example, 1 mg, 5 mg, 10 mg, 25 mg, 50 mg, 100 mg, 125 mg, 150 mg 200 mg, 250 mg, 400 mg, 500 mg, 600 mg, and 750 mg), and the amount of simvastatin may range from about 1 mg to about 40 mg (for example, 1 mg, 2 mg, 2.5 mg, 3 mg, 4 mg, 5 mg, 10 mg, 20 mg, 25 mg, 30 mg, and 40 mg). Other amounts may also be used. The amounts are not inflexible and may be determined, in part, for example, based on the number of capsules to be taken per day.

A preferred capsule size is 300 mg. Thus, capsules may be prepared using appropriate doses within the ranges provided in order to yield a 300 mg size. The capsule may be prepared for administration of drug, by way of example, in one or more doses, for example, one or two or more tablets once, twice, or more per day. Capsules are generally prepared for administration no more than four times per day, preferably less, and most preferably one or two times per day.

### EXAMPLE 10

### Combination Capsule Employing a Desiccant and Wet Granulation

This Example describes preparation of capsules having a copper antagonist(s) such as, for example, one or more copper chelators (e.g., a trientine, such as triethylenetetramine dihydrochloride or triethylenetetramine disuccinate) and one or more 3-hydroxy-3-methylglutaryl coenzyme A reductase inhibitors, for example, statins (e.g., simvastatin), together with one or more desiccants, which may be prepared using wet granulation methods.

Ingredients for capsules including, for example, triethylenetetramine dihydrochloride or triethylenetetramine disuccinate and simvastatin, are provided in the below table:

| | Exemplary | Mg per |
|---|---|---|
| Ingredient | Function | capsule |
| Triethylenetetramine | Copper | 300 |
| dihydrochloride or | binding | |
| triethylenetetramine | | |
| disuccinate | | |
| Simvastatin | HMG-CoA | 10 |
| | reductase | |
| | inhibitor | |
| Lactose | Filler | 87.94 |
| Disodium phosphate | Desiccant | 21.99 |
| anhydrous | | |
| Crosscarmellose | Disintegrant | 4.40 |
| sodium | | |
| Magnesium stearate | Lubricant | 2.20 |
| Hydroxypropylcellulose | Granulation | 13.19 |
| | binder | |

This tablet is prepared using wet granulation methods. The copper antagonist(s) is blended with the statin(s) and lactose in a suitable fluid bed granulator/dryer. Suitable granulator/dryers include Glatt or Niro fluid bed granulator/dryers. The hydroxypropylcellulose is dissolved in water or ethanol, and the blend is wet granulated with the solution of hydroxypropylcellulose in a suitable granulator/dryer. The wet granulation is dried in the granulator dryer. Where triethylenetetramine dihydrochloride is included in this formulation it is preferably precomplexed with a non-copper metal ion as disclosed herein, e.g., calcium, to enhance stability.

Alternatively this granulation can be prepared by blending the copper antagonist(s) with lactose and dicalcium phosphate and wet granulating with the hydroxypropylcellulose solution in a Niro or Glatt high speed granulator and drying it in a Glatt fluid bed dryer.

This granulation is mixed with the disodium phosphate, crosscarmellose sodium and magnesium stearate in a suitable blender, such as Patterson Kelly V-blender. The final blend is filled into hard gelatin capsules in a suitable encapsulation machine, such as a Zanasi 40E capsule machine.

Copper antagonists other than triethylenetetramine dihydrochloride or triethylenetetramine disuccinate may be used, as may statins other than simvastatin. Additionally, in certain cases, a copper antagonist compound which has already been considerably precomplexed with a non-copper metal ion as disclosed herein may be used, for example a triethylenetetramine precomplexed with calcium or another non-copper metal ion. Pentacoordinate copper antagonists may also be used, including for example, a triethylenetetramine complexed with calcium (or another non-copper metal) and another complexing agent, such as, for example, chloride, as disclosed herein. Amounts of the copper antagonist(s) and statin(s), including the amounts of triethylenetetramine dihydrochloride or triethylenetetramine disuccinate and simvastatin set forth in this Example, may be varied, as appropriate. By way of example only, the amount of triethylenetetramine dihydrochloride or triethylenetetramine disuccinate (or other copper antagonist) may range from about 1 mg to about 750 mg (for example, 1 mg, 5 mg, 10 mg, 25 mg, 50 mg, 100 mg, 125 mg, 150 mg 200 mg, 250 mg, 400 mg, 500 mg, 600 mg, and 750 mg), and the amount of simvastatin may range from about 1 mg to about 40 mg (for example, 1 mg, 2 mg, 2.5 mg, 3 mg, 4 mg, 5 mg, 10 mg, 20 mg, 25 mg, 30 mg, and 40 mg). Other amounts may also be used. The amounts are not inflexible and may be determined, in part, for example, based on the number of capsules to be taken per day.

A preferred capsule size is 300 mg. Thus, capsules may be prepared using appropriate doses within the ranges provided in order to yield a 300 mg size. The capsule may be prepared for administration of drug, by way of example, in one or more doses, for example, one or two or more tablets once, twice, or more per day. Capsules are generally prepared for administration no more than four times per day, preferably less, and most preferably one or two times per day.

### EXAMPLE 11

### A Controlled Release Combination Tablet

This Example describes preparation of matrix controlled release tablets having a copper antagonist(s) such as, for example, one or more copper chelators (e.g., a trientine, such as triethylenetetramine dihydrochloride or triethylenetetramine disuccinate), and one or more 3-hydroxy-3-methylglutaryl coenzyme A reductase inhibitors, for example, statins (e.g., simvastatin), which may be prepared using roller compaction and direct compression methods.

Ingredients for tablets including, for example, triethylenetetramine dihydrochloride or triethylenetetramine disuccinate and simvastatin, are provided in the below table:

| | Exemplary | Mg per | | | Kg per |
|---|---|---|---|---|---|
| Ingredient | Function | tablet | | % | 10 Kg |
| Triethylenetetramine | Copper | 300 | mg | 67.26 | 6.726 |
| dihydrochloride or | binding | | | | |
| triethylenetetramine | | | | | |
| disuccinate | | | | | |
| Simvastatin | HMG- | 10 | | 2.24 | 0.224 |
| | CoA | | | | |
| | reductase | | | | |
| | inhibitor | | | | |
| Hydroxypropylmethyl- | Controlled | 89.21 | | 20.00 | 2.00 |
| cellulose | release | | | | |
| | agent | | | | |
| Lactose | Filler | 44.60 | | 10.00 | 1.00 |
| Magnesium stearate | Lubricant | 2.23 | | 0.50 | 0.050 |
| Total | | 446.04 | | 100 | 10 |

The process employs compaction and direct compression. The copper antagonist(s) is/are first compacted, for example, in a suitable roller compacter. Suitable roller compactors include, for example, the Vector Mini-Model TF (Vector Corp., Marion, Iowa). It is then milled with a Fitz mill (Fitzpatrick Company, Elmhurst, Ill.) or other suitable mill, such as a Comill mill, oscillator mill, or pin mill, for example.

The milled copper antagonist is blended with statin, hydroxypropyl-methylcellulose, and lactose in a suitable blender. Suitable blenders include V-Blenders (Patterson-Kelly, and planetary blenders (Hobart Corp., Troy Ohio).

This blend is blended with the magnesium stearate or other lubricant in the same blender.

The final blend is compressed into tablets using a suitable tablet machine, such as a Manesty beta-press (Manesty, Knowsley, Merseyside, UK).

Copper antagonists other than triethylenetetramine dihydrochloride or triethylenetetramine disuccinate may be used, as may statins other than simvastatin. Additionally, in certain cases, a copper antagonist compound which has already been considerably precomplexed with a non-copper metal ion as disclosed herein may be used, for example a triethylenetetramine precomplexed with calcium or another non-copper metal ion. Pentacoordinate copper antagonists may also be used, including for example, a triethylenetetramine complexed with calcium (or another non-copper metal) and another complexing agent, such as, for example, chloride, as disclosed herein.

Amounts of the copper antagonist(s) and statin(s), including the amounts of triethylenetetramine dihydrochloride or triethylenetetramine disuccinate and simvastatin set forth in this Example, may be varied, as appropriate. By way of example only, the amount of triethylenetetramine dihydrochloride or triethylenetetramine disuccinate (or other copper antagonist) may range from about 1 mg to about 750 mg (for example, 1 mg, 5 mg, 10 mg, 25 mg, 50 mg, 100 mg, 125 mg, 150 mg 200 mg, 250 mg, 400 mg, 500 mg, 600 mg, and 750 mg), and the amount of simvastatin may range from about 1 mg to about 40 mg (for example, 1 mg, 2 mg, 2.5 mg, 3 mg, 4 mg, 5 mg, 10 mg, 20 mg, 25 mg, 30 mg, and 40 mg). Other amounts may also be used. The amounts are not inflexible and may be determined, in part, for example, based on the number of tablets to be taken per day.

The tablet may be prepared for administration of drug, by way of example, in one or more doses, for example, one or two or more tablets once, twice, or more per day. Tablets are generally prepared for administration no more than four times per day, preferably less, and most preferably one or two times per day.

### EXAMPLE 12

### A Combination Capsule Containing Enteric Coated Beads

This Example describes preparation of capsules containing enteric beads having a copper antagonist(s) such as, for example, one or more copper chelators (e.g., a trientine, such as triethylenetetramine dihydrochloride or triethylenetetramine disuccinate), and one or more 3-hydroxy-3-methylglutaryl coenzyme A reductase inhibitors, for example, statins (e.g., simvastatin), which may be prepared using granulation, spheronization, and bead coating methods.

Ingredients for capsules including, for example, triethylenetetramine dihydrochloride or triethylenetetramine disuccinate and simvastatin, are provided in the below table:

| | Exemplary | Mg per |
|---|---|---|
| Ingredient | Function | capsule |
| Triethylenetetramine | Copper | 300 |
| dihydrochloride or | binding | |
| triethylenetetramine | | |
| disuccinate | | |
| Simvastatin | HMG-CoA | 10 |
| | reductase | |
| | inhibitor | |
| Hydroxypropylcellulose | Granulation | 10.70 |
| | binder | |
| Cellulose acetate | Delayed | 35.67 |
| phthalate | release agent | |
| Talc | Anti adherent | 0.36 |

The copper antagonist(s), statin(s), and hydroxypropylcellulose are blended in a suitable granulator-spheronizer, such as a Niro Roto-Processor spheronizer. Water or alcohol is used to wet the granulation and the wet mass is spheronized to beads on the processor. The beads are dried in a fluid bed coating/drying processor, such as a Niro Precision coater.

A commercial aqueous or alcohol solution of cellulose acetate phthalate, for example, Aquacote CPD-FMC Corporation, is used to coat the beads in the coating-drying processor. The dried beads are coated with the solution and dried in fluid bed coating apparatus; Talc can be added to keep the beads free flowing. The beads are filled into hard gelatin capsules using an appropriate capsule-filling machine, such as a Zanasi encapsulation machine

Copper antagonists other than triethylenetetramine dihydrochloride or triethylenetetramine disuccinate may be used, as may statins other than simvastatin. Additionally, in certain cases, a copper antagonist compound which has already been considerably precomplexed with a non-copper metal ion as disclosed herein may be used, for example a triethylenetetramine precomplexed with calcium or another non-copper metal ion. Pentacoordinate copper antagonists may also be used, including for example, a triethylenetetramine complexed with calcium (or another non-copper metal) and another complexing agent, such as, for example, chloride, as disclosed herein.

Amounts of the copper antagonist(s) and statin(s), including the amounts of triethylenetetramine dihydrochloride or triethylenetetramine disuccinate and simvastatin set forth in this Example, may be varied, as appropriate. By way of example only, the amount of triethylenetetramine dihydrochloride or triethylenetetramine disuccinate (or other copper antagonist) may range from about 1 mg to about 750 mg (for example, 1 mg, 5 mg, 10 mg, 25 mg, 50 mg, 100 mg, 125 mg, 150 mg 200 mg, 250 mg, 400 mg, 500 mg, 600 mg, and 750 mg), and the amount of simvastatin may range from about 1 mg to about 40 mg (for example, 1 mg, 2 mg, 2.5 mg, 3 mg, 4 mg, 5 mg, 10 mg, 20 mg, 25 mg, 30 mg, and 40 mg). Other amounts may also be used. The amounts are not inflexible and may be determined, in part, for example, based on the number of capsules to be taken per day.

A preferred capsule size is 300 mg. Thus, capsules may be prepared using appropriate doses within the ranges provided in order to yield a 300 mg size. The capsule may be prepared for administration of drug, by way of example, in one or more doses, for example, one or two or more tablets once, twice, or more per day. Capsules are generally prepared for administration no more than four times per day, preferably less, and most preferably one or two times per day.

### EXAMPLE 13

### Combination Tablet

This Example describes preparation of tablets having a copper antagonist(s) such as, for example, one or more copper chelators (e.g., a trientine, such as triethylenetetramine dihydrochloride or triethylenetetramine disuccinate), and one or more 3-hydroxy-3-methylglutaryl coenzyme A reductase inhibitors- for example, statins (e.g., atorvastatin), which may be prepared using compaction and direct compression methods.

Ingredients for tablets including, for example, triethylenetetramine dihydrochloride or triethylenetetramine disuccinate and atorvastatin, are provided in the below table:

| | Exemplary | Mg per | | Kg per | |
|---|---|---|---|---|---|
| Ingredient | Function | tablet | % | 10 kg | |
| Triethylenetetramine | Copper | 300 | 95.81 | 9.581 | |
| dihydrochloride or | binding | | | | |
| triethylenetetramine | | | | | |
| disuccinate | | | | | |
| Atorvastatin | HMG-CoA | 10 | 3.19 | 0.319 | |
| | reductase | | | | |
| | inhibitor | | | | |
| Silicon dioxide | Glidant; | 1.57 | 0.50 | 0.05 | |
| | desiccant | | | | |
| Magnesium stearate | Lubricant | 1.57 | 0.50 | 0.05 | |
| Total | | 313.14 | 100 | 10 | Kg |

The process employs compaction and direct compression. The copper antagonist(s) is/are first compacted, for example, in a suitable roller compacter. Suitable roller compactors include, for example, the Vector Mini-Model TF (Vector Corp., Marion, Iowa). It is then milled with a Fitz mill (Fitzpatrick Company, Elmhurst, Ill.) or other suitable mill, such as a Quadro Comill, oscillator mill, or pin mill, for example.

The milled copper antagonist is blended with the statin, silicon dioxide and magnesium stearate, for example, in a suitable blender. Suitable blenders include v-Blenders (Patterson-Kelly), planetary blenders (Hobart Corp., Troy Ohio).

The final blend is compressed into tablets using a suitable tablet machine, such as a Manesty beta-press (Manesty, Knowsley, Merseyside, UK).

Copper antagonists other than triethylenetetramine dihydrochloride or triethylenetetramine disuccinate may be used, as may statins other than atorvastatin. Additionally, in certain cases, a copper antagonist compound which has already been considerably precomplexed with a non-copper metal ion as disclosed herein may be used, for example a triethylenetetramine precomplexed with calcium or another non-copper metal ion. Pentacoordinate copper antagonists may also be used, including for example, a triethylenetetramine complexed with calcium (or another non-copper metal) and another complexing agent, such as, for example, chloride, as disclosed herein.

Amounts of the copper antagonist(s) and statin(s), including the amounts of triethylenetetramine dihydrochloride or triethylenetetramine disuccinate and atorvastatin set forth in this Example, may be varied, as appropriate. By way of example only, the amount of triethylenetetramine dihydrochloride or triethylenetetramine disuccinate (or other copper antagonist) may range from about 1 mg to about 750 mg (for example, 1 mg, 5 mg, 10 mg, 25 mg, 50 mg, 100 mg, 125 mg, 150 mg 200 mg, 250 mg, 400 mg, 500 mg, 600 mg, and 750 mg), and the amount of atorvastatin may range from about 1 mg to about 40 mg (for example, 1 mg, 2 mg, 2.5 mg, 3 mg, 4 mg, 5 mg, 10 mg, 20 mg, 25 mg, 30 mg, 40 mg, and 80 mg). Other amounts may also be used. The amounts are not inflexible and may be determined, in part, for example, based on the number of tablets to be taken per day.

The tablet may be prepared for administration of drug, by way of example, in one or more doses, for example, one or two or more tablets once, twice, or more per day. Tablets are generally prepared for administration no more than four times per day, preferably less, and most preferably one or two times per day.

### EXAMPLE 14

### Combination Tablet with Fillers

This Example describes preparation of tablets including fillers having a copper antagonist(s) such as, for example, one or more copper chelators (e.g., a trientine, such as triethylenetetramine dihydrochloride or triethylenetetramine disuccinate), and one or more 3-hydroxy-3-methylglutaryl coenzyme A reductase inhibitors, for example, statins (e.g., atorvastatin), which may be prepared using blending and direct compression methods.

Ingredients for tablets including, for example, triethylenetetramine dihydrochloride or triethylenetetramine disuccinate and atorvastatin, are provided in the below table:

| | | (ii) Mg | | |
|---|---|---|---|---|
| | Exemplary | per | | Kg per |
| Ingredient | Function | tablet | % | 10 Kg |
| Triethylenetetramine | Copper | 300 | 74.52 | 7.452 |
| dihydrochloride or | binding | | | |
| triethylenetetramine | | | | |
| disuccinate | | | | |
| Atorvastatin | HMG-CoA | 10 | 2.48 | 0.248 |
| | reductase | | | |
| | inhibitor | | | |
| Microcrystalline | Filler-binder | 80.52 | 20.00 | 2.00 |
| cellulose | | | | |
| Croscarmellose | Disintegrant | 8.05 | 2.00 | 0.20 |
| sodium | | | | |
| Silicon dioxide | Glidant; | 2.01 | 0.50 | 0.05 |
| | desiccant | | | |
| Magnesium stearate | Lubricant | 2.01 | 0.50 | 0.05 |
| Total | | 402.59 | 100 | 10 |

The process employs a combination of blending and direct compression techniques. The copper antagonist is blended, for example, with the statin in a suitable blender. Suitable blenders include, for example, V-Blenders (Patterson-Kelly), planetary blenders (Hobart Corp).

The resulting blend is mixed with microcrystalline cellulose, which may also be done in a suitable blender. This blend is milled and screened in a Fitz mill (Fitzpatrick Corp) or other suitable mill, such as a Quadro Comill, oscillating mill, or pin mill, for example.

The resulting blend is mixed with the silicon dioxide and magnesium stearate, which may also be accomplished in a suitable blender.

The final blend is compressed into tablets on a suitable tablet machine, such as a Manesty beta-press.

Copper antagonists other than triethylenetetramine dihydrochloride or triethylenetetramine disuccinate may be used, as may statins other than atorvastatin. Additionally, in certain cases, a copper antagonist compound which has already been considerably precomplexed with a non-copper metal ion as disclosed herein may be used, for example a triethylenetetramine precomplexed with calcium or another non-copper metal ion. Pentacoordinate copper antagonists may also be used, including for example, a triethylenetetramine complexed with calcium (or another non-copper metal) and another complexing agent, such as, for example, chloride, as disclosed herein.

Amounts of the copper antagonist(s) and statin(s), including the amounts of triethylenetetramine dihydrochloride or triethylenetetramine disuccinate and atorvastatin set forth in this Example, may be varied, as appropriate. By way of example only, the amount of triethylenetetramine dihydrochloride or triethylenetetramine disuccinate (or other copper antagonist) may range from about 1 mg to about 750 mg (for example, 1 mg, 5 mg, 10 mg, 25 mg, 50 mg, 100 mg, 125 mg, 150 mg 200 mg, 250 mg, 400.mg, 500 mg, 600 mg, and 750 mg), and the amount of atorvastatin may range from about 1 mg to about 40 mg (for example, 1 mg, 2 mg, 2.5 mg, 3 mg, 4 mg, 5 mg, 10 mg, 20 mg, 25 mg, 30 mg, 40 mg, and 80 mg). Other amounts may also be used. The amounts are not inflexible and may be determined, in part, for example, based on the number of tablets to be taken per day.

The tablet may be prepared for administration of drug, by way of example, in one or more doses, for example, one or two or more tablets once, twice, or more per day. Tablets are generally prepared for administration no more than four times per day, preferably less, and most preferably one or two times per day.

### EXAMPLE 15

### Combination Tablet with Desiccant(s)

This Example describes preparation of tablets having a copper antagonist(s) such as, for example, one or more copper chelators (e.g;., a trientine, such as triethylenetetramine dihydrochloride or triethylenetetramine disuccinate) and one or more 3-hydroxy-3-methylglutaryl coenzyme A reductase inhibitors, for example, statins (e.g., atorvastatin), together with one or more desiccants, which may be prepared using direct compression methods.

Ingredients for tablets including, for example, triethylenetetramine dihydrochloride or triethylenetetramine disuccinate and atorvastatin, are provided in the below table:

| | Exemplary | Mg per | | Kg per |
|---|---|---|---|---|
| Ingredient | Function | tablet | % | 10 Kg |
| Triethylenetetramine | Copper | 300 | 90.97 | 9.097 |
| dihydrochloride or | binding | | | |
| triethylenetetramine | | | | |
| disuccinate | | | | |
| Atorvastatin | HMG-CoA | 10 | 3.03 | 0.303 |
| | reductase | | | |
| | inhibitor | | | |
| Disodium phosphate | Desiccant | 16.49 | 5.0 | 0.50 |
| anhydrous | | | | |
| Silicon dioxide | Glidant; | 1.65 | 0.5 | 0.05 |
| | desiccant | | | |
| Magnesium stearate | Lubricant | 1.65 | 0.5 | 0.05 |
| Total | | 329.79 | 100 | 10 |

The process employs compaction, blending and direct compression. The copper antagonist(s), statin(s) and desiccant(s) are blended in a suitable blender. Suitable blenders include, for example, v-blenders (Patterson-Kelly), planetary blenders (Hobart). This blend is compacted in a suitable roller compacter, such as a Vector Mini-model TF. It is then milled and screened in a Fitz mill or other suitable mill. Suitable mills include a Quadro Comill, oscillating mills, and pin mills, for example.

The resulting blend is blended with the atorvastatin, silicon dioxide and magnesium stearate in a suitable blender.

The final blend is compacted into tablets on a suitable tablet machine, such as a Manesty beta press.

Copper antagonists other than triethylenetetramine dihydrochloride or triethylenetetramine disuccinate may be used, as may statins other than atorvastatin. Additionally, in certain cases, a copper antagonist compound which has already been considerably precomplexed with a non-copper metal ion as disclosed herein may be used, for example a triethylenetetramine precomplexed with calcium or another non-copper metal ion. Pentacoordinate copper antagonists may also be used, including for example, a triethylenetetramine complexed with calcium (or another non-copper metal) and another complexing agent, such as, for example, chloride, as disclosed herein.

Amounts of the copper antagonist(s) and statin(s), including the amounts of triethylenetetramine dihydrochloride or triethylenetetramine disuccinate and atorvastatin set forth in this Example, may be varied, as appropriate. By way of example only, the amount of triethylenetetramine dihydrochloride or triethylenetetramine disuccinate (or other copper antagonist) may range from about 1 mg to about 750 mg (for example, 1 mg, 5 mg, 10 mg, 25 mg, 50 mg, 100 mg, 125 mg, 150 mg 200 mg, 250 mg, 400 mg, 500 mg, 600 mg, and 750 mg), and the amount of atorvastatin may range from about 1 mg to about 40 mg (for example, 1 mg, 2 mg, 2.5 mg, 3 mg, 4 mg, 5 mg, 10 mg, 20 mg, 25 mg, 30 mg, 40 mg, and 80 mg). Other amounts may also be used. The amounts are not inflexible and may be determined, in part, for example, based on the number of tablets to be taken per day.

The tablet may be prepared for administration of drug, by way of example, in one or more doses, for example, one or two or more tablets once, twice, or more per day. Tablets are generally prepared for administration no more than four times per day, preferably. less, and most preferably one or two times per day.

### EXAMPLE 16

### Combination Tablet with Wet Granulation Binder(s)

This Example describes preparation of tablets having a copper antagonist(s) such as, for example, one or more copper chelators (e.g., a trientine, such as triethylenetetramine dihydrochloride or triethylenetetramine disuccinate) and one or more 3-hydroxy-3-methylglutaryl coenzyme A reductase inhibitors, for example, statins (e.g., atorvastatin), which may be prepared using wet granulation methods.

Ingredients for tablets including, for example, triethylenetetramine dihydrochloride or triethylenetetramine disuccinate and atorvastatin, are provided in the below table:

| | Exemplary | Mg per | | Kg per |
|---|---|---|---|---|
| Ingredient | Function | Tablet | % | 10 Kg |
| Triethylenetetramine | Copper | 300 | 72.10 | 7.21 |
| dihydrochloride or | binding | | | |
| triethylenetetramine | | | | |
| disuccinate | | | | |
| Atorvastatin | HMG-CoA | 10 | 2.40 | 0.240 |
| | reductase | | | |
| | inhibitor | | | |
| Lactose USP | Filler | 41.61 | 10.00 | 1.00 |
| Dicalcium phosphate | Filler | 41.61 | 10.00 | 1.00 |
| Crosscarmellose | Disintegrant | 8.32 | 2.00 | 0.20 |
| sodium | | | | |
| Hydroxypropylcellulose | Granulation | 12.48 | 3.00 | 0.30 |
| | binder | | | |
| Magnesium stearate | Lubricant | 2.06 | 0.50 | 0.05 |
| Total | | 416.08 | 100 | 10.0 |

This tablet is prepared using wet granulation methods. The copper antagonist(s) is blended with the statin(s), lactose and dicalcium phosphate in a suitable fluid bed granulator/dryer. Suitable granulator/dryers include Glatt or Niro fluid bed granulator/dryers. The hydroxypropylcellulose is dissolved in water or ethanol, and the blend is wet granulated with the solution of hydroxypropylcellulose in the suitable granulator/dryer. The wet granulation is dried in the granulator dryer. Where triethylenetetramine dihydrochloride is included in this formulation it is preferably precomplexed with a non-copper metal ion as disclosed herein, e.g., calcium, to enhance stability.

Alternatively this granulation can be prepared by blending the copper antagonist(s) with lactose and dicalcium phosphate and wet granulating with the hydroxypropylcellulose solution in a Niro or Glatt high speed granulator and drying it in a Glatt fluid bed dryer.

This final granulation is mixed with the crosscarmellose sodium and magnesium stearate or other lubricant in a suitable blender, such as Patterson Kelly V-blender.

The final blend is compressed into tablets on a suitable tablet machine, such as a Manesty beta-press.

Copper antagonists other than triethylenetetramine dihydrochloride or triethylenetetramine disuccinate may be used, as may statins other than atorvastatin. Additionally, in certain cases, a copper antagonist compound which has already been considerably precomplexed with a non-copper metal ion as disclosed herein may be used, for example a triethylenetetramine precomplexed with calcium or another non-copper metal ion. Pentacoordinate copper antagonists may also be used, including for example, a triethylenetetramine complexed with calcium (or another non-copper metal) and another complexing agent, such as, for example, chloride, as disclosed herein.

Amounts of the copper antagonist(s) and statin(s), including the amounts of triethylenetetramine dihydrochloride or triethylenetetramine disuccinate and atorvastatin set forth in this Example, may be varied, as appropriate. By way of example only, the amount of triethylenetetramine dihydrochloride or triethylenetetramine disuccinate (or other copper antagonist) may range from about 1 mg to about 750 mg (for example, 1 mg, 5 mg, 10 mg, 25 mg, 50 mg, 100 mg, 125 mg, 150 mg 200 mg, 250 mg, 400 mg, 500 mg, 600 mg, and 750 mg), and the amount of atorvastatin may range from about 1 mg to about 40 mg (for example, 1 mg, 2 mg, 2.5 mg, 3 mg, 4 mg, 5 mg, 10 mg, 20 mg, 25 mg, 30 mg, 40 mg, and 80 mg). Other amounts may also be used. The amounts are not inflexible and may be determined, in part, for example, based on the number of tablets to be taken per day.

The tablet may be prepared for administration of drug, by way of example, in one or more doses, for example, one or two or more tablets once, twice, or more per day. Tablets are generally prepared for administration no more than four times per day, preferably less, and most preferably one or two times per day.

### EXAMPLE 17

### A Combination Tablet Employing a Wet Granulation and a Desiccant

This Example describes preparation of tablets having a copper antagonist(s) such as, for example, one or more copper chelators (e.g., a trientine, such as triethylenetetramine dihydrochloride or triethylenetetramine disuccinate) and one or more 3-hydroxy-3-methylglutaryl coenzyme A reductase inhibitors, for example, statins (e.g., atorvastatin), together with one or more desiccants, which may be prepared using wet granulation methods.

Ingredients for tablets including, for example, triethylenetetramine dihydrochloride or triethylenetetramine disuccinate and atorvastatin, are provided in the below table:

| | Exemplary | Mg per | | Kg per |
|---|---|---|---|---|
| Ingredient | Function | tablet | % | 10 Kg |
| Triethylenetetramine | Copper | 300 | 67.26 | 6.726 |
| dihydrochloride or | binding | | | |
| triethylenetetramine | | | | |
| disuccinate | | | | |
| Atorvastatin | HMG-CoA | 10 | 2.24 | 0.224 |
| | reductase | | | |
| | inhibitor | | | |
| Lactose USP | Filler | 89.21 | 20.00 | 2.00 |
| Crosscarmellose | Disintegrant | 8.92 | 2.00 | 0.200 |
| sodium | | | | |
| Hydroxypropylcellulose | Granulation | 13.38 | 3.00 | 0.300 |
| | Binder | | | |
| Disodium phosphate | Desiccant | 22.30 | 5.00 | 0.500 |
| anhydrous | | | | |
| Magnesium stearate | Lubricant | 2.23 | 0.50 | 0.050 |
| Total | | 446.04 | 100 | 10 |

This tablet is prepared using wet granulation methods. The copper antagonist(s) is blended with the statin(s), and lactose in a suitable fluid bed granulator/dryer. Suitable granulator/dryers include Glatt or Niro fluid bed granulator/dryers. The hydroxypropylcellulose is dissolved in water or ethanol, and the blend is wet granulated with the solution of hydroxypropylcellulose in a suitable granulator/dryer. The wet granulation is dried in the granulator dryer. Where triethylenetetramine dihydrochloride is included in this formulation it is preferably precomplexed with a non-copper metal ion as disclosed herein, e.g., calcium, to enhance stability.

Alternatively this granulation can be prepared by blending the copper antagonist(s) with lactose and dicalcium phosphate and wet granulating with the hydroxypropylcellulose solution in a Niro or Glatt high speed granulator and drying it in, for example, a Glatt fluid bed dryer.

This granulation is mixed with the disodium phosphate and magnesium stearate or other lubricant in a suitable blender, such as Patterson Kelly V-blender. The final blend is compressed into tablets on a suitable tablet machine, such as a Manesty beta-press.

Copper antagonists other than triethylenetetramine dihydrochloride or triethylenetetramine disuccinate may be used, as may statins other than atorvastatin. Additionally, in certain cases, a copper antagonist compound which has already been considerably precomplexed with a non-copper metal ion as disclosed herein may be used, for example a triethylenetetramine precomplexed with calcium or another non-copper metal ion. Pentacoordinate copper antagonists may also be used, including for example, a triethylenetetramine complexed with calcium (or another non-copper metal) and another complexing agent, such as, for example, chloride, as disclosed herein.

Amounts of the copper antagonist(s) and statin(s), including the amounts of triethylenetetramine dihydrochloride or triethylenetetramine disuccinate and atorvastatin set forth in this Example, may be varied, as appropriate. By way of example only, the amount of triethylenetetramine dihydrochloride or triethylenetetramine disuccinate (or other copper antagonist) may range from about 1 mg to about 750 mg (for example, 1 mg, 5 mg, 10 mg, 25 mg, 50 mg, 100 mg, 125 mg, 150 mg 200 mg, 250 mg, 400 mg, 500 mg, 600 mg, and 750 mg), and the amount of atorvastatin may range from about 1 mg to about 40 mg (for example, 1 mg, 2 mg, 2.5 mg, 3 mg, 4 mg, 5 mg, 10 mg, 20 mg, 25 mg, 30 mg, 40 mg, and 80 mg). Other amounts may also be used. The amounts are not inflexible and may be determined, in part, for example, based on the number of tablets to be taken per day.

The tablet may be prepared for administration of drug, by way of example, in one or more doses, for example, one or two or more tablets once, twice, or more per day. Tablets are generally prepared for administration no more than four times per day, preferably less, and most preferably one or two times per day.

### EXAMPLE 18

### A Combination Capsule Employing Direct Filling

This Example describes preparation of capsules having a copper antagonist(s) such as, for example, one or more copper chelators (e.g., a trientine, such as triethylenetetramine dihydrochloride or triethylenetetramine disuccinate), and one or more 3-hydroxy-3-methylglutaryl coenzyme A reductase inhibitors, for example, statins (e.g., atorvastatin), which may be prepared using compaction and direct compression methods.

Ingredients for capsules including, for example, triethylenetetramine dihydrochloride or triethylenetetramine disuccinate and atorvastatin, are provided in the below table:

| | Exemplary | Mg per |
|---|---|---|
| Ingredient | Function | capsule |
| Triethylenetetramine | Copper | 100-300 |
| dihydrochloride or | binding | |
| triethylenetetramine | | |
| disuccinate | | |
| Atorvastatin | HMG-CoA | 2-10 |
| | reductase | |
| | inhibitor | |
| Silicon dioxide | Glidant; | 1.56 |
| | desiccant | |
| Magnesium stearate | Lubricant | 1.56 |

The process employs compaction. The copper antagonist(s) is/are first compacted, for example, in a suitable roller compacter. Suitable roller compactors include, for example, the Vector Mini-Model TF (Vector Corp., Marion, Iowa). It is then milled with a Fitz mill (Fitzpatrick Company, Elmhurst, Ill.) or other suitable mill, such as a Quadro Comill, oscillating mill, or pin mill, for example.

The milled copper antagonist is blended with the statin, silicon dioxide and magnesium stearate or other lubricant in a suitable blender. Suitable blenders include, for example, v-Blenders (Patterson-Kelly), planetary blenders (Hobart Corp., Troy Ohio).

The final blend is filled into hard gelatin capsules with on a suitable encapsulation machine, such as a Zanasi 40 E capsule machine.

Copper antagonists other than triethylenetetramine dihydrochloride or triethylenetetramine disuccinate may be used, as may statins other than atorvastatin. Additionally, in certain cases, a copper antagonist compound which has already been considerably precomplexed with a non-copper metal ion as disclosed herein may be used, for example a triethylenetetramine precomplexed with calcium or another non-copper metal ion. Pentacoordinate copper antagonists may also be used, including for example, a triethylenetetramine complexed with calcium (or another non-copper metal) and another complexing agent, such as, for example, chloride, as disclosed herein.

Amounts of the copper antagonist(s) and statin(s), including the amounts of triethylenetetramine dihydrochloride or triethylenetetramine disuccinate and atorvastatin set forth in this Example, may be varied, as appropriate. By way of example only, the amount of triethylenetetramine dihydrochloride or triethylenetetramine disuccinate (or other copper antagonist) may range from about 1 mg to about 750 mg (for example, 1 mg, 5 mg, 10 mg, 25 mg, 50 mg, 100 mg, 125 mg, 150 mg 200 mg, 250 mg, 400 mg, 500 mg, 600 mg, and 750 mg), and the amount of atorvastatin may range from about 1 mg to about 40 mg (for example, 1 mg, 2 mg, 2.5 mg, 3 mg, 4 mg, 5 mg, 10 mg, 20 mg, 25 mg, 30 mg, 40 mg, and 80 mg). Other amounts may also be used. The amounts are not inflexible and may be determined, in part, for example, based on the number of capsules to be taken per day.

A preferred capsule size is 300 mg. Thus, capsules may be prepared using appropriate doses within the ranges provided in order to yield a 300 mg size. The capsule may be prepared for administration of drug, by way of example, in one or more doses, for example, one or two or more tablets once, twice, or more per day. Capsules are generally prepared for administration no more than four times per day, preferably less, and most preferably one or two times per day.

### EXAMPLE 19

### A Combination Capsule Employing a Desiccant and Direct Filling

This Example describes preparation of capsules having a copper antagonist(s) such as, for example, one or more copper chelators (e.g., a trientine, such as triethylenetetramine dihydrochloride or triethylenetetramine disuccinate) and one or more 3-hydroxy-3-methylglutaryl coenzyme A reductase inhibitors, for example, statins (e.g., atorvastatin), together with one or more desiccants, which may be prepared using compaction and direct compression methods.

Ingredients for capsules including, for example, triethylenetetramine dihydrochloride or triethylenetetramine disuccinate and atorvastatin, are provided in the below table:

| | Exemplary | Mg per |
|---|---|---|
| Ingredient | Function | capsule |
| Triethylenetetramine | Copper | 100-300 |
| dihydrochloride or | binding | |
| triethylenetetramine | | |
| disuccinate | | |
| Atorvastatin | HMG-CoA | 2-10 |
| | reductase | |
| | inhibitor | |
| Disodium phosphate | Desiccant | 16.49 |
| anhydrous | | |
| Silicon dioxide | Glidant; | 1.65 |
| | desiccant | |
| Magnesium stearate | Lubricant | 1.65 |

The process employs compaction, blending and encapsulation. The copper antagonist(s) is/are first compacted, for example, in a suitable roller compacter. Suitable roller compactors include, for example, the Vector Mini-Model TF (Vector Corp., Marion, Iowa). It is then milled with a Fitz mill (Fitzpatrick Company, Elmhurst, Ill.) or other suitable mill, such as a Quadro Comill, oscillating mill, or pin mill, for example.

The milled copper antagonist is blended with the statin, disodium phosphate, silicon dioxide and magnesium stearate or other lubricant in a suitable blender. Suitable blenders include, for example, V-blenders (Patterson-Kelly), planetary blenders (Hobart Corp., Troy Ohio).

The final blend is encapsulated into hard gelatin capsules on a suitable capsule machine, such as a Zanasi 40E capsule machine.

Copper antagonists other than triethylenetetramine dihydrochloride or triethylenetetramine disuccinate may be used, as may statins other than atorvastatin. Additionally, in certain cases, a copper antagonist compound which has already been considerably precomplexed with a non-copper metal ion as disclosed herein may be used, for example a triethylenetetramine precomplexed with calcium or another non-copper metal ion. Pentacoordinate copper antagonists may also be used, including for example, a triethylenetetramine complexed with calcium (or another non-copper metal) and another complexing agent, such as, for example, chloride, as disclosed herein.

Amounts of the copper antagonist(s) and statin(s), including the amounts of triethylenetetramine dihydrochloride or triethylenetetramine disuccinate and atorvastatin set forth in this Example, may be varied, as appropriate. By way of example only, the amount of triethylenetetramine dihydrochloride or triethylenetetramine disuccinate (or other copper antagonist) may range from about 1 mg to about 750 mg (for example, 1 mg, 5 mg, 10 mg, 25 mg, 50 mg, 100 mg, 125 mg, 150 mg 200 mg, 250 mg, 400 mg, 500 mg, 600 mg, and 750 mg), and the amount of atorvastatin may range from about 1 mg to about 40 mg (for example, 1 mg, 2 mg, 2.5 mg, 3 mg, 4 mg, 5 mg, 10 mg, 20 mg, 25 mg, 30 mg, 40 mg, and 80 mg). Other amounts may also be used. The amounts are not inflexible and may be determined, in part, for example, based on the number of capsules to be taken per day.

A preferred capsule size is 300 mg. Thus, capsules may be prepared using appropriate doses within the ranges provided in order to yield a 300 mg size. The capsule may be prepared for administration of drug, by way of example, in one or more doses, for example, one or two or more tablets once, twice, or more per day. Capsules are generally prepared for administration no more than four times per day, preferably less, and most preferably one or two times per day.

### EXAMPLE 20

### A Combination Capsule Employing Fillers and Direct Filling

This Example describes preparation of capsules having a copper antagonist(s) such as, for example, one or more copper chelators (e.g., a trientine, such as triethylenetetramine dihydrochloride or triethylenetetramine disuccinate) and one or more 3-hydroxy-3-methylglutaryl coenzyme A reductase inhibitors, for example, statins (e.g., atorvastatin), together with one or more fillers, which may be prepared using direct filling methods.

Ingredients for capsules including, for example, triethylenetetramine dihydrochloride or triethylenetetramine disuccinate and atorvastatin, are provided in the below table:

| | Exemplary | Mg per |
|---|---|---|
| Ingredient | Function | capsule |
| Triethylenetetramine | Copper | 100-300 |
| dihydrochloride or | binding | |
| triethylenetetramine | | |
| disuccinate | | |
| Atorvastatin | HMG-CoA | 2-10 |
| | reductase | |
| | inhibitor | |
| Lactose USP | Filler | 80.00 |
| Crosscarmellose | Disintegrant | 8.00 |
| sodium | | |
| Magnesium stearate | Lubricant | 2.00 |

The process employs a combination of blending and direct encapsulation techniques. The copper antagonist is blended, for example, with the statin and lactose in a suitable blender. Suitable blenders include, for example, v-blenders (Patterson-Kelly), and planetary blenders (Hobart Corp).

The resulting blend is mixed with the crosscarmellose sodium in the same blender. This blend may be milled and screened in a Fitz mill (Fitzpatrick Corp) or other suitable mill, such as a Quadro Comill, oscillating mill, or pin mill, for example.

The resulting blend is mixed with the magnesium stearate, or other lubricant, which may also be accomplished in a suitable blender.

The final blend is filled into hard gelatin capsules on a suitable encapsulation machine, such as a Zanasi capsule machine.

Copper antagonists other than triethylenetetramine dihydrochloride or triethylenetetramine disuccinate may be used, as may statins other than atorvastatin. Additionally, in certain cases, a copper antagonist compound which has already been considerably precomplexed with a non-copper metal ion as disclosed herein may be used, for example a triethylenetetramine precomplexed with calcium or another non-copper metal ion. Pentacoordinate copper antagonists may also be used, including for example, a triethylenetetramine complexed with calcium (or another non-copper metal) and another complexing agent, such as, for example, chloride, as disclosed herein. Amounts of the copper antagonist(s) and statin(s), including the amounts of triethylenetetramine dihydrochloride or triethylenetetramine disuccinate and atorvastatin set forth in this Example, may be varied, as appropriate. By way of example only, the amount of triethylenetetramine dihydrochloride or triethylenetetramine disuccinate (or other copper antagonist) may range from about 1 mg to about 750 mg (for example, 1 mg, 5 mg, 10 mg, 25 mg, 50 mg, 100 mg, 125 mg, 150 mg 200 mg, 250 mg, 400 mg, 500 mg, 600 mg, and 750 mg), and the amount of atorvastatin may range from about 1 mg to about 40 mg (for example, 1 mg, 2 mg, 2.5 mg, 3 mg, 4 mg, 5 mg, 10 mg, 20 mg, 25 mg, 30 mg, 40 mg, and 80 mg). Other amounts may also be used. The amounts are not inflexible and may be determined, in part, for example, based on the number of capsules to be taken per day.

A preferred capsule size is 300 mg. Thus, capsules may be prepared using appropriate doses within the ranges provided in order to yield a 300 mg size. The capsule may be prepared for administration of drug, by way of example, in one or more doses, for example, one or two or more tablets once, twice, or more per day. Capsules are generally prepared for administration no more than four times per day, preferably less, and most preferably one or two times per day.

### EXAMPLE 21

### A Combination Capsule Employing Wet Granulation

This Example describes preparation of capsules having a copper antagonist(s) such as, for example, one or more copper chelators (e.g., a trientine, such as triethylenetetramine dihydrochloride or triethylenetetramine disuccinate) and one or more 3-hydroxy-3-methylglutaryl coenzyme A reductase inhibitors, for example, statins (e.g., atorvastatin), which may be prepared using wet granulation methods.

Ingredients for capsules including, for example, triethylenetetramine dihydrochloride or triethylenetetramine disuccinate and atorvastatin, are provided in the below table:

| | Exemplary | Mg per |
|---|---|---|
| Ingredient | Function | capsule |
| Triethylenetetramine | Copper | 100-300 |
| dihydrochloride or | binding | |
| triethylenetetramine | | |
| disuccinate | | |
| Atorvastatin | HMG-CoA | 2-10 |
| | reductase | |
| | inhibitor | |
| Lactose USP | Filler | 36.69 |
| Sodium starch glycollate | Disintegrant | 7.34 |
| Magnesium stearate | Lubricant | 1.83 |
| Hydroxypropylcellulose | Granulation | 11.00 |
| | binder | |

This tablet is prepared using wet granulation methods. The copper antagonist(s) is blended with the statin(s), and lactose and in a suitable fluid bed granulator/dryer. Suitable granulator/dryers include Glatt or Niro fluid bed granulator/dryers. The hydroxypropylcellulose is dissolved in water or ethanol, and the blend is wet granulated with the solution of hydroxypropylcellulose in a suitable granulator/dryer. The wet granulation is dried in the granulator dryer. Where triethylenetetramine dihydrochloride is included in this formulation it is preferably precomplexed with a non-copper metal ion as disclosed herein, e.g., calcium, to enhance stability.

Alternatively this granulation can be prepared by blending the copper antagonist(s) with lactose and wet granulating with the hydroxypropylcellulose solution in a Niro or Glatt high speed granulator and drying it in a Glatt fluid bed dryer.

This granulation is mixed with the sodium starch glycolate and magnesium stearate in a suitable blender, such as Patterson Kelly V-blender. The final blend is compressed into tablets on a suitable tablet machine, such as a Manesty beta-press.

Copper antagonists other than triethylenetetramine dihydrochloride or triethylenetetramine disuccinate may be used, as may statins other than atorvastatin. Additionally, in certain cases, a copper antagonist compound which has already been considerably precomplexed with a non-copper metal ion as disclosed herein may be used, for example a triethylenetetramine precomplexed with calcium or another non-copper metal ion. Pentacoordinate copper antagonists may also be used, including for example, a triethylenetetramine complexed with calcium (or another non-copper metal) and another complexing agent, such as, for example, chloride, as disclosed herein. Amounts of the copper antagonist(s) and statin(s), including the amounts of triethylenetetramine dihydrochloride or triethylenetetramine disuccinate and atorvastatin set forth in this Example, may be varied, as appropriate. By way of example only, the amount of triethylenetetramine dihydrochloride or triethylenetetramine disuccinate (or other copper antagonist) may range from about 1 mg to about 750 mg (for example, 1 mg, 5 mg, 10 mg, 25 mg, 50 mg, 100 mg, 125 mg, 150 mg 200 mg, 250 mg, 400 mg, 500 mg, 600 mg, and 750 mg), and the amount of atorvastatin may range from about 1 mg to about 40 mg (for example, 1 mg, 2 mg, 2.5 mg, 3 mg, 4 mg, 5 mg, 10 mg, 20 mg, 25 mg, 30 mg, 40 mg, and 80 mg). Other amounts may also be used. The amounts are not inflexible and may be determined, in part, for example, based on the number of capsules to be taken per day.

A preferred capsule size is 300 mg. Thus, capsules may be prepared using appropriate doses within the ranges provided in order to yield a 300 mg size. The capsule may be prepared for administration of drug, by way of example, in one or more doses, for example, one or two or more tablets once, twice, or more per day. Capsules are generally prepared for administration no more than four times per day, preferably less, and most preferably one or two times per day.

### EXAMPLE 22

### Combination Capsule Employing a Desiccant and Wet Granulation

This Example describes preparation of capsules having a copper antagonist(s) such as, for example, one or more copper chelators (e.g., a trientine, such as triethylenetetramine dihydrochloride or triethylenetetramine disuccinate) and one or more 3-hydroxy-3-methylglutaryl coenzyme A reductase inhibitors, for example, statins (e.g., atorvastatin), together with one or more desiccants, which may be prepared using wet granulation methods.

Ingredients for capsules including, for example, triethylenetetramine dihydrochloride or triethylenetetramine disuccinate and atorvastatin, are provided in the below table:

| | Exemplary | Mg per |
|---|---|---|
| Ingredient | Function | capsule |
| Triethylenetetramine | Copper | 300 |
| dihydrochloride or | binding | |
| triethylenetetramine | | |
| disuccinate | | |
| Atorvastatin | HMG-CoA | 10 |
| | reductase | |
| | inhibitor | |
| Lactose | Filler | 87.94 |
| Disodium phosphate | Desiccant | 21.99 |
| anhydrous | | |
| Crosscarmellose | Disintegrant | 4.40 |
| sodium | | |
| Magnesium stearate | Lubricant | 2.20 |
| Hydroxypropylcellulose | Granulation | 13.19 |
| | binder | |

This tablet is prepared using wet granulation methods. The copper antagonist(s) is blended with the statin(s) and lactose in a suitable fluid bed granulator/dryer. Suitable granulator/dryers include Glatt or Niro fluid bed granulator/dryers. The hydroxypropylcellulose is dissolved in water or ethanol, and the blend is wet granulated with the solution of hydroxypropylcellulose in a suitable granulator/dryer. The wet granulation is dried in the granulator dryer. Where triethylenetetramine dihydrochloride is included in this formulation it is preferably precomplexed with a non-copper metal ion as disclosed herein, e.g., calcium, to enhance stability.

Alternatively this granulation can be prepared by blending the copper antagonist(s) with lactose and dicalcium phosphate and wet granulating with the hydroxypropylcellulose solution in a Niro or Glatt high speed granulator and drying it in a Glatt fluid bed dryer.

This granulation is mixed with the disodium phosphate, crosscarmellose sodium and magnesium stearate in a suitable blender, such as Patterson Kelly V-blender. The final blend is filled into hard gelatin capsules in a suitable encapsulation machine, such as a Zanasi 40E capsule machine.

Copper antagonists other than triethylenetetramine dihydrochloride or triethylenetetramine disuccinate may be used, as may statins other than atorvastatin. Additionally, in certain cases, a copper antagonist compound which has already been considerably precomplexed with a non-copper metal ion as disclosed herein may be used, for example a triethylenetetramine precomplexed with calcium or another non-copper metal ion. Pentacoordinate copper antagonists may also be used, including for example, a triethylenetetramine complexed with calcium (or another non-copper metal) and another complexing agent, such as, for example, chloride, as disclosed herein. Amounts of the copper antagonist(s) and statin(s), including the amounts of triethylenetetramine dihydrochloride or triethylenetetramine disuccinate and atorvastatin set forth in this Example, may be varied, as appropriate. By way of example only, the amount of triethylenetetramine dihydrochloride or triethylenetetramine disuccinate (or. other copper antagonist) may range from about 1 mg to about 750 mg (for example, 1 mg, 5 mg, 10 mg, 25 mg, 50 mg, 100 mg, 125 mg, 150 mg 200 mg, 250 mg, 400 mg, 500 mg, 600 mg, and 750 mg), and the amount of atorvastatin may range from about 1 mg to about 40 mg (for example, 1 mg, 2 mg, 2.5 mg, 3 mg, 4 mg, 5 mg, 10 mg, 20 mg, 25 mg, 30 mg, 40 mg, and 80 mg). Other amounts may also be used. The amounts are not inflexible and may be determined, in part, for example, based on the number of capsules to be taken per day.

A preferred capsule size is 300 mg. Thus, capsules may be prepared using appropriate doses within the ranges provided in order to yield a 300 mg size. The capsule may be prepared for administration of drug, by way of example, in one or more doses, for example, one or two or more tablets once, twice, or more per day. Capsules are generally prepared for administration no more than four times per day, preferably less, and most preferably one or two times per day.

### EXAMPLE 23

### A Controlled Release Combination Tablet

This Example describes preparation of matrix controlled release tablets having a copper antagonist(s) such as, for example, one or more copper chelators (e.g., a trientine, such as triethylenetetramine dihydrochloride or triethylenetetramine disuccinate), and one or more 3-hydroxy-3-methylglutaryl coenzyme A reductase inhibitors, for example, statins (e.g., atorvastatin), which may be prepared using roller compaction and direct compression methods.

Ingredients for tablets including, for example, triethylenetetramine dihydrochloride or triethylenetetramine disuccinate and atorvastatin, are provided in the below table:

| | Exemplary | Mg per | | | Kg per |
|---|---|---|---|---|---|
| Ingredient | Function | tablet | | % | 10 Kg |
| Triethylenetetramine | Copper | 300 | mg | 67.26 | 6.726 |
| dihydrochloride or | binding | | | | |
| triethylenetetramine | | | | | |
| disuccinate | | | | | |
| Atorvastatin | HMG-CoA | 10 | | 2.24 | 0.224 |
| | reductase | | | | |
| | inhibitor | | | | |
| Hydroxypropylmethyl- | Controlled | 89.21 | | 20.00 | 2.00 |
| cellulose | release agent | | | | |
| Lactose | Filler | 44.60 | | 10.00 | 1.00 |
| Magnesium stearate | Lubricant | 2.23 | | 0.50 | 0.050 |
| Total | | 446.04 | | 100 | 10 |

The process employs compaction and direct compression. The copper antagonist(s) is/are first compacted, for example, in a suitable roller compacter. Suitable roller compactors include, for example, the Vector Mini-Model TF (Vector Corp., Marion, Iowa). It is then milled with a Fitz mill (Fitzpatrick Company, Elmhurst, Ill.) or other suitable mill, such as a Comill mill, oscillator mill, or pin mill, for example.

The milled copper antagonist is blended with statin, hydroxypropyl-methylcellulose, and lactose in a suitable blender. Suitable blenders include V-Blenders (Patterson-Kelly, and planetary blenders (Hobart Corp., Troy Ohio).

This blend is blended with the magnesium stearate or other lubricant in the same blender.

The final blend is compressed into tablets using a suitable tablet machine, such as a Manesty beta-press (Manesty, Knowsley, Merseyside, UK).

Copper antagonists other than triethylenetetramine dihydrochloride or triethylenetetramine disuccinate may be used, as may statins other than atorvastatin. Additionally, in certain cases, a copper antagonist compound which has already been considerably precomplexed with a non-copper metal ion as disclosed herein may be used, for exanple a triethylenetetramine precomplexed with calcium or another non-copper metal ion. Pentacoordinate copper antagonists may also be used, including for example, a triethylenetetramine complexed with calcium (or another non-copper metal) and another complexing agent, such as, for example, chloride, as disclosed herein. Amounts of the copper antagonist(s) and statin(s), including the amounts of triethylenetetramine dihydrochloride or triethylenetetramine disuccinate and atorvastatin set forth in this Example, may be varied, as appropriate. By way of example only, the amount of triethylenetetramine dihydrochloride or triethylenetetramine disuccinate (or other copper antagonist) may range from about 1 mg to about 750 mg (for example, 1 mg, 5 mg, 10 mg, 25 mg, 50 mg, 100 mg, 125 mg, 150 mg 200 mg, 250 mg, 400 mg, 500 mg, 600 mg, and 750 mg), and the amount of atorvastatin may range from about 1 mg to about 40 mg (for example, 1 mg, 2 mg, 2.5 mg, 3 mg, 4 mg, 5 mg, 10 mg, 20 mg, 25 mg, 30 mg, 40 mg, and 80 mg). Other amounts may also be used. The amounts are not inflexible and may be determined, in part, for example, based on the number of tablets to be taken per day.

The tablet may be prepared for administration of drug, by way of example, in one or more doses, for example, one or two or more tablets once, twice, or more per day. Tablets are generally prepared for administration no more than four times per day, preferably less, and most preferably one or two times per day.

### EXAMPLE 24

### A Combination Capsule Containing Enteric Coated Beads

This Example describes preparation of capsules containing enteric beads-having a copper antagonist(s) such as, for example, one or more copper chelators (e.g., a trientine, such as triethylenetetramine dihydrochloride or triethylenetetramine disuccinate), and one or more 3-hydroxy-3-methylglutaryl coenzyme A reductase inhibitors, for example, statins (e.g., atorvastatin), which may be prepared using granulation, spheronization, and bead coating methods.

Ingredients for capsules including, for example, triethylenetetramine dihydrochloride or triethylenetetramine disuccinate and atorvastatin, are provided in the below table:

| | Exemplary | Mg per |
|---|---|---|
| Ingredient | Function | capsule |
| Triethylenetetramine | Copper | 300 |
| dihydrochloride or | binding | |
| triethylenetetramine | | |
| disuccinate | | |
| Atorvastatin | HMG-CoA | 10 |
| | reductase | |
| | inhibitor | |
| Hydroxypropylcellulose | Granulation | 10.70 |
| | binder | |
| Cellulose acetate | Delayed | 35.67 |
| phthalate | release agent | |
| Talc | Anti adherent | 0.36 |

The copper antagonist(s), statin(s), and hydroxypropylcellulose are blended in a suitable granulator-spheronizer, such as a Niro Roto-Processor spheronizer. Water or alcohol is used to wet the granulation and the wet mass is spheronized to beads on the processor. The beads are dried in a fluid bed coating/drying processor, such as a Niro Precision coater.

A commercial aqueous or alcohol solution of cellulose acetate phthalate, for example, Aquacote CPD-FMC Corporation, is used to coat the beads in the coating-drying processor. The dried beads are coated with the solution and dried in fluid bed coating apparatus. Talc can be added to keep the beads free flowing. The beads are filled into hard gelatin capsules using an appropriate capsule-filling machine, such as a Zanasi encapsulation machine

Copper antagonists other than triethylenetetranine dihydrochloride or triethylenetetramine disuccinate may be used, as may statins other than atorvastatin. Additionally, in certain cases, a copper antagonist compound which has already been considerably precomplexed with a non-copper metal ion as disclosed herein may be used, for example a triethylenetetramine precomplexed with calcium or another non-copper metal ion. Pentacoordinate copper antagonists may also be used, including for example, a triethylenetetramine complexed with calcium (or another non-copper metal) and another complexing agent, such as, for example, chloride, as disclosed herein. Amounts of the copper antagonist(s) and statin(s), including the amounts of triethylenetetramine dihydrochloride or triethylenetetramine disuccinate and atorvastatin set forth in this Example, may be varied, as appropriate. By way of example only, the amount of triethylenetetramine dihydrochloride or triethylenetetramine disuccinate (or other copper antagonist) may range from about 1 mg to about 750 mg (for example, 1 mg, 5 mg, 10 mg, 25 mg, 50 mg, 100 mg, 125 mg, 150 mg 200 mg, 250 mg, 400 mg, 500 mg, 600 mg, and 750 mg), and the amount of atorvastatin may range from about 1 mg to about 40 mg (for example, 1 mg, 2 mg, 2.5 mg, 3 mg, 4 mg, 5 mg, 10 mg, 20 mg, 25 mg, 30 mg, 40 mg, and 80 mg). Other amounts may also be used. The amounts are not inflexible and may be determined, in part, for example, based on the number of capsules to be taken per day.

A preferred capsule size is 300 mg. Thus, capsules may be prepared using appropriate doses within the ranges provided in order to yield a 300 mg size. The capsules may be prepared for administration of drug, by way of example, in one or more doses, for example, one or two or more tablets once, twice, or more per day. Capsules are generally prepared for administration no more than four times per day, preferably less, and most preferably one or two times per day.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

All of the features disclosed in this specification may be combined in any combination. Thus, unless expressly stated otherwise, each feature disclosed is only an example of a generic series of equivalent or similar features.

It is to be understood that while the invention has been described in conjunction with the detailed description thereof, the foregoing description is intended to illustrate and not limit the scope of the invention, which is defined by the scope of the appended claims. Thus, from the foregoing, it will be appreciated that, although specific embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various embodiments and/or preferred embodiments and optional features, any and all modifications and variations of the concepts herein disclosed that may be resorted to by those skilled in the art are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, the term “X and/or Y” means “X” or “Y” or both “X” and “Y”, and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member. and any subgroup of members of the Markush group, and applicants reserve the right to revise the application or claims to refer specifically to any individual member or any subgroup of members of the Markush group.

Other embodiments are within the following claims. The patent may not be interpreted to be limited to the specific examples or embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

1. A composition comprising a pharmaceutically acceptable carrier and therapeutically effective amounts of a copper (II) chelator and a 3-hydroxy-3-methylglutaryl coenzyme A reductase inhibitor.

2. The composition of claim 1 wherein said 3-hydroxy-3-methylglutaryl coenzyme A reductase inhibitor is selected from the group consisting of simvastatin, atorvastatin, mevastatin, lovastatin, pravastatin, fluvastatin, rosuvastatin, itavastatin, and visastatin.

3. The composition of claim 1 wherein said copper (II) chelator is selected from the group consisting of 2,3,2 tetramine, 2,2,2 tetramine, and 3,3,3 tetramine.

4. The composition of claim 1 wherein said copper (II) chelator is a triethylenetetramine.

5. The composition of claim 1 wherein said copper (II) chelator is a triethylenetetramine salt.

6. The composition of claim 1 wherein said copper (II) chelator is a triethylenetetramine succinate salt.

7. The composition of claim 6 wherein said triethylenetetramine succinate salt is triethylenetetramine disuccinate.

8. A method of treating a subject for elevated copper and/or LDL-C, comprising administering to said subject a composition comprising a carrier and therapeutically effective amounts of a copper (II) chelator and a 3-hydroxy-3-methylglutaryl coenzyme A reductase inhibitor.

9. The method of claim 8 wherein said 3-hydroxy-3-methylglutaryl coenzyme A reductase inhibitor is selected from the group consisting of simvastatin, atorvastatin, mevastatin, lovastatin, pravastatin, fluvastatin, rosuvastatin, itavastatin, and visastatin.

10. The method of claim 8 wherein said copper (II) chelator is. selected from the group consisting of 2,3,2 tetramine, 2,2,2 tetramine, and 3,3,3 tetramine.

11. The method of claim 8 wherein said copper (II) chelator is a triethylenetetramine.

12. The method of claim 8 wherein said copper (II) chelator is a triethylenetetramine salt.

13. The method of claim 8 wherein said copper (II) chelator is a triethylenetetramine succinate salt.

14. The method of claim 13 wherein said triethylenetetramine succinate salt is triethylenetetramine disuccinate.

15. The method of claim 8 wherein said subject is a human with diabetes mellitus.

16. The method of claim 8 wherein said subject is a human with atherosclerosis.

17. The method of claim 8 wherein said subject is a human with coronary heart disease.

18. The method of claim 8 wherein said subject is a human with Syndrome X.

19. The method of claim 8 wherein said subject is a human that is hypertensive.

20. The method of claim 8 wherein said subject is a human that is obese.
